SEQUENCE LISTING (147 sequences)

SEQ ID NO: 1 - DNA, 24 bp, Artificial Sequence
  Degenerate oligonucleotide forward direction Alc1a2
tgygayccntgycgnaarggnaaa

SEQ ID NO: 2 - DNA, 24 bp, Artificial Sequence
  Degenerate oligonucleotide forward direction Alc1b2
tgygayccntgycgnaarggnaag

SEQ ID NO: 3 - DNA, 24 bp, Artificial Sequence
  Degenerate oligonucleotide forward direction Alc1c2
tgygayccntgycgraarggnaaa

SEQ ID NO: 4 - DNA, 24 bp, Artificial Sequence
  Degenerate oligonucleotide forward direction Alc1d2
tgygayccntgycgraarggnaag

SEQ ID NO: 5 - DNA, 21 bp, Artificial Sequence
  Degenerate oligonucleotide reverse complement Alcrev1a
cctncgyttrcarttngarca

SEQ ID NO: 6 - DNA, 21 bp, Artificial Sequence
  Degenerate oligonucleotide reverse complement Alcrev1b
cctncgyttrcarttrctrca

SEQ ID NO: 7 - DNA, 21 bp, Artificial Sequence
  Degenerate oligonucleotide reverse complement Alcrev1c
cctycgyttrcarttngarca

SEQ ID NO: 8 - DNA, 21 bp, Artificial Sequence
  Degenerate oligonucleotide reverse complement Alcrev1d
cctycgyttrcarttrctrca

SEQ ID NO: 9 - DNA, 214 bp, Aspergillus ustus
gaattcgccctttgtgayccgtgyagraaagggagrcgagggtgtgatgcgcctgtgagttgactcgtgcctacctgcctcgcttcaaaggcagaatcaggccatacgcgccctatgcctgcgaagaatccggaattctctaacgccactccaggaaaatcgaagtggagatggatacacctgctccaactgyaagmgvaggaagggcgaattc

SEQ ID NO: 10 - DNA, 156 bp, Aspergillus fumigatus
gaattcgccctttgtgatccgtgtcggaaggggaagcgggcgtgcgatgcgcctgctcgtagagaccggcacgcggacgccggcagccgaagggtgctagcagagagcaacctcaacatcccgtgctccaactgcaarcgcaggaagggcgaattc

SEQ ID NO: 11 - DNA, 162 bp, Aspergillus versicolor
  misc_feature (58)..(58): n represents c, g, a or t
gaattcgccctttgtgatccgtgtcggaaggggaagcgagggtgtgatgcgcctgttngttgacaccggcaaagatcttaaacgcgaatccgaaagtgccactcgagaaaatggcaactggatactcgtgctccaactgcaagcgcaggaagggcgaattcn

SEQ ID NO: 12 - DNA, 24 bp, Artificial Sequence
  Degenerate oligonucleotide reverse complement Alc7001a
athtaycaygaytcnatggaraat

SEQ ID NO: 13 - DNA, 24 bp, Artificial Sequence
  Degenerate oligonucleotide reverse complement Alc7001b
athtaycaygaytcnatggaraac

SEQ ID NO: 14 - DNA, 24 bp, Artificial Sequence
  Degenerate oligonucleotide reverse complement Alc7001c
athtaycaygayagyatggaraat

SEQ ID NO: 15 - DNA, 24 bp, Artificial Sequence
  Degenerate oligonucleotide reverse complement Alc7001d
athtaycaygayagyatggaraac

SEQ ID NO: 16 - DNA, 816 bp, Aspergillus flavus
gaattcgccctttgtgatccgtgycggaaagggaagagagcatgcgatgccctcctggctgacgagcttgaacggaattccaacactgctgctcgacaagcgtacaatcacgcgtgctccaactgcaaaaaatacaaaagaaaatgcacgttcgactggctcttgagtcacaaggaatcccggcatgctcatagcaagagagccagaaatatcgcgatcgccctctcgcggcaggtgaacgattgttccgctcattcctctcaacaaacctccactgggcgcaatcctacagagctccctctgcaaaacatcgaggattgcgaatggccaacgtctgttagggacccgcttttgccgttcccacaagacgaggaactagatgcggactggttaacttggggatgcctcaacgacgcagtgtccatctctcctctaagcgccgacatgactctcaatggggataggcacgtcaatcctaaccagacaccacaaatgagtactcaatggaactctgtcggggccggccaggcatggcaaagtatcggtcaaacttcactgctcgacacgatgaacagttctataacttcgtcgcaattcaaggatacacccgactatcgatcatttgagacatgggatatcagttctgggctcccgcttcacggtcttccacctaccgaaggacgtggtgtgtcgatgccaacaaacactacactgtgtgtgggctcaaaccaattagcacacaattatgcgcactccatgatgacgcgcaacctaattcacatmtaccacgacagcatggaaaataagggcgaattc

SEQ ID NO: 17 - DNA, 2627 bp, Aspergillus fumigatus
gaattcgcccttatggaggctcatcgtcgacgccagcaccacagctgcgatccatgtcggaaggggaagcgggcgtgcgatgcgcctgctcgtagagaccggcacgcggacgccggcagccgaagggtgctagcagagagcaacctcaacatcccgtgctcgaactgcaggaaatacaatcgagaatgcacgttcaactggttagtcgagaaccgcgccgccgcacgggcgggtcgaaagcagaagagccgtaatgtgagcaacttgcctcgagcggacgacgtgagttcgagtcgctcgggaaccgacctgctggacgatctgcggtactcctcgtcgtggctatccaacagtcctgggaatggggtgtcgtcgaacggttcgacggaggaccagcccgggacgtggtcgatgccgtcgaatgccgtctcgataccgctgagaagcaaggagtcggaactcgatccgttcagtgtcatgctgtggaatgcaaatacagcacacgtaccgccgagcaatgcggaaacggcgggctcggctgaggacacttgttcgagtctggactactaccagcagagcttgtccagttcgggaccgcactcgctcgacgagacgctagatctacttcaacagttcgatgattcgagtccaggattgagtagctcgtattactcttcgccacctggctttgtgattccggaaggtagtgacggtctaccgacattcccggcagacagtctctatccctccgggaacaaagatagtctatttgttctttccgataacatctcagacagctatgcccgctcgatgatgacacagaatcttatccgcatataccatgacagcatggagaatgcgttgtcctgctggctcacggagcaaaactgtccctacaacacggcagtcccgtacacctcaccgagcgggctcgccagtaaggcacaagcggcatgggccccgaactggacgaaccggatctgtactcgggtctgtcggctcgatcgagcgtatgcatccgtccgtgggcgaaacctcagcgccgcagaagagaaaatggcatcgagagcgctccacaccgccatcatggcgttcgcctcgcagtgggcgcagaagatgcccagaagcaatggcttttctcttacctcgcccgtcgcgcagcacgagcgtgtcatccgggagaatctgtggaaccaggcgcggcgtgctctggagaatgcagcgggtatcccttcgttccgggttgcgtttgcgaacatcatcttctccatcggacagcgtccgctcaatgtcgatgaggacatggagctgcatgagttgctggagaatgacagcgcgccgttgttcatggaggcggcggtgcgacagctgttttcaatccgatataaactgacccgtctcgagcggcagaagccaaagtcgcgaagttcgccagagcagagcaagatcgatctcgccagtatggatatgccgtcgccacagacggatgcgttctatgccgacccggagcaccaggaaaccgtcaacctcctgttctggctggtggtcatgttcgacaccctgcaggcggccatgtatcagcgtcccctcgccatctccgacgaggacagccagatcacgtccgtgtcaccggcggtctccaacgccaaacccgacagcagcgtcgacctcgacggctggaacatcacgtactcccgcgccctgaaagagaaacaagacctctggggcgacttcttcctccacaaacgcgccgcacgccagggcgcgaacccaccccgctggccctgctcctacgaagaagccgccgagatcctctccgacgccagccccgtcaaagtcctcctcttccgacaagtcacccgcctccagaccctcgtctaccgcggcgccagtcccgaccgcctggaggagatcatccaaaagacgctgcgcatctaccaacactggaacaccacctacaagcaattcttccagagctgcaacgcaaaccacgacgatctgcccccgcgcatccagtcgtggtacgtcatcgtcgcagggcactggcatctcgccgccatgctgctcgccgacaccgtcaagggcatcgacgagggccacctcggcctggacagccggcgcgaagcccgcaccgcaatcgacttcgtcgccaccctccggcgggacaacgcgctggccgtcggggccatcgctcagcgctccctgcaggggcgggactccctggccaaccgcatccagttctaccacgacgccgtgaacgaggccgcgtttctgacggagccgtggacgctcgtcctgattcgctgtttcgccaaggcggcgtatattctgctagacgacatcacgccgcagtcgcacggcgcgcggccggacgacccgtccgagtacgcccggcggaactgcgagttctgtatctcggcgctgtggtgtctggggacgaaatcggacatggcgtttgtggctgcgcgctcgttgtcgaagctgctggatacgcgactagggaaaggtgtcgatcagttctgttccgtaggggagggtgctcggattccgtccatgccgctttttgatgaacggggatcgggcgagttgggcagtgtcgggatctcggtgtagttaagggcgaattc

SEQ ID NO: 18 - protein, 866 aa, Aspergillus fumigatus
MEAHRRRQHHSCDPCRKGKRACDAPARRDRHADAGSRRVLAESNLNIPCSNCRKYNRECTFNWLVENRAAARAGRKQKSRNVSNLPRADDVSSSRSGTDLLDDLRYSSSWLSNSPGNGVSSNGSTEDQPGTWSMPSNAVSIPLRSKESELDPFSVMLWNANTAHVPPSNAETAGSAEDTCSSLDYYQQSLSSSGPHSLDETLDLLQQFDDSSPGLSSSYYSSPPGFVIPEGSDGLPTFPADSLYPSGNKDSLFVLSDNISDSYARSMMTQNLIRIYHDSMENALSCWLTEQNCPYNTAVPYTSPSGLASKAQAAWAPNWTNRICTRVCRLDRAYASVRGRNLSAAEEKMASRALHTAIMAFASQWAQKMPRSNGFSLTSPVAQHERVIRENLWNQARRALENAAGIPSFRVAFANIIFSIGQRPLNVDEDMELHELLENDSAPLFMEAAVRQLFSIRYKLTRLERQKPKSRSSPEQSKIDLASMDMPSPQTDAFYADPEHQETVNLLFWLVVMFDTLQAAMYQRPLAISDEDSQITSVSPAVSNAKPDSSVDLDGWNITYSRALKEKQDLWGDFFLHKRAARQGANPPRWPCSYEEAAEILSDASPVKVLLFRQVTRLQTLVYRGASPDRLEEIIQKTLRIYQHWNTTYKQFFQSCNANHDDLPPRIQSWYVIVAGHWHLAAMLLADTVKGIDEGHLGLDSRREARTAIDFVATLRRDNALAVGAIAQRSLQGRDSLANRIQFYHDAVNEAAFLTEPWTLVLIRCFAKAAYILLDDITPQSHGARPDDPSEYARRNCEFCISALWCLGTKSDMAFVAARSLSKLLDTRLGKGVDQFCSVGEGARIPSMPLFDERGSGELGSVGISV

SEQ ID NO: 19 - DNA, 26 bp, Artificial Sequence
  Oligonucleotide AF Alc gen1
tgcgatgcgcctgctcgtagagaccg

SEQ ID NO: 20 - DNA, 27 bp, Artificial Sequence
  Oligonucleotide AFAlcgen1
agggtgctagcagagagcaacctcaac

SEQ ID NO: 21 - DNA, 27 bp, Artificial Sequence
  Oligonucleotide Alcfum walk3a
cgtgctctggagaatgcagcgggtatc

SEQ ID NO: 22 - DNA, 27 bp, Artificial Sequence
  Oligonucleotide Alcfum walk3
gctgcatgagttgcaggagaatgacag

SEQ ID NO: 23 - DNA, 24 bp, Artificial Sequence
  Sense oligonucleotide (fum for)
atggaggctcatcgtcgacgccag

SEQ ID NO: 24 - DNA, 24 bp, Artificial Sequence
  Antisense oligonucleotide (Fum rev)
aactacaccgagatcccgacactg

SEQ ID NO: 25 - DNA, 997 bp, Aspergillus fumigatus
gaattcgcccttactatagggcacgcgtggtcgacggcccgggctggtatccttgctacactgctaaacaacggcacctcacccatcaccggcaagcgaatcctcgagacaaccacagtcgacgagatgttccgcaatcagatccccaacctccccaattttgccgcacaaggcatccctccttcgaagcctgacctcaccaatgaaatagctcatctgtacccatcgccgacacctcaggggtggggcctcacctttatgctgacgggagggtccactggacggtctgaagggacggcgcactgggcaggacttgcgaacctctggtggtggtgcgatagggagaaaggggtcgcagggatgatttgtactcaactcttgccctttgctgatccccaagtttggagcctttggctggatgtggagtctgccgtctaccgtggcctggctcaggattagactctgccgtatcaattgctcctcctgagatatttctatatgattggactagtttccatcagtcagtccgttcttttgttttttttttttttttttttatagactttgaactcaatacctccggtcatccgaagctggcrtgctgaakcgctsaakkggyrtwcmycaskrgrtrtgwcmsytygcmaamamraagyskwragmkcwwccgccaycgcagtccaaccacccaaccagcgcatcactcggacgcaaacagactcaacgactcgtcctagtgcgccgacaatccaggcagcgataaaccagtcaggtctcgtgaactccctcccagaaccaccagacttcgcgaatccccagaccccgcatcgtgctcttggctcggagcttcaaracccgcctagccatgaggtggtctctctcacactgtatcccccctccccccatatctctctccacaatagccatcacccggtaatagccgaatttgtatgccggcataccgtagcgcttggagacaactgtcagtgccacgatg

SEQ ID NO: 26 - DNA, 27 bp, Artificial Sequence
  Oligonucleotide  Alcfum walkup1
gttgaggttgctctctgctagcaccct

SEQ ID NO: 27 - DNA, 26 bp, Artificial Sequence
  Oligonucleotide Alcfum walkup2
cggtctctacgagcaggcgcatcgca

SEQ ID NO: 28 - DNA, 27 bp, Artificial Sequence
  Oligonucleotide Alcust walk1
cgcttcaaaggcagaatcaggccatac

SEQ ID NO: 29 - DNA, 27 bp, Artificial Sequence
  Oligonucleotide Alcust walk2
atccggaattctctaacgccactccag

SEQ ID NO: 30 - DNA, 27 bp, Artificial Sequence
  Oligonucleotide Alcust walk3
atgccgacccgatgagcgcaatgctac

SEQ ID NO: 31 - DNA, 27 bp, Artificial Sequence
  Oligonucleotide Alcust walk4
atacgcggaagggcactgagcgtagac

SEQ ID NO: 32 - DNA, 26 bp, Artificial Sequence
  Oligonucleotide Alcust walk5
ctacaacactccacagggatcccgtc

SEQ ID NO: 33 - DNA, 27 bp, Artificial Sequence
  Oligonucleotide Alcust walk6
cacagagtccgctggacgagaatcgac

SEQ ID NO: 34 - DNA, 27 bp, Artificial Sequence
  Oligonucleotide Alcust walkup1
ctggagtggcgttagagaattccggat

SEQ ID NO: 35 - DNA, 27 bp, Artificial Sequence
  Oligonucleotide Alcust walkup2
ctatggcctgattctgcctttgaagcg

SEQ ID NO: 36 - DNA, 22 bp, Artificial Sequence
  Sense oligonucleotide (Alcust for)
ctcgaatgaagatgggagactc

SEQ ID NO: 37 - DNA, 22 bp, Artificial Sequence
  Antisense oligonucleotide (Alcust rev)
ttacacaaggatatccgctgac

SEQ ID NO: 38 - DNA, 2592 bp, Aspergillus ustus
gaattcgcccttctcgaatgaagatgggagactcccgtcgccgccagaatcatagctgcgatccgtgtcgcaaggggaaacgagggtgtgatgcgcctgtgagttgactcgtgcctacctgcctcgcttcaaaggcagaatcaggccatacgcgccctatgcctgcgaagaatccggaattctctaacgccactccaggaaaatcgaagtggagatggatacacctgctcgaattgcaagcggtggaagaagaaatgcacattcaatttcgtctcgtccaggcgcgcagattcccgcgtcgtcggtgccaatgcccggtcaaaagcgaagtccacctctacccctgctgtctctaccgctgcatcggtagccacttctgcagctgcccctcccactcccgatagtggcgacatccctgccatgctaaacacgggtatggacatgggcacgaatgagtacgatgctctccttcatgacggtttgcggtcgtcacaccttgaccctacgaggcttggggatatgtttgcttttacctcgccgtctagtttcacggcggaggctttgcatgcgcagagtgctgttggcacagaagccatcgcgtgggattcagggattccaacagactggtctatcccttcgatgcctcggtcggaaaagtcgttcactccgcttgagagtcaggcggtctttcttgcacaggaggattcgaaccagtttgacgttattcaggagttggaagatggctcatccgacaacttcacaccaccggggcggaaacgcgacgaggataagcgacggaaatttcaatgggagttatgcatcgcttccgacaaaacagccaaccaggttggccgatcgacaatgacgcgcaatctaatgcggatatatcacgatagcatggagaatgcgctctcatgttggttgaccgagcacaactgtccgtatgccgacccgatgagcgcaatgctaccttttaaccagaggaaagaatggggtcccagttggtcgaacaggatgtgtatccgggtctgtcatttagatcgggaatcatcctcgatacgcggaagggcactgagcgtagacgaggaccggacggccgcgcgggcgctgcatctcgcaattgtcgcattcgcctcacagtggacgcagcatgcccaaagggggacagggctttcggttccgactgatatcgctacgatgaacggtcgattcgaaagaatatatggaacgaggcgcggcatgctctacaacactccacagggatcccgtctttccgggtaatattcgccaacattattttctcattgacacagagtccgctggacgagaatcgacctgcgaagctaggtcagctgttggagaatgatggtgctcccgtattcctagagaacgccaatcgtcagctctacacattccgacacaagttcgcgagactccaacgagaggctcccccgcctgtggctgggctgcgacgaggttcaatatcatccactctcactgacgtgctggaagttccgactcctgaatctccacaggtcgatccaattctcgcgaatcaagaccaccgaagcacactcagcctcctcttctggcttggaatcatgttcgacaccctcagtgcagccatgtaccagcgccctcttgtcgtctcagacgaagatagccaaatcgcctccgcctccccgtcggcctcaaccaacccccgagtcaacctcaactattgggaaatcccagacagcaatctcccagcgaaaaacgacgtctggggtgaatttttccttcaacctgccgctcgccaggaactggcctccgcacatccccaaatccaaccaaaacaaccccgttggccgtgttcctacgaagaagccgcatcagtcctgtccgaggcaacaccggtaaaagtccttctctaccgccgsgtcacccaactccaaacccttatctaccgtggcgcgtctcccgcacggcttgaagaagtcattcaaagaacgcttctcgtctaccaccattggacctgcacatatcaatcatttatgctcgactgtgtggcaaaccacgagtcccttccacaccgtattcagtcttggtatgttattcttgatggccattggcacctctccgcaatgcttctcgccgatgtgctagagtccatcgacagaagccacctcggactcgagtcggagcgcgagtcccggattgcaagcgatcttattgcaacactgcgaatcgacaatgcactcgcagtcggtgccttggctagggcatcgctacacggggagaatagcatgatgcatcgacatttccatgactcgttgaacgaggtcgcgttcctggttgagccgtggacagtcgttttggtccattgtttcgcgaaggcggcggctatttcgctggattgtctgggtcagggacagggaggtgctttggcagaatgttttcggcagaattgtgaatattgtatttgtgcgttgaagtatttgggacggaaatcggacatggcgttttgtgttgcgggcgggttggagaaggagttgcttgagaaagctgggagtatgctgtcagcggatatccttgtgtaaaagggcgaattcc

SEQ ID NO: 39 - DNA, 619 bp, Aspergillis ustus
  misc_feature (617)..(617): Tranlation start site
tcgagaatatacgaagtcaagactgtcngtgtacagctcaaggcttaagcagaatgttctragaatatggtytggtagttacatgttcctagtatgctttgatgatctattagtctcgtatacarggaagacagtatgatgttagtatgtataagaagagactagctacggtgatgttaagaacttacgttcaagatgccgtataatttccgaatactccagagtataactccggatcgccacctcgtagctcttaaataagcaattccaattctgcgagtgcgacgtatcaaccaagtgtcggactgcgggggcgatctccgccccgagagttcacgctaggcccagcactgcatcgcccccacagcgaggtatrgkccycgcctgctattggcctcgtgccccgcgcacatcctcaccggagtcggaggcagcaggaacttggggctggtcatgtgacagcaaaccccgcagagcccaatggttgactttccccagaatctcgyccagctgcgacaaatcccgccttccccaactcccgtctcggagattgtctccacgtccttgttagaataatcatcaattccgaattgatacgttacgtatcgtacctcgaatg

SEQ ID NO: 40 - DNA, 27 bp, Artificial Sequence
  Oligonucleotide Alcvers walk1
aggcgctgtgatgctccggtttgtggc

SEQ ID NO: 41 - DNA, 28 bp, Artificial Sequence
  Oligonucleotide Alcvers walk2
tgatatcaaatacttcttagagcaaccg

SEQ ID NO: 42 - DNA, 28 bp, Artificial Sequence
  Oligonucleotide Alcvers walkup1
cggttgctctaagaagtatttgatatca

SEQ ID NO: 43 - DNA, 27 bp, Artificial Sequence
  Oligonucleotide Alcvers walkup2
gccacaaaccggagcatcacagcgcct

SEQ ID NO: 44 - DNA, 20 bp, Artificial Sequence
  Sense PCR primer (Alcvers for)
ggttgctcgccatggatgac

SEQ ID NO: 45 - DNA, 20 bp, Artificial Sequence
  Antisense PCR primer (Alcvers rev)
ttcatggcatccggctaagc

SEQ ID NO: 46 - DNA, 2618 bp, Aspergillus versicolor
aattcgcccttggttgctcgccatggatgacccccgccgccgccagtttcatagttgtgacccctgtcgcaagggcaagaggcgctgtgatgctccggtttgtggccatctcccactctgctttttatcatcggctaattctgatatcaaatacttcttagagcaaccgggaaaatggtaactttgattcttgcactaactgcaagcgatggaagaaagagtgcacatttacctggctctcctcgaagccagcgaagcgtgcggaccccaaaggacgagcaagaccgaaaccgggcgtttcgactacttctagcaaacctagtgctgccagcaaccctagcactactagtaaccctagtagtgatagcggtgggacacctcctgatccaagtcgcgttgtcccttccatggtgggctcctataatgccctcgtggacgggggggcgtcatctgcttcgcaatggtatcctaccaaccccaatgatatgttcgcttcctcaaatattgtaccccatcctcatccttgcttccagggggcaccattattggagacggactggggccgagtgatggctcatccggtttattctcgtggaatatgagcgttccaaatgactggcaggtcagggatgtgactgaagagcctggtaattcgtttagtggactcgaacctcaagcagttttccctgatcctactctaccaaatgcccttgacaacacattcgatgtggtccaacaactacaagactcatcctacccttcctcttcctcttttgaattcacaccccccgattcatcaacggccgagtctaatcggcgggaaaagaaacaaaatcctcagtggagcttctgcctcgcttccgataatacagctgataaatatgctcgttcaacgatgacgcacaatttgatccgtatataccacgacagtatggagaacgcgttgtcatgctggttgacggagcacaactgcccttataccgataaaataagcagcctgctgccatttaatgaaagaaaggaatggggtcccagctggtcgaacaggatgtgcatccgggtctgtcggttggaccgtgcatcctcttcaatacgtggccgggcgttgagcgcggaagaggacaagaccgcagcccgggcactccacctggccatcatggcatttgcctcacagtggactcagcatgcgcaaagaggatcagatttatacgtccccgccccgatcgactatgacgagcgatccatccgtaaaaacgtttggaatgacgcgcgccacgccttagagcactcaacaaggataccctctttccgcattatattcgcaaacatcatattctcgttaacccagagtcccttggaccatagtcaagacgaacggctgggtcagctattggaaactgacagtgcgcctttctttcttgaaaccgccaatcgccagctttacaactttagacacaagttcgccagactccaacgggaggcacctccctctccaagtgtgagggagcttcggagggggtcggtagggtcgacaatgactgatgtactggagatgccgacgtcttctgcttctgagtctccccaggttgatccgattctcgatagccaggaccaccgcactactctcggtcttatgttctggctgggggtcatgtttgacaccttgagttctgcaatgtaccagcgaccattagtggtatcagatgaggacagccagattgcatcagcctcgcctccgatagccgaaccggaagagcaaatcgacttagactgctttaatatcccccaaagtggagtgcgtaaaaagcaggacgtatggggcgactttttcctccgcagttcccttgaacgccaggaatccacacaaatacagataagatggccatgctcctacgaagatgctgcggccgttctctccgaggcaacacccgtcaaagtcctgctttaccgccgcatcacacaactccaaaccctaatataccgaggggcgagtcctgaccgacttgaggaagccattcagaagactctcctagtttatcagcactggaactccatataccagggcttcatgctcgactgtgtcgctaaccacgaattcctccctcctcgtattcaatcgtggtacgtgattcttgacggccactggcatctcgccaccatgcttctagcagacattgtagaaagcatcgacaacggacggctcggttcgaagctcggccgcgaggctcgacaagccacagactttgtctcaaatctacgaattgataatgcattggcggtcggtgcccttgctcgttcatcactacacggacaagaccccgtcatgctccgctatttccacgattcccttaacgaggtggctttcctcgttgagccgtggacagttgttctcgtccattgtttcgccaaggcggcatctatctcgctggaaagcatacatgttatacctggcgagcccatggacgtattgtcggagagattccggcagaactgcgagttctgtatctgtgcgcttcagtatcttgcaaggaagtcggatatggctttcttggtgtcaaggaatttgtccaggtcgttggatctgaagcttagccggatgccatgaaaagggcgaattc

SEQ ID NO: 47 - DNA, 646 bp, Aspergillus versicolor
  misc_feature (644)..(644): Translation start site
gaatttcccaacgtcaatcaagagtttgttttaagtgctacggaatatatcaaagctctctcgtaaagcacaggtaatcccttcccatgcgacttcatcttcaagtttcagcaatttggaacacgatatgtccataattaaggaggcctgtggatgtggaagggttggaggaggccaccaatccggggatgtcgagcaacgatcagcattcgccaaatcaacgtacctctcgttaattagctctgattagtgtgatgagctcttatatcactccgccacccgctcgctctcgtccttcgtccccggcaactgctccayagacttggaagacrcctctcggctcggcaccgttctcgcccatcggttcaatccgccgactttgatgcttcaaatctcccaaagatccttggaaaatctatcttcgccctccagattgggcagcggaacgtatcgccgccataccggtaccccgaccccacactaggcttccccacccggaccccgcacagttcgtgayctccttgggaggagctgaagctgggtgcccctgcgacaagttatctgcgtcgggatcccgctttgtctcttcatctcctcggaacccaatgcagaagtcgttatcaaactcggttgctcgccatggatg

SEQ ID NO: 48 - DNA, 27 bp, Artificial Sequence
  Oligonucleotide flav walk3
cgaaggacgtggtgtgtcgatgccaac

SEQ ID NO: 49 - DNA, 27 bp, Artificial Sequence
  Oligonucleotide flav walk4
ttagcacacaattatgcgcactccatg

SEQ ID NO: 50 - DNA, 27 bp, Artificial Sequence
  Oligonucleotide Alcflav walk5
ttccgcgtagcctttgccaatgtattg

SEQ ID NO: 51 - DNA, 27 bp, Artificial Sequence
  Oligonucleotide Alcflav walk6
gaatggagctcgacgagctcttagatc

SEQ ID NO: 52 - DNA, 27 bp, Artificial Sequence
  Oligonucleotide Alcflav walk7
ctggactgtctctctcaccatacagag

SEQ ID NO: 53 - DNA, 27 bp, Artificial Sequence
  Oligonucleotide Alcflav walk8
gcgatgttgctcgcagataccgttgag

SEQ ID NO: 54 - DNA, 27 bp, Artificial Sequence
  Oligonucleotide Alcflav walkup1
ttgtgactcaagagccagtcgaacgtg

SEQ ID NO: 55 - DNA, 27 bp, Artificial Sequence
  Oligonucleotide Alcflav walkup2
agcagtgttggaattccgttcaagctc

SEQ ID NO: 56 - DNA, 21 bp, Artificial Sequence
  Sense PCR oligonucleotide (Alcflav for)
atgtcttatcgtcgccgtcag

SEQ ID NO: 57 - DNA, 22 bp, Artificial Sequence
  Antisense PCR oligonucleotide (Alcflav rev)
tcaaagggcgcacatatgatag

SEQ ID NO: 58 - DNA, 2505 bp, Aspergillus flavus
gaattcgcccttatgtcttatcgtcgccgtcagcatcgtagttgtgatcaatgtcgtaaaggcaagagagcatgcgatgccctcctggctgacgagcttgaacggaattccaacactgctgctcgacaagcgtacaatcacgcgtgctccaattgcagaaaatacaaaagaaaatgcacgttcgactggctcttgagtcacaaggaatcccggcatgctcatagcaagagagccagaaatatcgcgatcgccctctcgcggcaggtgaacgattgttccgctcattcctctcaacaaacctccactgggcgcaatcctacagagctccctctgcaaaacatcgaggattgcgaatggccaacgtctgttagggacccgcttttgccgttcccacaagacgaggaactagatgcggactggttaacttggggatgcctcaacgacgcagtgtccatctctcctctaagcgccgacatgactctcaatggggataggcacgtcaatcctaaccagacaccacaaatgagtactcaatggaactctgtcggggccggccaggcatggcaaagtatcggtcaaacttcactgctcgacacgatgaacagttctataacttcgtcgcaattcaaggatacacccgactatcgatcatttgagacatgggatatcagttctgagctcccgcttcacggtcttccacctaccgaaggacgtggtgtgtcgatgccaacaaacactacactgtgtgtgggctcaaaccaattagcacacaattatgcgcactccatgatgacgcgcaacctaattcacatttataacgacagtatggaaaatgcattgagctgttggctgaccgagcgtaattgtccctacagtgcccgggggtacgttgacaaaacagggccgaagacaggtccttataccacgaataggatctacagacgaatttgcctcttggatagggcatgctcatccatcccgggtcgacgtctcacgagtgtggagagtagaacagcaacacagacacttcatgctgtcattatggcattcgcttctcagtggctggagaggccttcagcagacaaagatatcccaataccatcttcttcagctcaccacgaaagtggcatgcgtgagggtctctggaatgaagcgcgtcatgcgctggagaattcgagagcaattccatcgttccgcgtagcctttgccaatgtattgttttcgctggcgcaacgacccctacacgttgaagaaggaatggagctcgacgagctcttagatcacgatcctgccccaatgtatctcgaaacggggcttaggcagctgtttacttttcgttctagattgattaagcttcggcggcaaggtcccaaccgagcgctcgagcaatgctgcaaggagagcaaaggggataaaagcacccatcagttgagccaaatcgatctgatgctgaaggactctgaaacccatcacactttcgatcttctattctggctgggcatcatgtttgatacgttgacagctgtcatatatcaacgtcccccggtcatttccgatgaggacagtcagatcatacgcccccggtcacgcttctcgtttccggacgccgttgatctggatggatgggatattagctcctattccgctagccgacgtgaagaaagtgtatggggcgatctttttcttcgcaaacgtaacatgctccacaatctcaaccaggcccgctggccttgttcttacgaggaggcagcagaagtcttgtccgacgccgcgccagtcaaggttctcctattccgtcgcataaatcatatcaataccctggtatgccggggaggtggggcagaggccattgaagaagcgatccacagcgctctcttggtctacgagtattggaactcctcgtacaagcagtttatgctggactgtctctctcaccatacagagctcccgtctcgcatacaatcatggtatctagtgctggctggacattggcatcttgcggcgatgttgctcgcagataccgttgaggagatagatcaagcccgacttggtcaaaactcccaaaccgaacatcggtataccacaggcctcatctccgtcttgcgtcacgaaaatgctttcgctgttggagggctcgcccaatactcttacgacctgcagggctcgtcgcaccctaaactccgcaattttcacgattcagtcaatcaagcggcatccttgactgagccatggactgctgtccttatccactcttttagaaaagcaggtactatcctaatcagagagattggcagattacaatgtggttaccaaatgcagcaggaatctttcatgctggcgtatcagcgttgtgaacactgtataaaggcactccagtgcctgggaagaaagtcagatatggctctggccgcagctcagagtctatcagacagtctcaacatgacactgttgcgacccagtcctattgattcctatcatatgtgcgccctttgaaagggcgaattc

SEQ ID NO: 59 - protein, 826 aa, Aspergillus flavus
MSYRRRQHRSCDQCRKGKRACDALLADELERNSNTAARQAYNHACSNCRKYKRKCTFDWLLSHKESRHAHSKRARNIAIALSRQVNDCSAHSSQQTSTGRNPTELPLQNIEDCEWPTSVRDPLLPFPQDEELDADWLTWGCLNDAVSISPLSADMTLNGDRHVNPNQTPQMSTQWNSVGAGQAWQSIGQTSLLDTMNSSITSSQFKDTPDYRSFETWDISSELPLHGLPPTEGRGVSMPTNTTLCVGSNQLAHNYAHSMMTRNLIHIYNDSMENALSCWLTERNCPYSARGYVDKTGPKTGPYTTNRIYRRICLLDRACSSIPGRRLTSVESRTATQTLHAVIMAFASQWLERPSADKDIPIPSSSAHHESGMREGLWNEARHALENSRAIPSFRVAFANVLFSLAQRPLHVEEGMELDELLDHDPAPMYLETGLRQLFTFRSRLIKLRRQGPNRALEQCCKESKGDKSTHQLSQIDLMLKDSETHHTFDLLFWLGIMFDTLTAVIYQRPPVISDEDSQIIRPRSRFSFPDAVDLDGWDISSYSASRREESVWGDLFLRKRNMLHNLNQARWPCSYEEAAEVLSDAAPVKVLLFRRINHINTLVCRGGGAEAIEEAIHSALLVYEYWNSSYKQFMLDCLSHHTELPSRIQSWYLVLAGHWHLAAMLLADTVEEIDQARLGQNSQTEHRYTTGLISVLRHENAFAVGGLAQYSYDLQGSSHPKLRNFHDSVNQAASLTEPWTAVLIHSFRKAGTILIREIGRLQCGYQMQQESFMLAYQRCEHCIKALQCLGRKSDMALAAAQSLSDSLNMTLLRPSPIDSYHMCAL

SEQ ID NO: 60 - DNA, 418 bp, Aspergillus flavus
  misc_feature (416)..(416): Translation start site
attcgcccttactatagggcacgcgtggtcgacggcccgggctggtatcatcaaacgctgaagtgggtggacgtttggaggcaatgcttggtgttccactgtcccatgacctctaataacctttggtagtttgcaatccatgactgatcaggttttctggagtcttcattgtagcatcccggccacaaagaacaagtcgtagccagtgggatttgacaggctgaaagtgacctcaagcgtaggcatatcacgaactattaatttaaaagtaaccccgacccgatctataccccgcaaacccccgcatttccccagcttagtccgtactttattatctctcggatccatgttcacctgaactattctcccagaaacggcctaccttgctgtcgactataacacattgcygcaaattatg

SEQ ID NO: 61 - DNA, 2489 bp, Aspergillus ustus
gaattcgcccttctcgaatgaagatgggagactcccgtcgccgccagaatcatagctggatccgtgtcgcaaggggaaacgagggtgtgatgcgcctgaaaatcgaagtggagatggaacacctgctcgaattgcaagcggtggaagaagaaatgcacattcaatttcgtctcgtccaggcgcgcagattcccgcgtcgtcggtgccaatgcccggtcaaaagcgaagtccacctctacccctgctgtctctaccgctgcatcggtagccacttctgcggctgcccctcccactcccgatatggcgacatccctgccatgctaaacacgggtatggacatgggcacgaatgagtacgatgctctccttcatgacggtttgcggtcgtcacaccttgaccctacgaggcttggggatatgtttgcttttacctcgccgtctagtttcacggcggaggctttgcatgcgcagagtgctgttggcacagaagccatcgcgtgggattcagggattccaacagactggtctatcccttcgatgcctcgtcggaaaagtcgttcactccgcttgagagtcaggcggtctttcttgcacaggaggattcgaaccagtttgacgttattcaggagttggaagatggctcatccgacaacttcacaccaccggggcggaaacgcgacgaggataagcgacggaaatttcaatgggagttatgcatcgcttccgacaaaacagccaaccaggttggccgatcgacaatgacgcgcaatctaatgcggatatatcacgatagcatggagaatgcgctctcatgttggttgaccgagcacaactgtccgtatgccgacccgatgagcgcaatgctaccttttaaccagaggaaagaatggggtcccagttggtcgaacaggatgtgtatccgggtctgtcatttagatcgggaatcatcctcgatacgcggaagggcactgagcgtagacgaggaccggacggccgcgcgggcgctgcatctcgcaattgtcgcattcgcctcacagtggacgcagcatgcccaaagggggacagggctttcggttccgactgatatcgcgtacgatgaacggtcgattcgaaagaatatatggaacgaggcgcggcatgctctacaacactccacagggatcccgtctttccgggtaatattcgccaacattattttctcattgacacagagtccgctggacgagaatcgacctgcgaagctaggtcagctgttggagaatgatggtgctcccgtattcctagagaacgccaatcgtcagctctacacattccgacacaagttcgcgagactccaacgagaggctcccccgcctgtggctgggctgcgacgaggttcaatatcatccactctcactgacgtgctggaagttccgactcctgaatctccacaggtcgatccaattctcgcgaatcaagaccaccgaagcacactcagcctcctcttctggcttggaatcatgttcgacaccctcagtgcagccatgtaccagcgccctcttgtcgtctcagacgaagatagccaaatcgcctccgcctccccgtcggcctcaaccaacccccgagtcaacctcaactattgggaaatcccagacagcaatctcccagcgaaaaacgacgtctggggtgaatttttccttcaacctgccgctcgccaggaactggcctccgcacatccccaaatccaaccaaaacaaccccgttggccgtgttcctacgaagaagccgcatcagtcctgtccgaggcaacaccggtaaaagtccttctctaccgccgcgtcacccaactccaaacccttatctaccgtggcgcgtctcccgcacggcttgaagaagtcattcaaagaacgcttctcgtctaccaccattggacctgcacatatcaatcatttatgctcgactgtgtggcaaaccacgagtcccttccacaccgtattcagtcttggtatgttattcttgatggccattggcacctctccgcaatgcttctcgccgatgtgctagagtccatcgacagaagccacctcggactcgagtcggagcgcgagtcccggattgcaagcgatcttattgcaacactgcgaatcgacaatgcactcgcagtcggtgccttggctagggcatcgctacacggggagaatagcatgatgcatcgacatttccatgactcgttgaacgaggtcgcgttcctggttgagccgtggacagtcgttttggtccattgtttcgcgaaggcggcggctatttcgctggattgtctgggtcagggacagggaggtgctttggcagaatgttttcggcagaattgtgaatattgtatttgtgcgttgaagtatttgggacggaaatcggacatggcgttttgtgttgcgggcgggttggagaaggagttgcttgagaaagctgggagtatgctgtcagcggatatccttgtgtaaaagggcgaattcc

SEQ ID NO: 62 - protein, 820 aa, Aspergillus ustus
MKMGDSRRRQNHSCDPCRKGKRGCDAPENRSGDGYTCSNCKRWKKKCTFNFVSSRRADSRVVGANARSKAKSTSTPAVSTAASVATSAAAPPTPDSGDIPAMLNTGMDMGTNEYDALLHDGLRSSHLDPTRLGDMFAFTSPSSFTAEALHAQSAVGTEAIAWDSGIPTDWSIPSMPRSEKSFTPLESQAVFLAQEDSNQFDVIQELEDGSSDNFTPPGRKRDEDKRRKFQWELCIASDKTANQVGRSTMTRNLMRIYHDSMENALSCWLTEHNCPYADPMSAMLPFNQRKEWGPSWSNRMCIRVCHLDRESSSIRGRALSVDEDRTAARALHLAIVAFASQWTQHAQRGTGLSVPTDIAYDERSIRKNIWNEARHALQHSTGIPSFRVIFANIIFSLTQSPLDENRPAKLGQLLENDGAPVFLENANRQLYTFRHKFARLQREAPPPVAGLRRGSISSTLTDVLEVPTPESPQVDPILANQDHRSTLSLLFWLGIMFDTLSAAMYQRPLVVSDEDSQIASASPSASTNPRVNLNYWEIPDSNLPAKNDVWGEFFLQPAARQELASAHPQIQPKQPRWPCSYEEAASVLSEATPVKVLLYRRVTQLQTLIYRGASPARLEEVIQRTLLVYHHWTCTYQSFMLDCVANHESLPHRIQSWYVILDGHWHLSAMLLADVLESIDRSHLGLESERESRIASDLIATLRIDNALAVGALARASLHGENSMMHRHFHDSLNEVAFLVEPWTVVLVHCFAKAAAISLDCLGQGQGGALAECFRQNCEYCICALKYLGRKSDMAFCVAGGLEKELLEKAGSMLSADILV

SEQ ID NO: 63 - DNA, 20 bp, Artificial Sequence
  Oligonucleotide Alcvers seq4r
caaattgtgcgtcatcgttg

SEQ ID NO: 64 - DNA, 19 bp, Artificial Sequence
  Antisense PCR oligonucleotide (Alcvers seq5r)
ggaagcgaacatatcattg

SEQ ID NO: 65 - DNA, 2529 bp, Aspergillus versicolor
atggatgacccccgccgccgccagtttcatagttgtgacccctgtcgcaagggcaagaggcgctgtgatgctccgagcaaccgggaaaatggtaactttgattcttgcactaactgcaagcgatggaagaaagagtgcacatttacctggctctcctcgaagccagcgaagcgtgcggaccccaaaggacgagcaagaccgaaaccgggcgtttcgactacttctagcaaacctagtgctgccagcaaccctagcactactagtaaccctagtagtgatagcggtgggacacctcctgatccaagtcgcgttgtcccttccatggtgggctcctataatgccctcgtggacgggggggcgtcactgcttcgcaatggtatcctaccaaccccaatgatatgttcgcttcctcaaatattgtacccccatcctcatccttgcttccagggggcaccattattggagacggactggggccgagtgatggctcatccggtttattctcgtggaatatgagcgttccaaatgactggcaggtcagggatgtgactgaagagcctggtaattcgtttagtggactcgaacctcaagcagttttccctgatcctactctaccaaatgcccttgacaacacattcgatgtggtccaacaactacaagactcatcctacccttcctcttcctcttttgaattcacacccccgattcatcaacggccgagtctaatcggcgggaaaagaaacaaaatcctcagtggagcttctgcctcgcttccgataatacagctgataaatatgctcgttcaacgatgacgcacaatttgatccgtatataccacgacagtatggagaacgcgttgtcatgctggttgacggagcacaactgcccttataccgataaaataagcagcctgctgccatttaatgaaagaaaggaatggggtcccagctggtcgaacaggatgtgcatccgggtctgtcggttggaccgtgcatcctcttcaatacgtggccgggcgttgagcgcggaagaggacaagaccgcagcccgggcactccacctggccatcatggcatttgcctcacagtggactcagcatgcgcaaagaggatcagatttatacgtccccgccccgatcgactatgacgagcgatcatccgtaaaaacgtttggaatgacgcgcgccacgccttagagcactcaacaaggataccctctttccgcattatattcgcaaacatcatattctcgttaacccagagtcccttggaccatagtcaagacgaacggctgggtcagctattggaaactgacagtgcgcctttctttcttgaaaccgccaatcgccagctttacaactttagacacaagttcgccagactccaacgggaggcacctccctctccaagtgtgagggagcttcggagggggtcggtagggtcgacaatgactgatgtactggagatgccgacgtcttctgcttctgagtctccccaggttgatccgattctcgatagccagaccaccgcactactctcggtcttatgttctggctgggggtcatgtttgacaccttgagttctgcaatgtaccagcgaccattagtggtatcagatgaggacagccagattgcatcagcctcgcctccgatagccgaaccggaagagcaaatcgacttagactgctttaatatcccccaaagtggagtgcgtaaaaagcaggacgtatggggcgactttttcctccgcagttcccttgaacgccaggaatccacacaaatacagataagatggccatgctcctacgaagatgctgcggccgttctctccgaggcaacacccgtcaaagtcctgctttaccgccgcatcacacaactccaaaccctaatataccgaggggcgagtcctgaccgacttgaggaagccattcagaagactctcctagtttatcagcactggaactccatataccagggcttcatgctcgactgtgtcgctaaccacgaattcctccctcctcgtattcaatcgtggtacgtgattcttgacggccactggcatctcgccaccatgcttctagcagacattgtagaaagcatcgacaacggacggctcggttcgaagctcggccgcgaggctcgacaagccacagactttgtctcaaatctacgaattgataatgcattggcggtcggtgcccttgctcgttcatcactacacggacaagaccccgtcatgctccgctatttccacgattcccttaacgaggtggctttcctcgttgagccgtggacagttgttctcgtccattgtttcgccaaggcggcatctatctcgctggaaagcatacatgttatacctggcgagcccatggacgtattgtcggagagattccggcagaactgcgagttctgtatctgtgcgcttcagtatcttgcaaggaagtcggatatggctttcttggtgtcaaggaatttgtccaggtcgttggatctgaagcttagccggatgccatgaaaagggcgaattc

SEQ ID NO: 66 - protein, 839 aa, Aspergillus versicolor
MDDPRRRQFHSCDPCRKGKRRCDAPSNRENGNFDSCTNCKRWKKECTFTWLSSKPAKRADPKGRARPKPGVSTTSSKPSAASNPSTTSNPSSDSGGTPPDPSRVVPSMVGSYNALVDGGASSASQWYPTNPNDMFASSNIVPPSSSLLPGGTIIGDGLGPSDGSSGLFSWNMSVPNDWQVRDVTEEPGNSFSGLEPQAVFPDPTLPNALDNTFDVVQQLQDSSYPSSSSFEFTPPDSSTAESNRREKKQNPQWSFCLASDNTADKYARSTMTHNLIRIYHDSMENALSCWLTEHNCPYTDKISSLLPFNERKEWGPSWSNRMCIRVCRLDRASSSIRGRALSAEEDKTAARALHLAIMAFASQWTQHAQRGSDLYVPAPIDYDERSIRKNVWNDARHALEHSTRIPSFRIIFANIIFSLTQSPLDHSQDERLGQLLETDSAPFFLETANRQLYNFRHKFARLQREAPPSPSVRELRRGSVGSTMTDVLEMPTSSASESPQVDPILDSQDHRTTLGLMFWLGVMFDTLSSAMYQRPLVVSDEDSQIASASPPIAEPEEQIDLDCFNIPQSGVRKKQDVWGDFFLRSSLERQESTQIQIRWPCSYEDAAAVLSEATPVKVLLYRRITQLQTLIYRGASPDRLEEAIQKTLLVYQHWNSIYQGFMLDCVANHEFLPPRIQSWYVILDGHWHLATMLLADIVESIDNGRLGSKLGREARQATDFVSNLRIDNALAVGALARSSLHGQDPVMLRYFHDSLNEVAFLVEPWTVVLVHCFAKAASISLESIHVIPGEPMDVLSERFRQNCEFCICALQYLARKSDMAFLVSRNLSRSLDLKLSRMP

SEQ ID NO: 67 - DNA, 21 bp, Artificial Sequence
  Degenerate oligonucleotide n-alcr2
atggmwgaymcgcgccgmcgc

SEQ ID NO: 68 - DNA, 23 bp, Artificial Sequence
  Degenerate oligonucleotide c-alcR
aasaaacgcatatccgacttcct

SEQ ID NO: 69 - DNA, 19 bp, Artificial Sequence
  Degenerate oligonucleotide AlcRATG
atggcagatacgcgccgac

SEQ ID NO: 70 - DNA, 24 bp, Artificial Sequence
  Degenerate oligonucleotide alcRTGA
ctacaaaaagctgtcaactttccc

SEQ ID NO: 71 - DNA, 23 bp, Artificial Sequence
  Degenerate oligonucleotide alcMID
tccgacataagtttgcacgaatg

SEQ ID NO: 72 - DNA, 23 bp, Artificial Sequence
  Degenerate oligonucleotide alcMIDR
cattcgtgcaaacttatgtcgga

SEQ ID NO: 73 - DNA, 2384 bp, Artificial Sequence
  PCR generated sequence using consensus oligonucleotide of A. bicolor
ttgtgatccgtgtcggaaggggaagagggggtgcgatggtcagcactagaatttcatctatgtcttgaagaatcaagctaataagtgtaggaaaccggactgaaattctcttcaattcctgctcgaactgcaaaaaatggaaaaaggagtgcgcgttcaactggctggccacaaatcccactatcaaaggcaagggaaaccaggaaaagaacaggagaactaaagctaagcctagtactgccgcgactgatacaaatacggctattgctacgcctgatgatagtgtcgacatcccttctgttggcagtgatgttggtatcagcgtgggcgatggctcctacggtagttgtatcgatgatggacttcagtctgcgcagtggtttcctgttaatcccggcaacggtgatgtgctcgcgctgcctgggactggattgtttgaccttacttcgtcttcattgttgtttccagaagggggtatcggggaaacgatacgagtgacccatatgcacagtctataatttcgtggaacatgggcgggctttcctgacaattggcaacttggtgctgtacctggaaagtctttcgccagacttgacctacctacaaactcgctcgatgacacattcgacataatccaaccactcgaagaagattcaagccgaaattcgaggtaattcccatccggcttctgcatcgcctccgacaacacggccaaagcctacgctcgctcaacaatgacacgcaaccttctccgcatataccacggcagcatggataacgcactatcatgctggctaacagagcataactgcccgtacattgactcaatcgggcgaccttctactactatacagccaaagaaaggaatggggcccgaactggtcaaatcgcatgtgcatctgagtttgccaattagatcgcgcatcctcttcaattcgcagtagggcattgagcgcagaagaggacatgactatggtatttgcctcgcagtggactcagcatgcgcaacggggaccggtcctatctgtccctgcgggaattgatgaaaatgagagatcaattaggaagaatgtctgggatgagatacgccatgcgcaagagcattcaacgaggattccctcgttccgggtgatttatgcgatttgcgaatatcatcttctcgttgacgcagagcccgctagacaaaggcgaggcgaggttaaggactgggtcagctactagagaattacagtgcaccgatattcctcgagaacaccaacagacagcgataccccttccgacataagttcaccaggctccagcgacgtaatcggagctcgccacaagtcgaccccatcctatccagtcaggaccaccgcggtacgctgaacctgctcttctggttcggaatcatgttcgacacgctaagtgcagcaatgtatcaacgccctctcgttgtctcagacgaggatagtcaaatcgcatcaatctcacctccccctcccaccccctctccactcaaccccccagcccaaaataacctcgagtgctggaacttcccctcagaccaaccacagaccacaacgctaaccatccgctgaaaacaagacgtctggggctacagcttcctccacccaacagcctccctctcacaccaagaacccaccacccagctcaaccctcacctcagccaaaacaccgccccaaacgctggccctgtacatacgccgaatcagcctcgattctctccttcgcaacccccgtaaaagtcctcctctaccggcgcgtcacccaactccaaaccctcatctaccgcggcgcagcaccctcgcaactcgaatccgtcatccagaagacactcctcgtctacaaccattggcagcaattctacgcgcccttcatgacagactacgtaaccaaccacgctattctcccgccgagaattcactcctggtgtgtcatgttagacggccattggcatctcgctgcgatgctattagccgttgtagttgaggagactgataacgccgggcttgggttagactctgcgcgagaggcaagaaacttatcggatttcgtcgggacattaaggagggagaacgccttagccgttggcgcgctcgcgagggcaccattgcagggccagaatccgggtatggaagaacattaccataatagtttgaacgaggttgcgtttccggtggagccgtgggcggctgttctggtatattgttttgcgaaggggggggggggttgtatattccgcttgagagggtgggttattcgtcgtttactagggatgggtctggggatggcgttaaggacgggaaggtatttcggcttaattgtgagctttgtatttgtgtttcagagtatcttggaaggaagtcggatatgcgtttgtt

SEQ ID NO: 74 - DNA, 2466 bp, Artificial Sequence
  PCR sequence generated using consensus oligonucleotides of A. corrugatus
atggatgacacgcgccgccgccagaatcatagctgcgacccctgtcgcaagggcaagcgacgctgtgatgccccggaaaataggaacgaggccaatgaaaacggctgggtttcgtgctcaaattgcaagcgttggaacaaggattgtaccttcaattggctctcatcccaacgctccaagccaaaaggggctgcgcccagggcgaggacgaagaaggccagaactgctacaaccaccagtgaaccatcaacttcagctgcagcaatccctacaccggaaagtgacaatcacgatgcgcctccagtcatcaacgctcacgacccgctcccgagctggacgcaggggctgctctcccaccccggcgacctttttgattttagccagtcgtctattcccgcaaatgcagaagatgcagccaacgtacagtcagacgcaccttttctgtgggatctagccatacccggtgatttcagcataggccaacagctcgagaaaccactcagtccgctcagttttcaagcagttcttctcccgccccatagcccgaacacggacgacctcattcgcgagctggaagagcagactacggatccggactcggtcaccgatactaatagtctacaacaggtcgctcaagatgggtcgcgatggtctgatcggcagtcgcagctactacctgagaacagtctgtgcatggcctcagacagcacagcacggcgatatgcccgtacctcaatgacgaagaatctgatgcgaatctaccacgatagtatggagaatgcactgtcctgctggctgacagagcacaactgtccatactccgaccagatcagctacctgccgcccaagcagagggcggaatggggcccgaactggtcaaacaggatgtgcatccgggtgtgccggttagaccgtgtatccacctcattacgtgggcgcgccttgagcgctgaagaggatagagccgcggcacgagccctgcacctggctatcgtagcctttgcgtcgcaatggacgcagcatgcgcagaggggggctgggctatctgttcctgcagacatagcgggcgatgagagggccatccggaggaacgcctggaatgaagcacgccatgccttgcagcacacgactggaattccgtcgttccgggttatatttgcgaatatcatcttttctctcacgcagagtgtgctggatgatactgagcagcagaatgtgggtgcacgtctggacaggctactcgagaatgacggtgcgcccgtctttctggaaaccgcgaaccgtcagctttatacattccgacataagtttgcacgaatgcaacgccgcggtaaggctttcaacaggctcccggtggaatctgtcgcatcgacattcgccgatactttcgagacaccgacgccgccgtctgaaagcccccagcttgacccggttgtggccagtgaggagcatcgcagtacattaagccttatgttctggctggggatcatgtttgatactctcagcgctgcaatgtaccagcgaccactggtggtgtcagatgaggatagccagatatcatcggcatatccatcaacgcgcggatctgaaacgccaatcaacctagactgctgggaaccaccgagacaggccccgagcaatcaagaaaaaagcgacgtatggggcgacctcttcctccgcacctcggactctctccaaggtcacgaatcccacacacaaatctcccagccagcggctcgatggccctgcacctacgaacaggccgccgccgctctctcctctgcaacgccagtcaaagtcctcctctaccgccgcgtcacgcagctccaaaccctcctctatcgcggcgccagccctgcccgccttgaagcggccatccagagaacgctccacgtctataatcattggacagcaaagtaccaaccatttatgcaggactgcgttgctaaccacgagctccttccttcacgcatccagtcttggtacgtcattctagacggtcactggcatctagccgcgatgttactagcggacgttttggagagcatcgaccgcgatgcgtactctgatatcaaccacatcgacctcgtcacgaagctaaggctcgataatgcactggcagttagtgcccttgcgcgctcttcactccgaggccaggagctagatccgggcaaagcatctccgatgtatcgccatttccatgattctctgaccgaggtggcattcctggtagaaccgtggaccgtcgttcttattcactcattcgccaaggctgcgtatatcttgctggactgtttagatctggacggccagggaaatgcactagcggggtacctgcaactgcggcaaaattgcaactactgcgttcgggcgctgcagtttctgggcagaaagtcggatatggcggcgctggttgcgaaggatttagagagaggtttgaatgggaaagttgacagctttttgtag

SEQ ID NO: 75 - DNA, 2453 bp, Artificial Sequence
  PCR sequence generated using consensus oligonucleotides of A. cleistominutus
gccctttgtgatccctgtcgcaagggcaagcgacgatgtgatgccccggtaggttgccgatatcggatccgcagcgtctgcaccgacagtcgctgagatgtaacacaggaaaatagaaacgaggccaatgagaacagctgggtttcttgctcaaattgcaagcgttggaacaaggattgtaccttcaattggctctcgtcccagcgctccaagccaaaaggagctgcgccccgagccaggacgaagaaagccagggccgctacaaccaccagtgaaccatcaacttcagctgcagctttccctacaccggaaagtgacaatcacgatgcgcctccagtcatcaacgctcatgacgcgctcccgagctggactcaggggctgctctcccaccccagcgaccttttcgatttcagccagtcctctattcccgcaaatgtagaagatgcagcagccaacgtgcagtcagacgcaccttttccgtgggatctggccatccccggtgatttcagcatgggccaacagcttgagaaaccactcagtccgctcagttttcaagcagttcttctcccgccccatagcccgaacacggatgacctcattcgcgagctggaagagcagacaacggatccggactcggttaccgatactaatagtctgcaacaggccgctcaacatgggtcgctatggtctgatcggcactcgccactgctaccagagaacagtctgtgcatggcctcagacagcacagcacggcgatatgcccgttcctcaatgacgaagaatctgatgcgaatctaccacgatagtatggagaatgcactgtcctgctggctgacagagcacaattgcccatactccgaccagatcagctacctgccgcccaagcagagggcggaatggggcccgaactggtcaaacaggatgtgcatccgggtgtgccggttagaccgcgtatccacctcattacgcgggcgcgccttgagcgccgaagaggacagagccgcagcccgagccctgcatctggcgatcgtagcctttgcatcgcaatggacgcaacatgcgcagaggggggctgagctatctgttcctgcagacatagcggccgatgagagggccatccggaggaacgcttggaatgaagcacgccatgccttgcagcacacgacagggattccctcgttccgggttatatttgcgaatatcatcttttctctcacacagagtgtgctggatgatactgagcagcagggtgtgggtgcccgtctggacaggctactcgagaatgacggtgcgcccgtctttctggaaaccgcgaaccgtcagctttatacattccggcataagtttgcacggatgcaacgccgcggtaaggctttcaacaggctcccggggggatctgtcgcatcgacattcgcggatattttcgagacaccgacaccgtcgtctgaaagcccccagcttgacccggttgtggccagtgaggagcatcgcagtacattaagccttatgttttggctagggatcatgttcgataccctaagcgctgcaatgtaccagcgaccactcgtggtgtcagatgaggatagccagatatcatcggcatctccatcaacgcgcggctctgaaacgccaatcaacctagactgctgggaaccaccgagacaggtcccgagcaaccaggacaaaagcgacgtatggggcgacctcttcctccgcgcctccgactctctccaagatcacgaatcccacacacaaatctcccagccagcggctcgatggccctgcacctacgaacaggccgccgccgcgctctcctctgcaacgcccgtcaaagtcctcctctaccgccgcgtcacgcagctccaaaccctcctctaccgcggcgccagccctgcccgccttgaagcggccatacagagaacgctccacgtctataatcactggacagcaaagtaccaaccatttatgcaggactgcgttactaaccacgagctcctcccttcgcgcatccagtcctggtacgtcattctagacggtcactggcatctagccgcgatgttgctagcggacgttttggagagcatcgaccgcgattcgtactctgatatcaaccacatcgacctcgtcacaaagctaaggctcgataacgcactggcagttagtgcccttgcgcgctcttcactccgaggccaggagctagacccgggcaaagcatctccgatgtatcgccatttccatgattctctgaccgaggtggcattcctggtagaaccgtggaccgtcgttcttattcactcgttcgccaaggctgcgtatatcttgctggactgtttaaatctggacagtcagggaaatgcacttgcggggtacctgcagctgcggcaaaattgccactgctgcattcgggccctgcagtttctgggcaggaagtcggatatgcgtttgttaagggc

SEQ ID NO: 76 - DNA, 2380 bp, Artificial Sequence
  PCR sequence generated using consensus oligonucleotides of A. faveolatus
tgtgacccctgtcgcaagggcaagcgacgctgtgatgccccggaaaatagaaacgaggccaatgaaaacggctgggtttcgtgctcaaattgcaagcgttggaacaaggattgtaccttcaattggctctcatcccaacgctccaagccaaaaggggctgcacccagggcgaggacgaagaaatccaggaccgctacaaccaccagtgaaccagcaacttcagctgcagcaatccctacaccggaaagtgacaatcacgatgcgcctccagtcatcaacgctcacgacgcgctcccgagctggactcaggggctgctctcccaccccggcgaccttttcgattttagtcactctgctattcccgcaaatgcagaagatgcagccaacgtgcagtcagacgcaccttttccgtgggatctagccgtccctggtgatttcagcatggtccaacagctcgagaaaccactcagtccgctcagttttcaagcagttcttctcccgccccatagcccgaacacggatgacctcattcgcgagctggaagagcagactacggatccggactcggttaccgatactaatagtctacaacaagtcgctcaagatggatcgctatggtctgatcggcagtcgccgctactacctgagaacagtctgtgcatggcctcagacagcacagcacggcgatatgcccgttcctcaatgacgaagaatctgatgcgcatctaccacgatagtatggagaatgcactgtcctgctggctgacagagcacaattgtccatactccgaccagatcagctacctgccgcccaagcagagggcggaatggggcccgaactggtcaaacaggatgtgcatccgggtgtgccggttagatcgcgtatctacctcattacgcgggcgcgccttgagcgccgaagaggacagagccgcagcccgagccctgcatctggcgatcgtagcttttgcttcgcaatggacgcagcatgcgcagaggggggctgggctatctgttcctgcagacatagcggccgatgagagggccatccggaggaacgcctggaatgaagcacgccatgccttgcagcatacgacggggattccgtcgttccgggttatatttgcgaatatcatcttttctctcacacagagtgtgatggatgataatgagcagcagggtgtgggtgcacgtctggacaagctactcgaaaatgacggtgcgcccgtgttcctagagaccgcgaaccgtcagctttatacattccggcataagtttacacggatgcaacgccgcggtaaggctttcaacaggctcccggggggatctgtcgcatcgacattcgccgatattttcgaaacaccgacgctgtcgtctgaaagcccccagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccatcaacgcgcggctctgaaacgccaatcaacctagactgctgggaaccaccgagacaggttccgagcaatcatgaaaacagcgacgtatggggcgacctcttcctccgcacctcgggctctctccaagagcacgaatcccacacacaaatctcccagccagcggctcgatggccatgcacctacgaacaggccgccgccgctctctcctctgcaacgcctgtcaaagtcctcctctaccgccgcgtcacgcagctccaaaccctcctctatcgcggcgccagccctgcccgccttgaagcggctatccagagaacgcttcacgtctataatcactggacagcgaagtatcaaccatttatgcaggactgtgttgctaaccacgagctccttccttcgcgcatccagtcctggtacgtcattttagatggtcactggcatctagccgcgatgttgctagcggacgttttggagagcatcgaccgcgattcgtactctgataccaaccacatcgacctcgtcacaaaactaaggctcgataatgcactggcagttagtgcccttgcgcgctcttcactccgaggccaggagctagacccgggcaaagcatctccaatgtatcgccatttccatgattctctgactgaggtggcattcctggtagaaccgtggaccgtcgttcttattcactcgtttgccaaggctgcgtatatcttgttggactgtttggatctggacggccagggaaatgcactagcgggttacctgcagctgcggcaaaattgcaactactgcattcgggcgctgcagtttctgggcaggaagtcggatatgcgtttgttaa

SEQ ID NO: 77 - DNA, 2448 bp, Artificial Sequence
  PCR sequence generated using consensus oligonucleotides of A. heterothallicus
tgtgatccgtgtcggaaggggaagagagggtgtgatgcgcttgtgagttgtgtcgtgcctgtctaactgcttgacctgccaggatcatgccataccagatcccgagctcgtcggagtccaaacctttctaaccatgatccaggagattcgaagtggagatggatatacgtgctcgaattgcaaacgatggaagaagaagtgcacttttaatttcgtctcgtcgaggcgcgcagacgcccgtagtgtcgctgccaattctcgggcaaaagcgaagcccacttcgacccctgtcgtcgctaccactgcatcggtagctacttctgtagtggcccctccaacgccagatagtggcaacatccctgctatgctgaatatgggcatcaatacaagtgagtataatgcactgcttgacgaggggttgcgatcgtcgcagcttgacccggcaagattcggagacatgtttgaattcatgtcgccgtcgaactttgctgcggaggtgttgcatgcgcagagcgctattgggggagtgaacgagacgctcgcgtggactatgggggttccaggaagttggccgatgggcatgatgccgcaatcagaaacgtctttgagttcacttcaatcgcaggagctattcatttcgaacgaggacgcgaacccgtacgatgttatccaacagttggaagacgatttcgaggatcctgcgacatcggtcagcaaacgcgacgaagatgtgcgaaagttccagtgggagttatgtatcgcgtcagacaaaacagccaacaaggtcggccgttcgacgatgaatggaaatttgatccgaatataccacgacagcatggaaaacgcgctgtcatgttggctaaccgaacacaactgtccgtatgccgacccgatgagcgccatgttaccgttcaatcaaagaaaagaatggggtccaagttggtccaatagaatgtgcattcgggtttgtcggttagatcgtgcatcctcgtcaatacgtgggagagcattgagcgtagaggaagataggactgcggcacgggcccttcatctcgcaattgttgccttcgcctcacaatggacgcaacatgcgcagaaaggaacgggtttatcagttccggcaggcatcgcatatgacgagcggtcgactcgcaaaaatatctggaacgaggcgcggcacgcgttgcaacattcaactggtattccgtcattcagggtggtatttgccaacatcattttctcccttacgcagagtccgctggacgagactcggcctgcaaagttggcgcagctattagacaacgacggcgcgcctgtgtttctagaaaatgcgaaccgtcagctttacacatttcggcataaatttgcaagactacagcgcgaagctcctccacctgccgcgacagacctccgacgaggttcgatatcatccacactcaccgaggtgctggagattccgactccagaaagtccgcaacttgaccccatcctcgccagccaagaccatcgcagcacactaagtctcctattttggcttggaatcatgttcgacacgctcagttccgcaatgtaccagcgcccactagttgtctccgacgaagacagccagatcggctccgcctccccaacagcttcagccgaccatcgagtcaacctcaactactgggaaatcccagacaacgaccttccggcgaagaacgatgtctggggcgaattcttcctccaacccgcagcacgtcaagagccaacctccacacatcctcaactccaaccacaacaacctcgctggccctgctcttatgaagaagcggcctctgtcctctccgaagcgacaccggtcaaagtcctcctttaccgccgcatcactcaactccaaaccctcatctaccgtggctcttctccagctcgtcttgaagaagttatccaaaagaccctgcttgtgtaccaccactggacatgcacctatcaatcctttatgctcgactgtgtcgcaaaccacgaatccctgccgcatcgaattcaatcatggtatgttatcctcgacggccattggcacctggctgcgatgcttcttgccgatgtgctcgagtcaattgacagaagctacctcggtatggaatcggagcgggaatcccgaatcgcaagcgacctcatcgcaacacttcgcatcgacaacgcactcgcggtcggagcactagcccgcgcatcgctgcatggccagaatagcacgatgcatcgctactttcatgactcgttgaacgaggtcgcgttcctcgtcgaaccatggacggttgtgctaattcattcatttgcgaaggcggcgtatatttctctcgattgtttgggccagggacagggcggagcattagcagagtgtttccggcagaattgcgaatattgtatttgtgcgctgaagtatttggggaggaagtcggatatgcgtttgtt

SEQ ID NO: 78 - DNA, 2384 bp, Artificial Sequence
  PCR sequence generated using consensus oligonucleotides of A. navahoensis
gccctttgtgatccgtgtcggaaagggaagcgacgctgtgatgcaccggaaaataggaacgagaccaatgagaacggctgggcttcttgctcgaattgcaaacgttggaataaggattgtactttcaactggctgtcgtcgcagcgctccaagcctaagggggctgcaccccgggcgaggatgaagaaagccaggaccgctgcagccacggctgagccatcaaattcggctaccgcaatgcctacaccggaaagtggccatcaagatacacctcctattattaacgcctacgatgcgctaccgagctggagtcagggattggtctcccaccccggcgacctgtttgatttcagccaatcttctattcccatgcacacagatgatgcggtgaacgtgcagtcagaggtgcccttcccatgggatctggctattccgggcgacttcagcagcatgggccagcagctcgaaaaccccctcagtccgctcagttttcaagcagttattctcccgcctcacagtccgaacacggatgacctgatccacgagctggaagaacagtcaacggactctactaagtttgctggcctacggcgggatactcctgatgggtcgctgtggtctagtcgggcctcgccgctagcaccccagaacagcttgtgcattgcatcagacaaaacagcacagcaatatgctcgttcgtcgatgacaaagaatctgatgcgcatctatcatgacagcatggagaatgcactgtcttgctggctgacggagcacaactgcccctactccgaccagaccagctacctgccgcccaaacagagggcggaatggggtccgaactggtcgaacaggatgtgcatccgggtgtgccggctagaccgcgtatccacctcattacgcgggcgggccctgagcgcagaagaggacagagccgcagtccgagccctgaatctggccatcgtagcctttgcctcgcaatggacgcagcatgcgcagaagggagctgggctatctattcctacagacatagcaggcgatgagcgggccatccggagaaacacctggaacgaggcacgtcatgccttgcagcgctcgactgggatcccctcgttccgggtcatatttgcgaacatcatcttttctctgacacagagtgtgctggacgatagtgaacagcagggtgcgggtacacgtctagacaagttactcgagaatgaccgtgcgcctttgttcctggaaaccgccaatcgtcagctctgcacattccggcataagtttgcacggatgcaacgtcgaaggtcgactgccgaccagctccgaagggtatcagcagcatccgcgcttgcggatattttcgagacaccgacgccgtcgcctggaagcccccatctcgacccgattctagccaacgaggagcaccgcagtacactaagccttatgttctggctggggatcatgttcgacacactgagtgctgcaatgtaccagcgaccacttgtggtgtcagatgaggatagtcagatatcatcggcatccccgtcaacacagggttctgaaaccccaatcaacctagactgctgggagccaccaagacagattccaaacgatcgagctaaaagtgacgtatggggcgacctcttcctgcgcgactccgactccccccagcacgacaaatctcgcgcccagatctctcagccagcggctcgatggccctgcacctatgaacaagccgccgccgttctctcctccgcaacccccgtcaaagtcctcctctaccgccgtgtcacacagctccaaaccctcctctatcgcggcgccagtccggcccgcctggaagcagccatacagaaaacgatccatgtctaccaacactggacagaaaaataccagcccttcatgcaggactgcgtcgctaaccacgagctccttccctcgcgcatccagtcctggtacgtcatccttgacggccactggcacttagctgcgatgctgctagccgatgttctggagagcattgaccgcgacacgtactccgatatcgaccacaccgatctcgttacaaaactaagactcgataatgcgctggcagttagcgcccttgcgcgctcttcactcagagaccaggagcaatgtccagacaaagcatctcagatgtatcgccatttccacgactctttgaccgaagttgccttcctggtagagccgtggactgtcgtacttatccactcgtttgccaaggctgcgtatatcctcctggactgtttggatgtagacgggcagcgaagtaccctggctgggtatctgcagctgcagcagaattgcaattactgcattcgggcgctgcagtatttgggcaggaagtcggatatgcgtttgttaagggc

SEQ ID NO: 79 - DNA, 2369 bp, Artificial Sequence
  PCR sequence generated using consensus oligonucleotides of A. spectabilis
gccctttgtgatccgtgtcggaaagggaagagggggtgcgatgcgcctgaaaaccgaactgaaatcctcttcagttcctgctcgaactgcaaaaagtggaaaaaggagtgcacgttcaactggctgtccacaaatcccaccatcaaggccaagggaaaccaggaaaagaaaaggagaaaaactaaagcgaagccttgtactgtcgcggctgatacaagtacggatactgctactcctgatgatagtgtcggcatcccttcaattggcagtgatgttggcatcagcgtgggcgatggctcttatggtggctttatcgatgatggacttcagtctgcgcagtggttccctgtcaatccgggagacggtgatgtgttcgcgttgcccgggactgggttgttggacttgccttcgtcttcgttgttgttttcagaagcaggtatcgggggaaacgatacgagtgacccatatgcacagtctttagtctcgtggaacataggctttcctgacagttcgcaacttgacgctgtacctggaaagtctttcaccagacttgactctctacctacagactctctcgattacagattcgacgtgatccaacaactcgaagaagaattagcccaagattcgaggacattcccatccggcttctgcatggcctccgacaacacggccaaagcctacgctcgctcaacaatgacccacaaccttctccgcatatacaacgacggcatggagaacgcactatcatgctggctaacagagcataactgcccgtacaccgactcaatcggcgaccttctgctaccatacagccaaagaaaggaatggggcccggactggtcgaatcgcatgtgtatccgagtttgccacttagatcgcgcatcctctttgattcgcggtagggcgttgagcgcagaagaggacaagactgcagctcgagcgctgcatctagcgattgtggcatttgcctcgcagtggactcagcatgcgcaacggggaccggtcctatctgtccctgcgggaattgatgaagatgagaggttaattaagaaggatgtctggaatgaggcacgccatgcgctggagcactctacgaggattccctcgttccgggtgatctttgcgaatatcatcttctcgttgacgcagagtccgctagacaaaggcgacaggcgagatcaaggactgggtcagctactagagaacgacagcgcaccaatattcctcgagaacgccaacagacagctatacaccttccggcacaagttcaccaagctccagcgaagtaatcggaactcgccacaagtcgatcccatcctatctagtcaggaccaccgcagtacgctgaacctgctcttctggctcggaatcatgttcgacacgctaagtgcagcaatgtaccaacgccctctcgttgtctcagacgaggatagtcagatcacatcaatctcacctcctcccacaccggctccactcaactccccagcccaaatcaacctcgactgctgggacctcccctcagaccaaccacagaccacaacgctaacgttgcgccaaaagcaagacgtttggggcgacttcttcctccacccatcaccctccctctcacaccaagaacccaccacccagctcaaccctcaccctcagctagaacaccccaaacgctggccctgcacatacgccgaaccagcctcgatcctctcctctgcaacccccgtaaaagtcctcctctaccggcgcgtcacccaactccaaaacctcatctaccgcggtgcaacaccctcgcaactcgaattagtcatccagaagacactcctcgtctacaaccactggcagcaaacctacgcgcccttcatgacagactgcgtgaccaaccacgctattctcccgccgagaatccaatcctggtatgtcattttagacggccattggcatctcgctgcgatgttattggccgaagtagttgaggaaatcgataacgctaggctagggttagactctgcgcgagagacaagaaacatatcgaatttcgtcgagacgttaagaagggagaatgcattagccgttggcgcgctagccagggcgtcactgcagggtcagaatcccggtatggaagaacgttaccatgatagtgtgaatgaggttgcgtttctggtggagccgtggacggttgttctggtgaattgttttgcgaagggcgggtatatttcggctgagagggctgcgggttgttcgtcgtttactggggctggggttggagctggggatgggattggcgttggagaggtgtttcgtctgaattgtggattctgtatttgtgcgttggagtatcttggtaggaagtcggatatgcgtttgttaagggc

SEQ ID NO: 80 - DNA, 26 bp, Artificial Sequence
  Oligonucleotide Sally Three
gtcgacgaattcgcccttctcgaatg

SEQ ID NO: 81 - DNA, 26 bp, Artificial Sequence
  Oligonucleotide Sally Four
gtcgacgaattcgcccttttacacaa

SEQ ID NO: 82 - DNA, 20 bp, Artificial Sequence
  Oligonucleotide Sally14
ccattgcccagctatctgtc

SEQ ID NO: 83 - DNA, 18 bp, Artificial Sequence
  Oligonucleotide Alcust seq10r
tgcgcgtcattgtcgatc

SEQ ID NO: 84 - DNA, 21 bp, Artificial Sequence
  Oligonucleotide NPT2-2
tcgccttctatcgccttcttg

SEQ ID NO: 85 - DNA, 24 bp, Artificial Sequence
  Oligonucleotide p35S-3
ctcgccgtaaagactggcgaacag

SEQ ID NO: 86 - DNA, 22 bp, Artificial Sequence
  Oligonucleotide Sally 21
gtcgacgaattcgcccttatgg

SEQ ID NO: 87 - DNA, 24 bp, Artificial Sequence
  Oligonucleotide Sally 22
gtcgacgaattcgcccttaactac

SEQ ID NO: 88 - DNA, 18 bp, Artificial Sequence
  Oligonucleotide Alcfum seq4r
gacaagctctgctggtag

SEQ ID NO: 89 - DNA, 26 bp, Artificial Sequence
  Oligonucleotide Sally 12
gtcgacgaattcgcccttttcatggc

SEQ ID NO: 90 - DNA, 26 bp, Artificial Sequence
  Oligonucleotide Sally 13
gtcgacgaattcgcccttggttgctc

SEQ ID NO: 91 - DNA, 16 bp, Artificial Sequence
  Oligonucleotide M13 for
gtaaaacgacggccag

SEQ ID NO: 92 - DNA, 17 bp, Artificial Sequence
  Oligonucleotide M13rev
caggaaacagctatgac

SEQ ID NO: 93 - DNA, 18 bp, Artificial Sequence
  Oligonucleotide Alcvers seq2
cgccttagagcactcaac

SEQ ID NO: 94 - DNA, 18 bp, Artificial Sequence
  Oligonucleotide Alcvers seq1r
agtctgtggcttgtcgag

SEQ ID NO: 95 - DNA, 19 bp, Artificial Sequence
  Oligonucleotide Alcvers seq5r
ggaagcgaacatatcattg

SEQ ID NO: 96 - DNA, 28 bp, Artificial Sequence
  Oligonucleotide Knpflav for
ggtaccgaattcgcccttatgtcttatc

SEQ ID NO: 97 - DNA, 28 bp, Artificial Sequence
  Oligonucleotide flavkpnI rev-2
ggtacctcaaagggcgcacatatgatag

SEQ ID NO: 98 - DNA, 18 bp, Artificial Sequence
  Oligonucleotide Alcflav seq8r
ccattgagagtcatgtcg

SEQ ID NO: 99 - DNA, 30 bp, Artificial Sequence
  Oligonucleotide Sally 17P
cagggtacccgggggtcgaccgggctgcag

SEQ ID NO: 100 - DNA, 30 bp, Artificial Sequence
  Oligonucleotide Sally 18P
ctgcagcccggtcgacccccgggtaccctg

SEQ ID NO: 101 - DNA, 2526 bp, Aspergillus nidulans var. acristatus
atggcagatacgsgccgacgccagaatcatagctgygayccctgtcgcaagggcaagcggcgctgtgaygccccggtaggttgccgatatcggctccccagcgtctscactgatagtcactgagacgtaacacaggaaaatagaaatgaggccaatgaaaatggctgggtttcgtgctcaaattgcaagcgttggaacaaggattgtaccttcaattggctctcatcccaacgctccaaggcaaaaggggctgcacccagggcgagaacgaagaaagccaggactgcaacaaccaccagtgaaccatcaacttcagctgcaacaatccctacaccggaaagtgacaatcacgatgcgcctccagtcatcaacgctcacgacgcgctcccgagctggactcaggggctgctctcccaccccggcgaccttttcgatttcagccactctgctattcctgcgaatgcagaagatgcagccaacgtgcagtcagacgcaccttttccgtgggatctagctattcccggtgatttcagcatgggccaacagctcgagaaaccactcagtccgctcagttttcaaacagtccttttcccgccccatagcccgaacacggatgacctcattcgcgagctggaagagcagactacggatccggactcggttaccgatactaagagtgtgcaacaggtcgctcaagatggttcgatatggtctgatcggcagtcgccgctactgcctgagaacagtctgtgcatggcctcagacagcacagcacggcgatatgcgcgttcctcaatgacgaagaatctgatgcgcatctaccacgatagtatggagaatgcactatcctgctggctgacagagcacaattgtccatactccgaccaaatcagctacctgccgcccaagcagagggcggaatggggcccgaactggtcaaacaggatgtgcatccgggtgtgccggttagatcgcgtatctacctcactacgcgggcgcgccttgagtgccgaagaggacagagccgcagcccgagccctgcatctggcgatcgtagcttttgcgtcgcaatggacgcagcatgcgcagaggggggctgggttatctgttcctgcagacatagcggccgatgagagggccatcaggaggaacgcctggaatgaagcacgccatgccttgcagcacacgacggggattccgtcatttcgggttatatttgcgaatatcatcttttctctcacgcagagtgtgctggatgataatgagcagcagggtgtgggtgcacgtctggacaagctactcgaaaatgacggtgcgcccgtgttcctggaaactgcgaaccgtcmgctttatacattccgrcataagtttgcacgaatgcaacgccgcggtaaggctttcaacaggctcccggggggatctgtcgcatcgacattcgccggtattttcgagacaccgacgccgtcgtctgaaagcccacagyttgacccggttgtggccagtgaggagcatcgcagtacattaagccttatgttctggcttgggawcatgttcgatacactaagcgctgcaatgtaccagcgaccactcgtggtgtcagacgaggatagccagatatcatcggcatctccatcaacgcgcggctctgaaacgccgatcaacctagactgctgggaacccccaagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgccggtcaaagtcctcctctaccgccgcgtcacgcagctccaaacccttctctatcgcggcgccagccctgcccgccttgaagcggccatccagagaacgctccatgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtcacaaagctaaggctcgataatgcattagcagttagtgcccttgcgcgctcttcactccgaggccaggagctagacccgggcaaagcatctccgatgtatcgccatttccatgattctctgaccgaggtggcattcctggtagaaccgtggaccgtcgtccttattcactcgtttgccaaggctgcgtatatcttgctggactgtttagatctggacggccaaggaaatgcactagcggggtacctgcagctgcgacaaaattgcaactactgcattcgggcgctgcagtttctgggcaggaagtcggatatggcggcgctggttgcgaaggatttagagacaggtttgaatgggaaagttgacagctttttgtag

SEQ ID NO: 102 - DNA, 2532 bp, Aspergillus nidulans var. dentatus
  misc_feature (1844)..(1844): n= a, c, g, or t
atggcatgatacgcgccgacgccagaatcatagctgcgayccctgtcgcaagggcaagcgacgctgtgatgccccggtaggttgccgatatcggctccccagcgtgtgcactgacagtcgctgagatgtaacacaggaaaatagaaacgaggccaatgaaaacggctgggtttcgtgttcaaattgcaagcgttggaacaaggattgtaccttcaactggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagrcgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcgggtaccgatactaatagtgtacaacaggtcgcttcaaaacggatcgctatggtctgatcggcagtccccgctactgcctgagaacagtctgtgcatggcctcagacaagcacagcacggcgatatgcccgttccccaatgacgaagaatctgatgcgaatctaccccgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctctcgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctyttcctccgcacctyggactctctcccagatcacgaatcccacacacaaatctctcagccagcggctcgatggccctgcacctacgaacaggccgccgccgctctctcctntgcaacgcccgttaaagtcctcctctaccgccgcgtsacgcagctycaaacccctcctctatcgcggcgccagccctgcccgccttgaagcggccatcccagagaacgctctacgttttataatcactggacagcgaagtaccaaccatttatgcaggactgygttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcrcgctcttcactccgaggccaggagctggacccgggcaaagcatctccgatgtatcgccatttccatgattctctgaccgaggtggcattcctagtagaaccgtggaccgtcgttcttattcactcgtttgccaaagctgcgtatatctygctggactgtttagatctggacggccaaggaaatgcactagcggggtacctgcagctgcggcaaarttgcaactactgcattcgagcgctgcaatttctgggcaggaagtcggatatgsskkygytggttgcgaaggatttagagagaggtttgaatgggaaagttgacagctttttgtag

SEQ ID NO: 103 - DNA, 2449 bp, Aspergillus nidulans var. vuimellin
ccctttgtgatccgtgtcggaaggggaagcgacgctgtgatgccccggtaggttgccgatatcggctccccagcgtgtgcactgacagtcgctgagatgtaacacaggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatgcgtttgttaagggc

SEQ ID NO: 104 - protein, 11 aa, Artificial Sequence
  Consensus amino acid motif 1
CDPCRKGKXCD

SEQ ID NO: 105 - protein, 13 aa, Artificial Sequence
  Consensus amino acid motif 2
CXNCKXWXKXCXF

SEQ ID NO: 106 - protein, 14 aa, Artificial Sequence
  Consensus amino acid motif 3
NALSCWLTEHNCPY

SEQ ID NO: 107 - protein, 15 aa, Artificial Sequence
  Consensus amino acid motif 4
WSNMRCIXRVCXLDR

SEQ ID NO: 108 - protein, 10 aa, Artificial Sequence
  Consensus amino acid motif 5
RXRALSXXED

SEQ ID NO: 109 - protein, 10 aa, Artificial Sequence
  Consensus amino acid motif 6
FASQWTQHAQ

SEQ ID NO: 110 - protein, 14 aa, Artificial Sequence
  Consensus amino acid motif 7
RHAXXXXTXIPSFR

SEQ ID NO: 111 - protein, 11 aa, Artificial Sequence
  Consensus amino acid motif 8
FANIIFSLTQS

SEQ ID NO: 112 - protein, 16 aa, Artificial Sequence
  Consensus amino acid motif 9
FLEXXNRXXXXFRHKF

SEQ ID NO: 113 - protein, 6 aa, Artificial Sequence
  Consensus amino acid motif 10
MFDTLS

SEQ ID NO: 114 - protein, 16 aa, Artificial Sequence
  Consensus amino acid motif 11
AMYQRPLVVSDEDSQI

SEQ ID NO: 115 - protein, 8 aa, Artificial Sequence
  Consensus amino acid motif 12
DVWGXXFL

SEQ ID NO: 116 - protein, 11 aa, Artificial Sequence
  Consensus amino acid motif 13
ATPVKVLLYRR

SEQ ID NO: 117 - protein, 7 aa, Artificial Sequence
  Consensus amino acid motif 14
LDGHWHL

SEQ ID NO: 118 - protein, 10 aa, Artificial Sequence
  Consensus amino acid motif 15
NALAVXALAR

SEQ ID NO: 119 - protein, 13 aa, Artificial Sequence
  Consensus amino acid motif 16
EVAFXVEPWXXVL

SEQ ID NO: 120 - protein, 7 aa, Artificial Sequence
  Consensus amino acid motif 17
LXRKSDM

SEQ ID NO: 121 - DNA, 2463 bp, Aspergillus nidulans
atggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttg

SEQ ID NO: 122 - protein, 821 aa, Aspergillus nidulans
MADTRRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSDAPFPWDLAIPGDFSMGQQLEKPLSPLSFQAVLLPPHSPNTDDLIRELEEQTTDPDSVTDTNSVQQVAQDGSLWSDRQSPLLPENSLCMASDSTARRYARSTMTKNLMRIYHDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLPGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLYVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMAALVAKDLERGLNGKVDSFL

SEQ ID NO: 123 - protein, 233 aa, Artificial Sequence
  Consensus amino acid sequence of the AlcR orthologues
CXASDXTAXXXXRXXMXXNLXRIYXXXMXNALSCWLTEHNCPYXDXLXXXXRXEWGPXWSNRMCIXVCXLDRXSXSXRXRALSXXEDXFASQWTQHAQXGXXLXXPXXIXXXERXXXXXXWXXXRHAXXXXTXIPSFRXFANIIFSLTQSXXDXLLXXXXAPXFLEXXNRXXXXFRHKFXXXQRXSPXXDPXXXXXXHRXTLXLXFWXGXMFDTLSXAMYQRPLVVSDEDSQI

SEQ ID NO: 124 - protein, 834 aa, Aspergillus nidulans var. dendatus
  MISC_FEATURE (144)..(816): Xaa at positions 144, 562, 594, 775, 797, 815 and 816 is unknown
MADTRRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSXAPFPWDLAIPGDFSMGQQLEKPLSPLSFQAVLLPPHSPNTDDLIRELEEQTTDPDSGTDTNSVQQVAQNGSLWSDRQSPLLPENSLCMASDSTARRYARSPMTKNLMRIYPDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLSGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTXDSLPDHESHTQISQPAARWPCTYEQAAAALSXATPVKVLLYRRVTQLQTLLYRGASPARLEAAIPENALLPPKQRAEWGPNTFYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYIXLDCLDLDGQGNALAGYLQLRQXCNYCIRALQFLGRKSDMXXLVAKDLERGLNGKVDSFL

SEQ ID NO: 125 - protein, 821 aa, Aspergillus nidulans var. acristatus
  MISC_FEATURE (5)..(496): Xaa at  positions  5, 429, 475 and 496 is unknown
MADTXRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINAHDALPSWTQGLLSHPGDLFDFSHSAIPANAEDAANVQSDAPFPWDLAIPGDFSMGQQLEKPLSPLSFQTVLFPPHSPNTDDLIRELEEQTTDPDSVTDTKSVQQVAQDGSIWSDRQSPLLPENSLCMASDSTARRYARSSMTKNLMRIYHDSMENALSCWLTEHNCPYSDQISYLPPKQRAEWGPNWSNRMCIRVCRLDRVSTSLRGRALSAEEDRAAARALHLAIVAFASQWTQHAQRGAGLSVPADIAADERAIRRNAWNEARHALQHTTGIPSFRVIFANIIFSLTQSVLDDNEQQGVGARLDKLLENDGAPVFLETANRXLYTFRHKFARMQRRGKAFNRLPGGSVASTFAGIFETPTPSSESPQXDPVVASEEHRSTLSLMFWLGXMFDTLSAAMYQRPLVVSDEDSQISSASPSTRGSETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLHVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMAALVAKDLETGLNGKVDSFL

SEQ ID NO: 126 - protein, 794 aa, Aspergillus nidulans var. vuimellin
CDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSDAPFPWDLAIPGDFSMGQQLEKPLSPLSFQAVLLPPHSPNTDDLIRELEEQTTDPDSVTDTNSVQQVAQDGSLWSDRQSPLLPENSLCMASDSTARRYARSTMTKNLMRIYHDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLPGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLYVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMRLLR

SEQ ID NO: 127 - protein, 793 aa, Aspergillus faveolatus
CDPCRKGKRRCDAPENRNEANENGWVSCSNCKRWNKDCTFNWLSSQRSKPKGAAPRARTKKSRTATTTSEPATSAAAIPTPESDNHDAPPVINAHDALPSWTQGLLSHPGDLFDFSHSAIPANAEDAANVQSDAPFPWDLAVPGDFSMVQQLEKPLSPLSFQAVLLPPHSPNTDDLIRELEEQTTDPDSVTDTNSLQQVAQDGSLWSDRQSPLLPENSLCMASDSTARRYARSSMTKNLMRIYHDSMENALSCWLTEHNCPYSDQISYLPPKQRAEWGPNWSNRMCIRVCRLDRVSTSLRGRALSAEEDRAAARALHLAIVAFASQWTQHAQRGAGLSVPADIAADERAIRRNAWNEARHALQHTTGIPSFRVIFANIIFSLTQSVMDDNEQQGVGARLDKLLENDGAPVFLETANRQLYTFRHKFTRMQRRGKAFNRLPGGSVASTFADIFETPTLSSESPQLDPVVASEEHRSTLSLMFWLGIMFDTLSAAMYQRPLVVSDEDSQISSASPSTRGSETPINLDCWEPPRQVPSNHENSDVWGDLFLRTSGSLQEHESHTQISQPAARWPCTYEQAAAALSSATPVKVLLYRRVTQLQTLLYRGASPARLEAAIQRTLHVYNHWTAKYQPFMQDCVANHELLPSRIQSWYVILDGHWHLAAMLLADVLESIDRDSYSDTNHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMRLL

SEQ ID NO: 128 - protein, 821 aa, Aspergillus corrugatus
MDDTRRRQNHSCDPCRKGKRRCDAPENRNEANENGWVSCSNCKRWNKDCTFNWLSSQRSKPKGAAPRARTKKARTATTTSEPSTSAAAIPTPESDNHDAPPVINAHDPLPSWTQGLLSHPGDLFDFSQSSIPANAEDAANVQSDAPFLWDLAIPGDFSIGQQLEKPLSPLSFQAVLLPPHSPNTDDLIRELEEQTTDPDSVTDTNSLQQVAQDGSRWSDRQSQLLPENSLCMASDSTARRYARTSMTKNLMRIYHDSMENALSCWLTEHNCPYSDQISYLPPKQRAEWGPNWSNRMCIRVCRLDRVSTSLRGRALSAEEDRAAARALHLAIVAFASQWTQHAQRGAGLSVPADIAGDERAIRRNAWNEARHALQHTTGIPSFRVIFANIIFSLTQSVLDDTEQQNVGARLDRLLENDGAPVFLETANRQLYTFRHKFARMQRRGKAFNRLPVESVASTFADTFETPTPPSESPQLDPVVASEEHRSTLSLMFWLGIMFDTLSAAMYQRPLVVSDEDSQISSAYPSTRGSETPINLDCWEPPRQAPSNQEKSDVWGDLFLRTSDSLQGHESHTQISQPAARWPCTYEQAAAALSSATPVKVLLYRRVTQLQTLLYRGASPARLEAAIQRTLHVYNHWTAKYQPFMQDCVANHELLPSRIQSWYVILDGHWHLAAMLLADVLESIDRDAYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCVRALQFLGRKSDMAALVAKDLERGLNGKVDSFL

SEQ ID NO: 129 - protein, 795 aa, Aspergillus cleistominutus
CDPCRKGKRRCDAPENRNEANENSWVSCSNCKRWNKDCTFNWLSSQRSKPKGAAPRARTKKARAATTTSEPSTSAAAFPTPESDNHDAPPVINAHDALPSWTQGLLSHPSDLFDFSQSSIPANVEDAAANVQSDAPFPWDLAIPGDFSMGQQLEKPLSPLSFQAVLLPPHSPNTDDLIRELEEQTTDPDSVTDTNSLQQAAQHGSLWSDRHSPLLPENSLCMASDSTARRYARSSMTKNLMRIYHDSMENALSCWLTEHNCPYSDQISYLPPKQRAEWGPNWSNRMCIRVCRLDRVSTSLRGRALSAEEDRAAARALHLAIVAFASQWTQHAQRGAELSVPADIAADERAIRRNAWNEARHALQHTTGIPSFRVIFANIIFSLTQSVLDDTEQQGVGARLDRLLENDGAPVFLETANRQLYTFRHKFARMQRRGKAFNRLPGGSVASTFADIFETPTPSSESPQLDPVVASEEHRSTLSLMFWLGIMFDTLSAAMYQRPLVVSDEDSQISSASPSTRGSETPINLDCWEPPRQVPSNQDKSDVWGDLFLRASDSLQDHESHTQISQPAARWPCTYEQAAAALSSATPVKVLLYRRVTQLQTLLYRGASPARLEAAIQRTLHVYNHWTAKYQPFMQDCVTNHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLNLDSQGNALAGYLQLRQNCHCCIRALQFLGRKSDMRLLR

SEQ ID NO: 130 - protein, 792 aa, Aspergillus navahoensis
CDPCRKGKRRCDAPENRNETNENGWASCSNCKRWNKDCTFNWLSSQRSKPKGAAPRARMKKARTAAATAEPSNSATAMPTPESGHQDTPPIINAYDALPSWSQGLVSHPGDLFDFSQSSIPMHTDDAVNVQSEVPFPWDLAIPGDFSSMGQQLENPLSPLSFQAVILPPHSPNTDDLIHELEEQSTDSTKFAGLRRDTPDGSLWSSRASPLAPQNSLCIASDKTAQQYARSSMTKNLMRIYHDSMENALSCWLTEHNCPYSDQTSYLPPKQRAEWGPNWSNRMCIRVCRLDRVSTSLRGRALSAEEDRAAVRALNLAIVAFASQWTQHAQKGAGLSIPTDIAGDERAIRRNTWNEARHALQRSTGIPSFRVIFANIIFSLTQSVLDDSEQQGAGTRLDKLLENDRAPLFLETANRQLCTFRHKFARMQRRRSTADQLRRVSAASALADIFETPTPSPGSPHLDPILANEEHRSTLSLMFWLGIMFDTLSAAMYQRPLVVSDEDSQISSASPSTQGSETPINLDCWEPPRQIPNDRAKSDVWGDLFLRDSDSPQHDKSRAQISQPAARWPCTYEQAAAVLSSATPVKVLLYRRVTQLQTLLYRGASPARLEAAIQKTIHVYQHWTEKYQPFMQDCVANHELLPSRIQSWYVILDGHWHLAAMLLADVLESIDRDTYSDIDHTDLVTKLRLDNALAVSALARSSLRDQEQCPDKASQMYRHFHDSLTEVAFLVEPWTVVLIHSFAKAAYILLDCLDVDGQRSTLAGYLQLQQNCNYCIRALQYLGRKSDMRLLR

SEQ ID NO: 131 - protein, 795 aa, Aspergillus heterothallicus
CDPCRKGKRGCDAPELVGVQTFLTMIQEIRSGDGYTCSNCKRWKKKCTFNFVSSRRADARSVAANSRAKAKPTSTPVVATTASVATSVVAPPTPDSGNIPAMLNMGINTSEYNALLDEGLRSSQLDPARFGDMFEFMSPSNFAAEVLHAQSAIGGVNETLAWTMGVPGSWPMGMMPQSETSLSSLQSQELFISNEDANPYDVIQQLEDDFEDPATSVSKRDEDVRKFQWELCIASDKTANKVGRSTMNGNLIRIYHDSMENALSCWLTEHNCPYADPMSAMLPFNQRKEWGPSWSNRMCIRVCRLDRASSSIRGRALSVEEDRTAARALHLAIVAFASQWTQHAQKGTGLSVPAGIAYDERSTRKNIWNEARHALQHSTGIPSFRVVFANIIFSLTQSPLDETRPAKLAQLLDNDGAPVFLENANRQLYTFRHKFARLQREAPPPAATDLRRGSISSTLTEVLEIPTPESPQLDPILASQDHRSTLSLLFWLGIMFDTLSSAMYQRPLVVSDEDSQIGSASPTASADHRVNLNYWEIPDNDLPAKNDVWGEFFLQPAARQEPTSTHPQLQPQQPRWPCSYEEAASVLSEATPVKVLLYRRITQLQTLIYRGSSPARLEEVIQKTLLVYHHWTCTYQSFMLDCVANHESLPHRIQSWYVILDGHWHLAAMLLADVLESIDRSYLGMESERESRIASDLIATLRIDNALAVGALARASLHGQNSTMHRYFHDSLNEVAFLVEPWTVVLIHSFAKAAYISLDCLGQGQGGALAECFRQNCEYCICALKYLGRKSDMRL

SEQ ID NO: 132 - protein, 786 aa, Aspergillus spectabilis
CDPCRKGKRGCDAPENRTEILFSSCSNCKKWKKECTFNWLSTNPTIKAKGNQEKKRRKTKAKPCTVAADTSTDTATPDDSVGIPSIGSDVGISVGDGSYGGFIDDGLQSAQWFPVNPGDGDVFALPGTGLLDLPSSSLLFSEAGIGGNDTSDPYAQSLVSWNIGFPDSSQLDAVPGKSFTRLDSLPTDSLDYRFDVIQQLEEELAQDSRTFPSGFCMASDNTAKAYARSTMTHNLLRIYNDGMENALSCWLTEHNCPYTDSIGDLLLPYSQRKEWGPDWSNRMCIRVCHLDRASSLIRGRALSAEEDKTAARALHLAIVAFASQWTQHAQRGPVLSVPAGIDEDERLIKKDVWNEARHALEHSTRIPSFRVIFANIIFSLTQSPLDKGDRRDQGLGQLLENDSAPIFLENANRQLYTFRHKFTKLQRSNRNSPQVDPILSSQDHRSTLNLLFWLGIMFDTLSAAMYQRPLVVSDEDSQITSISPPPTPAPLNSPAQINLDCWDLPSDQPQTTTLTLRQKQDVWGDFFLHPSPSLSHQEPTTQLNPHPQLEHPKRWPCTYAEPASILSSATPVKVLLYRRVTQLQNLIYRGATPSQLELVIQKTLLVYNHWQQTYAPFMTDCVTNHAILPPRIQSWYVILDGHWHLAAMLLAEVVEEIDNARLGLDSARETRNISNFVETLRRENALAVGALARASLQGQNPGMEERYHDSVNEVAFLVEPWTVVLVNCFAKGGYISAERAAGCSSFTGAGVGAGDGIGVGEVFRLNCGFCICALEYLGRKSDMRLL

SEQ ID NO: 133 - protein, 780 aa, Aspergillus bicolor
  MISC_FEATURE (13)..(14): Xaa at positions 13 and 14 is unknown
CDPCRKGKRGCDXXENRTEILFNSCSNCKKWKKECAFNWLATNPTIKGKGNQEKNRRTKAKPSTAATDTNTAIATPDDSVDIPSVGSDVGISVGDGSYGSCIDDGLQSAQWFPVNPGNGDVLALPGTGLFDLTSSSLLFPEGGIGGNDTSDPYAQSIISWNMGGFPDNWQLGAVPGKSFARLDLPTNSLDDTFDIIQPLEEDSSRNSRTFPSGFCIASDNTAKAYARSTMTRNLLRIYHGSMDNALSCWLTEHNCPYIDSIGDLLLLYSQRKEWGPNWSNRMCIVCQLDRASSSIRSRALSAEEDMTMVFASQWTQHAQRGPVLSVPAGIDENERSIRKNVWDEIRHAQEHSTRIPSFRVIYAFANIIFSLTQSPLDKGERRGDGLGQLLENYSAPIFLENTNRQRYPFRHKFTRLQRRNRSSPQVDPILSSQDHRGTLNLLFWFGIMFDTLSAAMYQRPLVVSDEDSQIASISPPPPTPSPLNPPAQNNLECWNFPSDQPQTTTLTIRQKQDVWGYSFLHPTASLSHQEPTTQLNPHPQPKHRPKRWPCTYAESASILSFATPVKVLLYRRVTQLQTLIYRGAAPSQLESVIQKTLLVYNHWQQFYAPFMTDYVTNHAILPPRIHSWCVMLDGHWHLAAMLLAVVVEETDNAGLGLDSAREARNLSDFVGTLRRENALAVGALARAPLQGQNPGMEEHYHNSLNEVAFPVEPWAAVLVYCFAKGGGGLYIPLERVGYSSFTRDGSGDGVKDGKVFRLNCELCICVSEYLGRKSDMRLGG

SEQ ID NO: 134 - DNA, 20 bp, Artificial Sequence
  Oligonucleotide Alcvers seq4r
caaattgtgcgtcatcgttg

SEQ ID NO: 135 - DNA, 19 bp, Artificial Sequence
  Oligonucleotide Alcvers seq 5r
ggaagcgaacatatcattg

SEQ ID NO: 136 - DNA, 20 bp, Artificial Sequence
  Oligonucleotide Alcvers for
ggttgctcgccatggatgac

SEQ ID NO: 137 - DNA, 22 bp, Artificial Sequence
  Oligonucleotide Alcust for
ctcgaatgaagatgggagactc

SEQ ID NO: 138 - DNA, 22 bp, Artificial Sequence
  Oligonucleotide Alcust rev
ttacacaaggatatccgctgac

SEQ ID NO: 139 - DNA, 19 bp, Artificial Sequence
  Oligonucleotide Alcflav seq6r
gaagatcgaaagtgtgatg

SEQ ID NO: 140 - DNA, 21 bp, Artificial Sequence
  Oligonucleotide Alcflav for
atgtcttatcgtcgccgtcag

SEQ ID NO: 141 - DNA, 18 bp, Artificial Sequence
  Oligonucleotide Alcflav seq 7r
actctccacactcgtgag

SEQ ID NO: 142 - DNA, 18 bp, Artificial Sequence
  Oligonucleotide Alcflav seq 8r
ccattgagagtcatgtcg

SEQ ID NO: 143 - DNA, 24 bp, Artificial Sequence
  Oligonucleotide Alcfum for
atggaggctcatcgtcgacgccag

SEQ ID NO: 144 - DNA, 20 bp, Artificial Sequence
  Oligonucleotide Alcfum RT
caaagccaggtggcgaagag

SEQ ID NO: 145 - DNA, 19 bp, Artificial Sequence
  Oligonucleotide ITS
tccgtaggtgaacctgcgg

SEQ ID NO: 146 - DNA, 19 bp, Artificial Sequence
  Oligonucleotide ITS
tcctccgcttattgatatg

SEQ ID NO: 147 - DNA, 420 bp, Aspergillus nidulans
taagtcccttcgtatttctccgcctgtgtggagctaccatccaataacccccagctgaaaaagctgattgtcgatagttgtgatagttcccacttgtccgtccgcatcggcatccgcagctccggatagttccgacctaggattggatgcatgcggaaccgcacgagggcggggcggaaattgacacaccactcctctccacgcagccgttcaagaggtacgcgtatagagccgtatagagcagagacggagcactttctggtactgtccgcacgggatgtccgcacggagagccacaaacgagcggggccccgtacgtgctctcctaccccaggatcgcatcctcgcatagctgaacatctatataaagacccccaaggttctcagtctcaccaacatcatcaaccaacaatcaacagt

(19) United States
(12) Patent Application Publication
(10) Pub. No.: US 2004/0157287 A1
(43) Pub. Date: Aug. 12, 2004

(54) Regulatory elements suitable for use in gene expression

(76) Inventors: Andrew James Greenland, Berkshire (GB); Ian Jepson, Berkshire (GB); Alberto Martinez, Berkshire (GB); Lynne Marie Roxbee Cox, Berkshire (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/468,250

(22) PCT Filed: Feb. 13, 2002
(86) PCT No.: PCT/GB02/00633

(30) Foreign Application Priority Data
Feb. 13, 2001 (GB) 0103524.5
Feb. 13, 2001 (GB) 0103523.7
Feb. 13, 2001 (GB) 0103521.1
Feb. 13, 2001 (GB) 0103520.3

(51) Int. Cl. C07K 14/47; C07H 21/04; C12N 15/85

(52) U.S. Cl. 435/69.1; 435/455; 435/320.1; 435/325; 530/350; 536/23.5

(57) ABSTRACT

The present invention relates to novel AlcR regulatory elements and nucleic acid sequences coding therefor, and their use in controlling gene expression in organisms such as plants. DNA constructs containing such nucleic acids, in particular, expression cassettes comprising inducible promoter elements and regulatory elements of the invention, which are capable of acting as “gene switches”, form further aspects of the invention.

The present invention relates to regulatory elements and nucleic acid sequences coding therefore, and their use in controlling gene expression in organisms such as plants. The invention also relates to DNA constructs containing such nucleic acids and to organisms incorporating such constructs. In particular, the invention relates to expression cassettes comprising inducible promoter and regulatory elements capable of acting as “gene switches” (GENESWITCH is a trademark owned by a Syngenta Group Company).

Recombinant DNA technology encompasses the manipulation of a wide variety of organisms for a huge range of purposes. Expression of genes that are foreign to a host organism, or alteration in the expression patterns of endogenous genes provides a means of modifying and/or improving the properties of the organism.

Expression of such genes is controlled by regulatory elements and when transforming an organism with such a gene it is important to ensure that suitable regulatory elements are included and arranged so that the gene is expressed in the desired manner. For example, it may be required that the gene is expressed only for a limited period, or only in a specific cell or tissue, in order to achieve the desired modification to the organism.

Gene switches provide a very useful addition to the “armoury” of the biotechnologist. The expression “gene switch” as used herein refers to a control sequence and regulator of such sequence that are responsive to a chemical inducer, which is applied exogenously. Applying a chemical inducer to, or withdrawing the chemical inducer from, an organism comprising a gene under the control of such a gene switch thus regulates expression of that gene.

The transformation of plants, in particular crop plants, in order to improve characteristics such as productivity or quality (in the case of crops), or to control fertility (in particular in the production of hybrid plants) or to introduce resistance to herbicides or insecticides, is well known. In general, this requires the expression of one or more foreign or endogenous genes in one or more plant tissues. Frequently, transient expression of these genes is necessary and/or desirable and gene switches are particularly useful in regulating such transient expression.

An example of a gene switch that has been found to be particularly useful in the manipulation of plants is derived from the fungal organism Aspergillus nidulans. This organism expresses the enzyme alcohol dehydrogenase I (ADH1; encoded by the alcA gene) only when it is grown in the presence of various alcohols or ketones. The induction is relayed through a regulator protein encoded by the constitutively expressed alcR gene. In the presence of inducer (alcohol or ketone), the regulator protein activates the expression of ADH1. This means that high levels of the ADH 1 enzyme are produced under inducing conditions (i.e. when alcohol or ketone are present). Conversely, the alcA gene (and thus ADH 1) are not expressed in the absence of inducer. In summary, the alcA promoter is an inducible promoter, activated by the AlcR regulator protein in the presence of inducer (i.e. by the protein/alcohol or protein/ketone combination). Expression of alcA and production of the enzyme is also repressed in the presence of glucose.

Both an alcR and an alcA gene (including the respective promoters) have been cloned and sequenced from Aspergillus nidulans (Lockington R A et al., 1985, Gene, 33: 137-149; Felenbok B et al., 1988, Gene, 73: 385-396; Gwynne et al., 1987, Gene, 51: 205-216). The nucleotide sequence of this alcR gene corresponds to SEQ ID NO 122 and the polypeptide sequence encoded thereby corresponds to SEQ ID NO 121 as described herein.

Alcohol dehydrogenase (adh) genes have been investigated in certain plant species. In maize and other cereals they are switched on by anaerobic conditions. The promoter region of adh genes from maize contains a 300 bp regulatory element necessary for expression under anaerobic conditions. However, no equivalent to the AlcR regulator protein has been found in any plant. Thus the alcA/alcR type of gene regulatory system is not known in plants. Thus, constitutive expression of alcR in plant cells does not result in the activation of any endogenous adh activity. The alcA/alcR gene regulatory system is therefore a particularly useful gene switch for plant use, since it can be used to control expression of a gene of interest (e.g. a transgene) without interfering with or interrupting any other plant cell function.

WO 93/21334 describes the production of transgenic plants that include such a system as a gene switch. This document specifically describes a chemically inducible plant gene expression cassette comprising a first promoter operatively linked to a regulator sequence encoding a regulator protein, the AlcR protein of Aspergillus nidulans, and an inducible promoter such as the Aspergillus nidulans alcA promoter or a chimeric promoter including elements of the alcA promoter, operatively linked to a gene of interest. In the presence of an exogenous inducer, the regulator protein activates the inducible promoter, thus mediating activation of the gene of interest. Exogenous chemical inducers that may be applied include those described by Creaser et al, (1984), e.g butan-2-one (ethyl methyl ketone), cyclohexanone, acetone, butan-2-ol, 3-oxobutyric acid, propan-2-ol and ethanol. Esters, such as those described in WO00/44917 may also be used as an exogenous inducer.

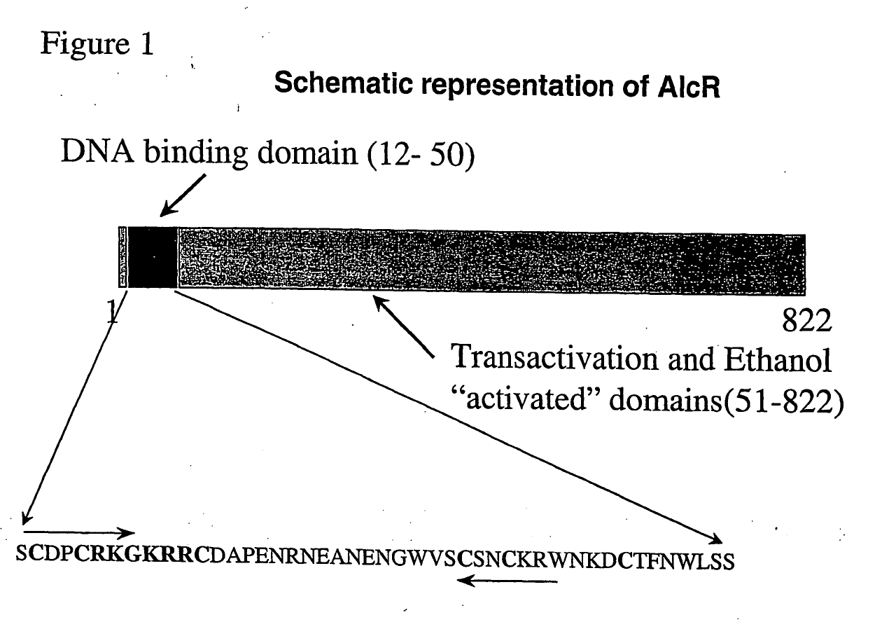

At present there is very little information available to provide clues as to where in AlcR the different functional domains are located. AlcR is known to be a transcription factor that auto-regulates its own promoter as well as regulating the activity of a cluster of genes including alcA (encoding alcohol dehydrogenase) and aldA (encoding aldehyde dehydrogenase). AlcR belongs to the Zn_{[2]}-Cys_{[6]} fungal type, binuclear family of transcription factors. In yeast this family comprises 58 polypeptides (e.g. Gal4, UGA3 and others). The AlcR protein is 780 amino acids long of which amino acids 12 to 50 comprise the DNA binding region, suggesting that 720 amino acids lying beyond the C-terminal end of the DNA binding domain remain uncharacterised (sec FIG. 1). An NMR structure of the AlcR DNA binding domain comprising the Zn_{[2]}-Cys_{[6]} has been reported by Cerdal et al. (1997, FEBS letter 408, 235-240). This indicates that in vitro AlcR can bind to DNA as a monomer. However, in vivo evidence indicates that the AlcR protein either binds as a homodimer or with the aid of another factor (Kulmburg, P et al., 1992, Molecular and Cellular Biology 12, 1932-1939; Panozzo, C., et al., 1997, Journal of Biological Chemistry 272, 22859-22865). A mutational study, where AlcR proteins were produced with a five amino acid deletion in, the N-terminal region and with a single amino acid alteration at Arg6, demonstrated that such mutated proteins were unable to bind to a monomeric DNA binding site in vitro, and in vivo (in Aspergillus) they were unable to induce alcA gene expression in the presence of ethanol. This inability of the mutated AlcR to induce alcA was shown to be a result of the mutated AlcR being unable to bind to its own promoter or those of other genes also under its control (Nikolaev, I., et al. (1999). Molecular Microbiology 31, 1115-1124).

Studies to date have concentrated on the AlcR DNA binding domain. Other functional domains such as those involved transactivation, ligand dependant transactivation, ligand binding, and nuclear localisation remain have not been structurally defined or characterised.

The applicants have identified and characterised a family of novel polypeptide orthologues of the A. nidulans AlcR regulator protein, through the isolation and characterisation of novel alcR polynucleotide sequences from different Aspergillus species. Characterisation of the family has identified a plurality of conserved amino acid motifs thought to be functionally and/or structurally important for AlcR regulator protein activity. These novel alcR orthologues provide useful alternatives to the A. nidulans alcR gene that is employed in the gene switch systems described above.

According to the present invention there is provided a polypeptide capable of activating an alc inducible promoter in the presence of a chemical inducer, provided that the polypeptide does not have the amino acid sequence specified in SEQ ID No 121.

The expression “alc inducible promoter” as used herein relates to any inducible promoter, which is part of the cluster of genes described above. These include, for example, alcA, aldA, aclB, alcR or alcC promoters obtainable from fungi. For example, an alc inducible promoter may be an alcR gene promoter obtainable from an Aspergillus species, such as an alcR promoter from A. nidulans, A. ustus, A. fumigatus, A. versicolor, A. flavus, A. faveolatus, A. corrugatus, A. cleistominutus, A. navahoensis, A. heterothallicus, A. spectabilis, or A. bicolor. Particularly suitable alcR promoters are those found in A. nidulans, A. ustus, A. flavus or A. versicolor, with specific examples being provided by SEQ ID NO 39, SEQ ID NO 60 and SEQ ID NO 47. Alternatively the alc inducible promoter may be the known alcA promoter from A. nidulans (SEQ ID NO 147).

As described above, the applicants have identified a number of amino acid motifs that are conserved throughout the novel AlcR polypeptide members identified herein. This high level of conservation is indicative of these motifs playing an important structural and/or functional role in the AlcR polypeptide, for example these motifs (either separately or in combination) may form part of the transactivation, ligand dependant transactivation, ligand binding, or nuclear localisation domains. These motifs may be thus be used to define (and thus identify further) members of the AlcR protein family. Any further novel AlcR orthologues identified in this way may also be used as a component of the gene switch systems described herein. Thus in one embodiment the invention provides a polypeptide comprising at least one of the following amino acid motifs: motif 1 CDPCRKGKXCD (SEQ ID NO 104); motif 2 CXNCKXWXKXCXF (SEQ ID NO 105); motif 3 NALSCWLTEHNCPY (SEQ ID NO 106); motif 4 WSNMRCI(X)₀₋₁RVCXLDR (SEQ ID NO 107); motif 5 RXRALS(X)₂ED (SEQ ID NO 108); motif 6 FASQWTQHAQ (SEQ ID NO 109); motif 7 RHA(X)₄TXPSFR (SEQ ID NO 110); motif 8 FANIIFSLTQS (SEQ ID NO 111); motif 9 FLE(X)₂NR(X)₄FRHKF (SEQ ID NO 112); motif 10 MFDTLS (SEQ ID NO 113); motif 11 AMYQRPLVVSDEDSQI (SEQ ID NO 114); motif 12 DVWG(X)₂FL (SEQ ID NO 115); motif 13 ATPVKVLLYRR (SEQ ID NO 116); motif 14 LDGHWHL (SEQ ID NO 117); motif 15 NALAVXALAR (SEQ ID NO 118); motif 16 EVAFXVEPW(X)₂VL (SEQ ID NO 119); or motif 17 LXRKSDM (SEQ ID NO 120). Preferably a polypeptide of the invention will comprise at least two of the above-mentioned amino acid motifs, wherein the second motif is not the same as the first motif. Even more preferably a polypeptide of the invention will comprise at least 3,4,5,6,7,8,9,10,11,12,13,14,15,16, or 17 of the above-mentioned motifs, wherein each motif is different. Most preferably a polypeptide of the invention will have the consensus sequence shown in SEQ ID NO 123. Specific examples of polypeptides of the invention are provided by SEQ ID NOs 124, 125, 126, 127, 128, 129, 130, 131, 132, 133, 59, 18, 62, 66.

For the avoidance of doubt, all amino acid sequences described herein use the standard single letter code, wherein “X” represents any amino acid and a sub-scripted number denotes the number of residues of the type described within the preceding brackets, for example, motif 16 described above reads EVAFXEPWXXVL (wherein X is any amino acid) when written in full.

In addition to the highly conserved amino acid motifs described above, analysis of the members of the AlcR polypeptide members identified herein has revealed that they possess a further striking structural feature: they comprise a surprisingly high number of doublet and triplet amino acid repeats.

The term doublet amino acid repeat as used herein means that repeats of two consecutive identical amino acids are found throughout a polypeptide sequence. For example, motif 13 (ATPVKVLLYRR) comprises two doublet amino acid repeats one is “LL” and the second is “RR: it can be seen that more than 36% of the motif is comprised by doublet repeats.

Similarly the term triplet amino acid repeat as used herein means that repeats of at least three consecutive identical amino acids are found throughout a polypeptide sequence. For example, the A. fumigatus AlcR othologue (SEQ ID NO 18) comprises a total of 6 triplet amino acid repeats: 1 triplet of alanine (i.e. 1× at least AAA), 1 triplet of arginine (i.e. 1× at least RRR) and 4 triplets of serine (i.e. 4× at least SSS).

When expressed as a percentage, at least 7.5% of a polypeptide of the invention is comprised of doublet repeats, and in the specific examples of polypeptides described herein this percentage may be 12% or higher. Similarly with respect to triplet repeats, at least 1% of a polypeptide of the invention is comprised of triplet repeats and in the specific examples of polypeptides described herein, the percentage may be 2.5% or greater. In general polypeptides of the invention comprise a significant number of doublet repeats of proline, glutamine, arginine, serine, leucine and/or threonine. In particular it can be seen that polypeptides of the invention comprise at least 6 doublet repeats of leucine, at least 3 doublet repeats of serine and at least 4 doublet repeats of threonine. Such polypeptides may also comprise at least 3 doublet repeats of alanine, at least 1 doublet repeat of cysteine, at least 1 doublet repeat of aspartic acid, and at least 1 doublet repeat of proline.

Thus in a further embodiment there is provided a polypeptide according to any one of those previously described embodiments, the amino acid sequence of which comprises a plurality of at least doublet repeats of amino acid residues, wherein the plurality of at least doublet repeats comprise greater than 7.5%. Preferably such a polypeptide comprises at least 6 doublet repeats of leucine, at least 3 doublet repeats of serine and at least 4 doublet repeats of threonine. Even more preferably, such a polypeptide will also comprise at least 3 doublet repeats of alanine, at least 1 doublet repeat of cysteine, at least 1 doublet repeat of aspartic acid, and at least 1 doublet repeat of proline.

In a further embodiment a polypeptide of the invention comprises a plurality of at least triplet repeats of amino acid residues, wherein the plurality of at least triplet repeats comprise greater than 1% of the polypeptide.

Where the terms “doublet repeat” and “triplet repeat” as used above are qualified by “at least”, it is meant that each repeat comprises at least two consecutive identical amino acid residues (i.e. it is at least a doublet) or at least three consecutive amino acid residues (i.e. it is at least a triplet), as appropriate. A second aspect of the invention provides nucleic acids encoding a polypeptide according to the first aspect of the invention described above. Specific examples of such nucleic acids are provided by SEQ ID NOs 102, 101, 103, 76, 74, 75, 78, 77, 38, 61, 46, 65, 79, 73, 58, and 17. However, the skilled man will appreciate that due to the degeneracy of the genetic code, a plurality of different nucleic acids may encode each polypeptide of the invention. It is intended that this aspect of the invention encompasses all such nucleic acids.

In a third aspect of the invention there is provided an expression cassette comprising: (i) a first promoter, (ii) a first nucleic acid encoding a polypeptide of the invention, wherein the polypeptide is capable of activating an alc inducible promoter in the presence of an exogenous chemical inducer, and wherein the first nucleic acid is under the control of the first promoter, (iii) a second promoter that is inducible by the polypeptide encoded by the first nucleic acid in the presence of the exogenous chemical inducer, and (iv) a second nucleic acid, the expression of which is under the control of the second promoter.

The first promoter of an expression cassette of the invention may be any promoter that is operative in the host organism. It may be a constitutive promoter, a tissue- or developmentally-specific promoter, or an inducible promoter. However, it is necessary that the polypeptide encoded by the first nucleic acid is expressed in a temporally and spatially desirable manner i.e. in the right cells of the host organism and at the right time in order to mediate the expression of the second nucleic acid as required. Thus in one embodiment, a tissue specific promoter, for example a flower specific promoter (such as an anther-specific or stigma-specific promoter) is employed. In another embodiment it is preferred that the promoter is a developmental-specific promoter. Particularly preferred tissue-specific and developmental-specific promoters are those which control gene expression during seed formation and germination, such as cysteine proteinsase promoters (as specified in International Publication No WO WO 97/35983) and the malate synthase promoter. In yet a further embodiment the first promoter will be a constitutive promoter. Where the host organism is a plant, examples of suitable constitutive promoters include, but are not limited to, the cauliflower mosaic virus 35S promoter, the ferrodoxin-RolD promoter, the maize ubiquitin promoter and the rice actin promoter.

The second promoter employed in an expression cassette of the invention may be any alcA, aldA, aclB, alcR or alcC promoter obtainable from fungi, in particular from Aspergillus species, examples of which include A. nidulans, A. ustus, A. fumigatus, A. versicolor, A. flavus, A. faveolatus, A. corrugatus, A. cleistominutus, A. navahoensis, A. heterothallicus, A. spectabilis, and A. bicolor. Alternatively, it may be a “chimeric” promoter sequence, created by fusing heterologous upstream and downstream regions as described in WO 93/21334. Typically in such chimeric promoters, the upstream region contains a promoter regulatory sequence and the downstream region contains a transcription initiation sequence, with the upstream and downstream regions being heterologous. Where such a chimeric promoter is employed as the second promoter in an expression cassette of the invention, it is preferred that the upstream region is derived from an inducible alcA, aldA, alcB, alcR or alcC promoter (as described above). The downstream sequence may be derived from the core promoter region of any promoter operative in the host organism into which the expression cassette is to be introduced. Thus where the host organism is a plant, it is preferred that the downstream promoter region is derived from a plant-operative promoter, such as the CaMV35S, ferrodoxin-RolD, maize ubiquitin and rice actin promoters. Alternatively the downstream promoter region may be synthesised from consensus promoter sequence.

However, it is preferred that the second promoter sequence is, or comprises parts of, a regulatory element of an alcA or an alcR promoter sequence obtainable from an Aspergillus species, such as A. nidulans, A. ustus, A. fumigatus, A. versicolor, A. flavus, A. faveolatus, A. corrugatus, A. cleistominutus, A. navahoensis, A. heterothallicus, A. spectabilis, and A. bicolor. Particularly suitable alcR promoters for use in this aspect of the invention are those found in A. nidulans, A. ustus, A. flavus and A. versicolor, with specific examples being provided by SEQ ID NO 39, SEQ ID NO 60 and SEQ ID NO 47. It is most preferable however, that the second promoter sequence is the known alcA promoter from A. nidulans (SEQ ID NO 147).

The alcR promoters of A. ustus, A. flavus and A. versicolor (SEQ ID NO 39, SEQ ID NO 60 and SEQ ID NO 47) disclosed herein are novel and form yet a further aspect of the invention.

Thus the invention further provides an alcR promoter sequence obtainable from A. ustus, A. flavus or A. versicolor, or a modified form or fragment thereof, which acts as an inducible promoter in the presence of an AlcR regulator protein (in particular an AlcR regulator protein of the invention), and an exogenous chemical inducer.

The expression “modified form” relates to a promoter that shares identity with an alcR promoter sequence obtainable from A. ustus, A. flavus or A. versicolor, but may include a number of differences in the nucleotide sequence which do not significantly affect the promoter activity. Preferably these differences are such that the overall identity between the two sequences is greater than 70%, more preferably greater than 80% and most preferably greater than 90%, when compared using, for example, the WILBUR-Lipman method with parameters set as follows: ktuple=3, gap penalty=3 and window=20.

The term “parts” used in relation to the promoters of the invention refers to truncated forms or active regions of the promoter, which retain promoter activity and which may be useful when combined with other promoter elements to form chimeric promoters as discussed above. Particularly preferred regions in the context of the present invention, are those promoter regulatory sequences that may be used in combination with heterologous transcription initiation sequences in chimeric promoters as outlined above.

The second nucleic acid of an expression cassette of the invention may be any nucleic acid that it is desired to be expressed in a host organism. Where the host organism is a plant, the second nucleic acid may encode all or part of either an endogenous plant protein or a foreign protein. Furthermore, the second nucleic acid may act as a sense or antisense nucleic acid that is required to be expressed in a controlled manner in order to modify the properties of the plant. The second nucleic acid may comprise a single gene or a series of genes.

Gene expression cassettes of the invention may be on the same construct, or may be divided into two parts. Where the expression cassette comprises two parts, one part will comprise elements (i) and (ii) subcloned into an appropriate expression vector, such as a plant expression vector. The second part will comprise at least part of the second promoter arranged to control expression of a downstream nucleic acid.

Expression cassettes of the invention may be transformed or transfected into the cell(s) of any suitable host organism. Suitable host organisms include microorganisms, (such as bacteria and yeasts) as well as plants and animals. However, it is preferred that the host organism is a plant.

In practice the construct(s) comprising an expression cassette of the invention are inserted into a host cell, such as a plant cell, by transformation. Where the host is a plant, the expression cassette will be a plant gene expression cassette and any transformation method suitable for the plant or plant cells may be employed. Such methods include infection with Agrobacterium tumefaciens containing recombinant Ti plasmids, electroporation, microinjection of cells and protoplasts, microprojectile transformation and pollen tube transformation. Where desired, whole plants having the new nucleic acid stably incorporated into the genome may be regenerated from such transformed cells. Both monocotyledonous and dicotyledonous transgenic plants may be obtained in this way.

Examples of transgenic plants which may be thus produced include field crops, cereals, fruit and vegetables such as: canola, sunflower, tobacco, sugarbeet, cotton, soya, maize, wheat, barley, rice, sorghum, tomatoes, mangoes, peaches, apples, pears, strawberries, bananas, melons, potatoes, carrot, lettuce, cabbage, and onion.

In a further aspect, the invention provides a cell, in particular a plant cell comprising an expression cassette of the invention. The expression cassette may be stably incorporated in the genome of the host cell by transformation. Yet further aspects of the invention provides a plant tissue or a plant comprising such cells, as well as progeny plants or seeds derived therefrom.

It is preferred that plant cells, tissue and/or plants according to the above-mentioned aspects of the invention are plant cells, tissue and/or plants of canola, sunflower, tobacco, sugarbeet, cotton, soya, maize, wheat, barley, rice, sorghum, tomato, mango, peach, apple, pear, strawberry, banana, melon, potato, carrot, lettuce, cabbage, or onion, and in particular cotton, soya, maize, wheat, barley, rice or sorghum.

As described above, expression cassettes of the invention may used to regulate gene expression in the host organism into which they are introduced. This is achieved through the exogenous application (or withdrawal) of a suitable chemical inducer. In the presence of a suitable exogenous chemical inducer, the regulator protein produced by the cassette will activate the expression of the second nucleic acid by stimulating the second inducible promoter also present in the cassette. Thus expression of the second nucleic acid may be regulated by external application of an inducer to the host. Thus in a further aspect of the invention there is provided method for controlling gene expression in a cell, comprising transforming a cell with an expression cassette of the invention, and applying an exogenous chemical inducer to the cell in order to induce transcription of the second nucleic acid.

The inducer may be any effective chemical (such as an alcohol or ketone). Suitable chemicals for use with an alcA/alcR-derived cassette include those listed by Creaser et al (1984, Biochem J, 225, 449454) e.g. butan-2-one (ethyl methyl ketone), cylcohexanone, acetone, butan-2-ol, 3-oxobutyric acid, propan-2-ol, and ethanol. Other suitable inducers include agriculturally acceptable esters, such as those described in WO0/44917. Such agriculturally acceptable esters generally comprise a compound of formula (I)

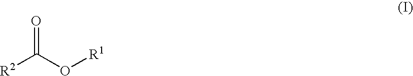

in which R¹ is a lower alkyl, lower alkenyl or lower alkynyl group, and R² is a organic group such that R²COOH is an agriculturally acceptable acid. Hydrolysis of a compound of formula (I) yields an alcohol of formula (II)

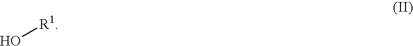

The term “agriculturally acceptable” as used herein means that the compounds may be applied to a particular soil or crop situation without causing unacceptable levels of soil damage or phytotoxicity in the crop. The expression “lower alkyl” as used herein includes C₁₋₆ alkyl groups, preferably from C₁₋₄ alkyl groups which may be straight or branched chain. Similarly the terms “lower alkenyl” and “lower alkynyl refer to groups which may have from 2-6 and preferably from 24 carbon atoms in a straight or branched chain.

The efficacy of plant gene expression cassettes of the invention may be demonstrated by transforming plant protoplasts either separately or together with suitable regulator and reporter constructs and conducting transient gene expression assays.

For example, it would be expected that expression of a reporter gene, such as a cat (chloramphenicol acetyl trnasferase) or gus (β-glucuronidase) gene, under the control of an alcA promoter in plants cells, such as maize or tobacco protoplasts, that are incubated with ethanol (inducer) would be dependent on the presence of a polypeptide of the invention.

The invention will now be particularly described in more detail by way of example with reference to the accompanying figures, in which:

FIG. 1. Schematic representation of the alcR gene from Aspergillus nidulans. Amino acids 12-50 represent the DNA binding region, the sequence of which is shown with arrows signifying the position of the degenerate oligonucleotides described in SEQ ID Nos 1 to 8.

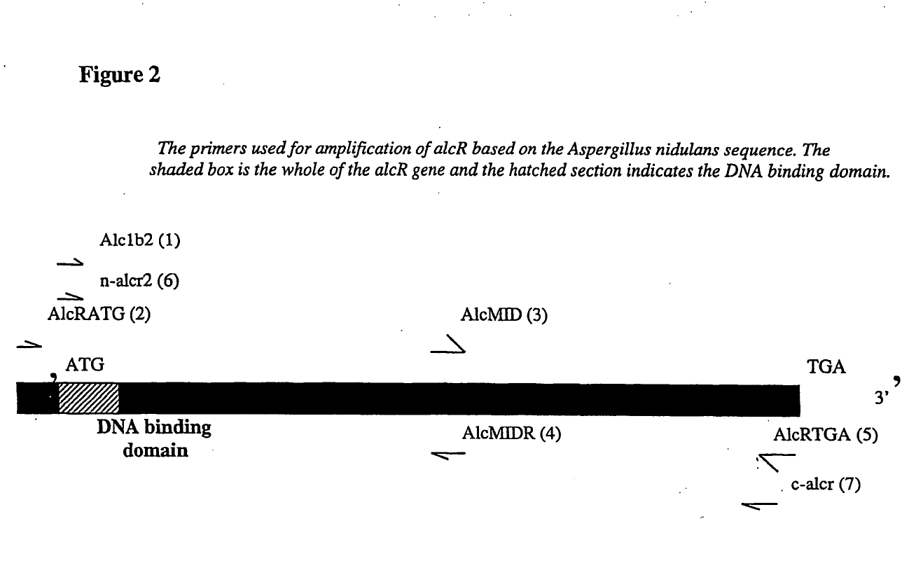

FIG. 2. The position of the degenerate primers used to isolate the alcR sequences from Aspergillus species.

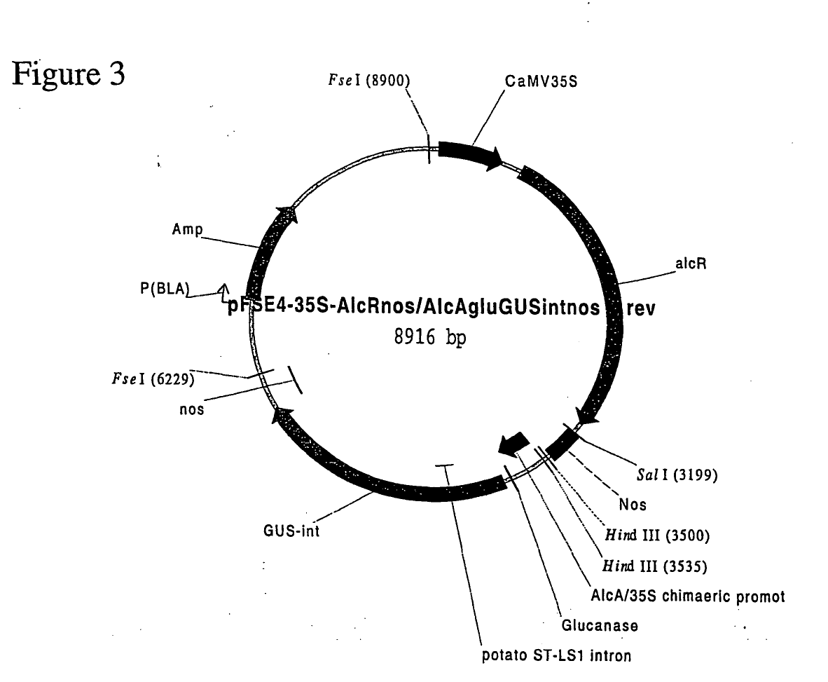

FIG. 3. Plasmid map of progenitor plasmid pFSE4-35S-AlcRnos/AlcAgluGUSintmos-rev.

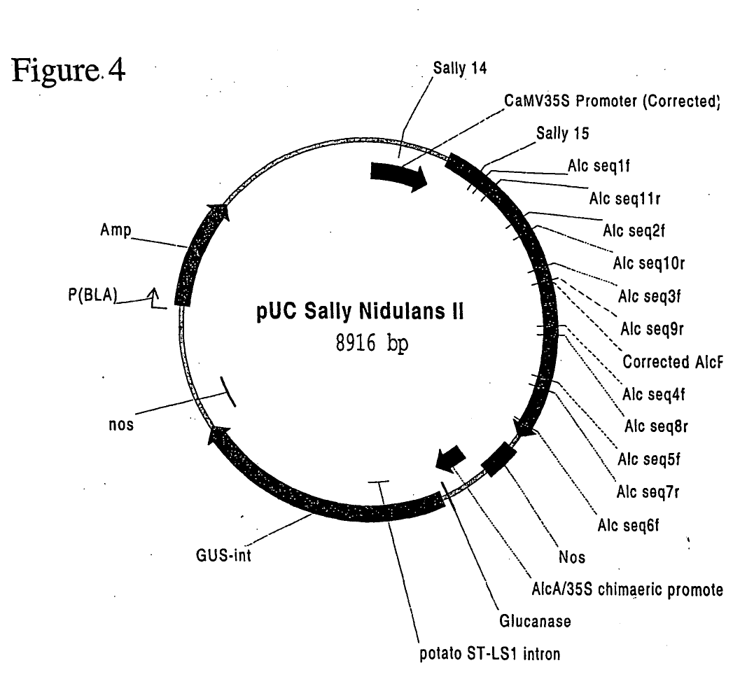

FIG. 4. Plasmid map of pUC Sally Nidulans II vector.

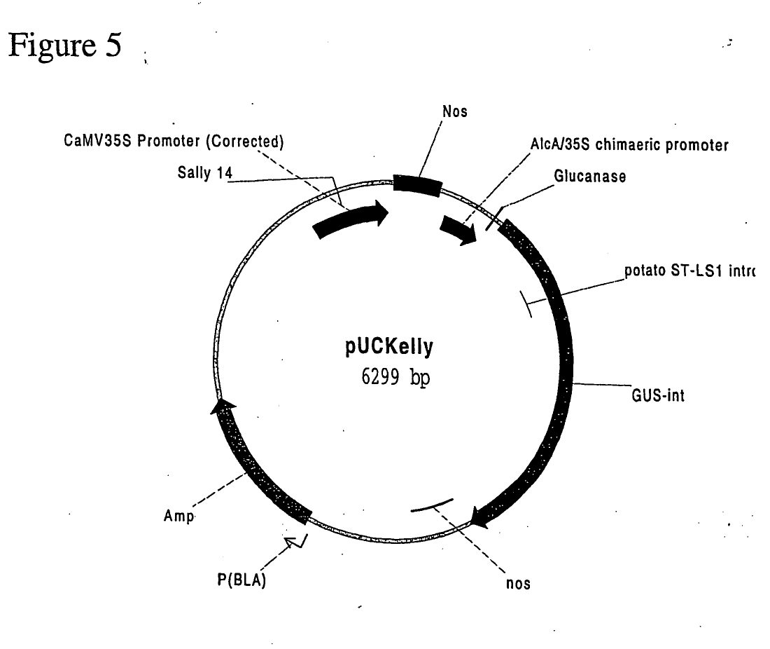

FIG. 5. Plasmid map of pUC Kelly.

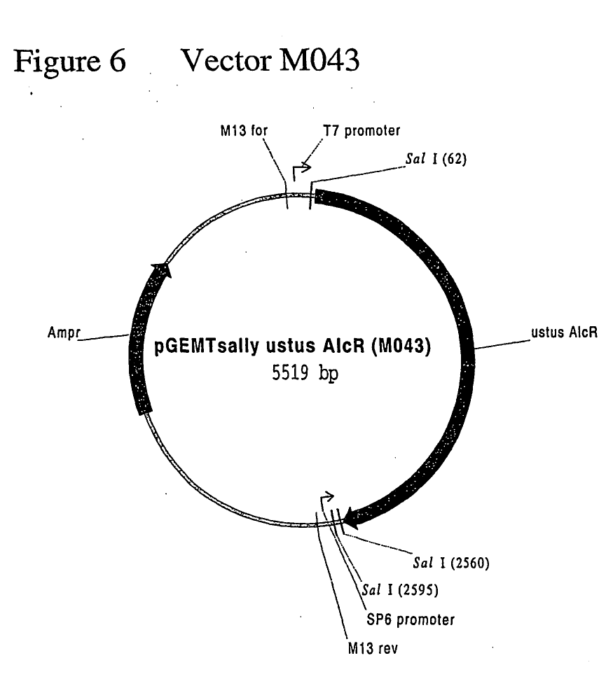

FIG. 6. Plasmid map of vector containing the A. ustus alcR gene, denoted M043.

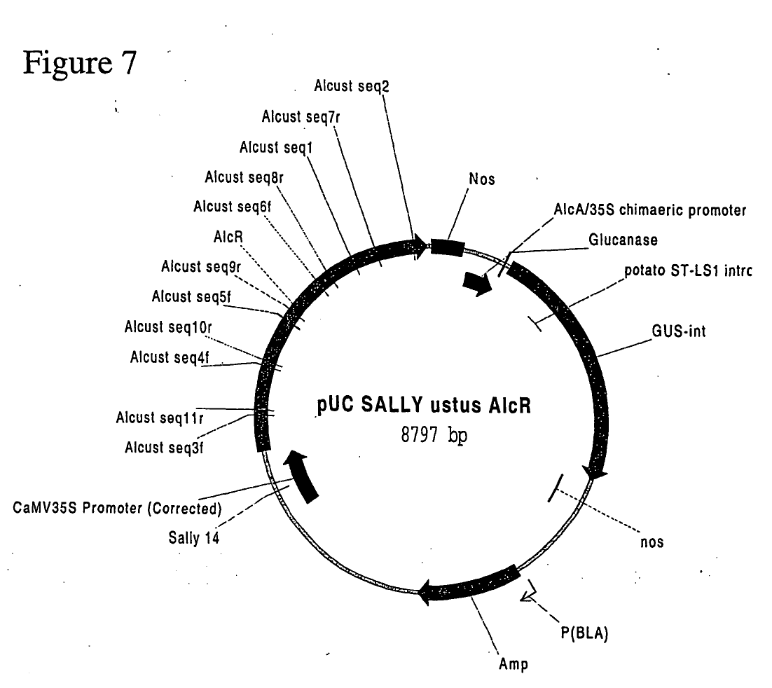

FIG. 7. Plasmid map of pUC Sally containing the alcR Aspergillus ustus gene and named pUC Sally ustus AlcR.

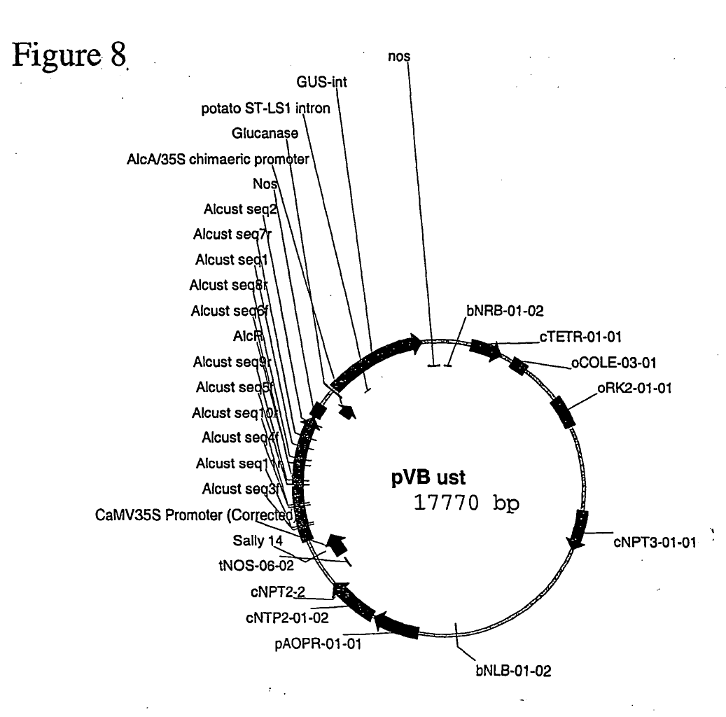

FIG. 8. Plasmid map of the binary vectors containing the both components of the switch. One cassette contains the A. ustus alcR gene under the control of 35S CaMV while the second cassette contains the GUS gene under the control of the alcA inducible promoter. The vector was named pVB Ust.

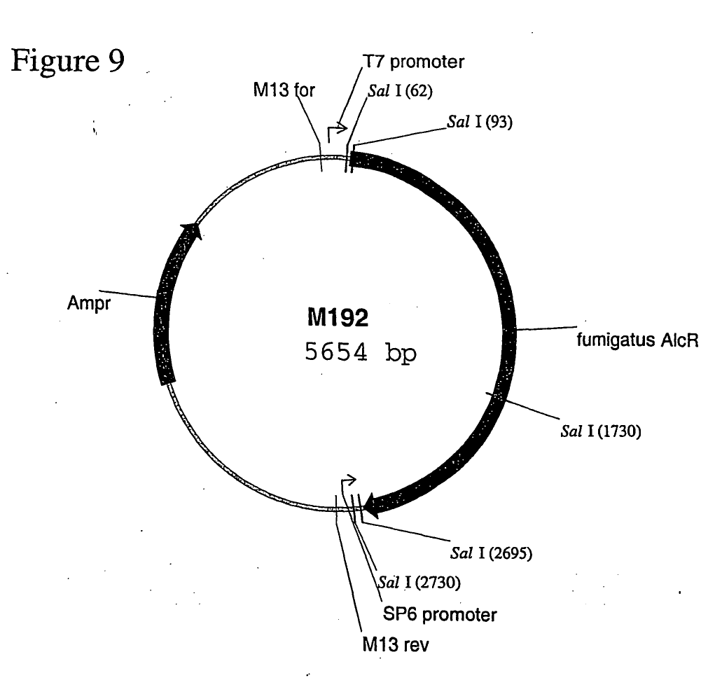

FIG. 9. Plasmid map containing the coding sequence of A. fumigatus alcR gene denotes M192.

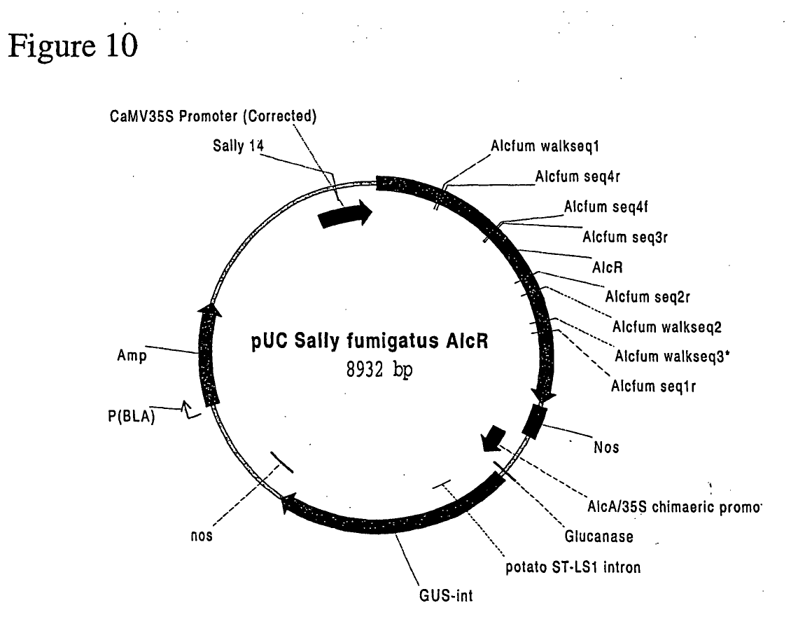

FIG. 10. Plasmid map of pUC Sally containing the Aspergillus fumigatus alcR gene and named pUC Sally fumigatus AlcR.

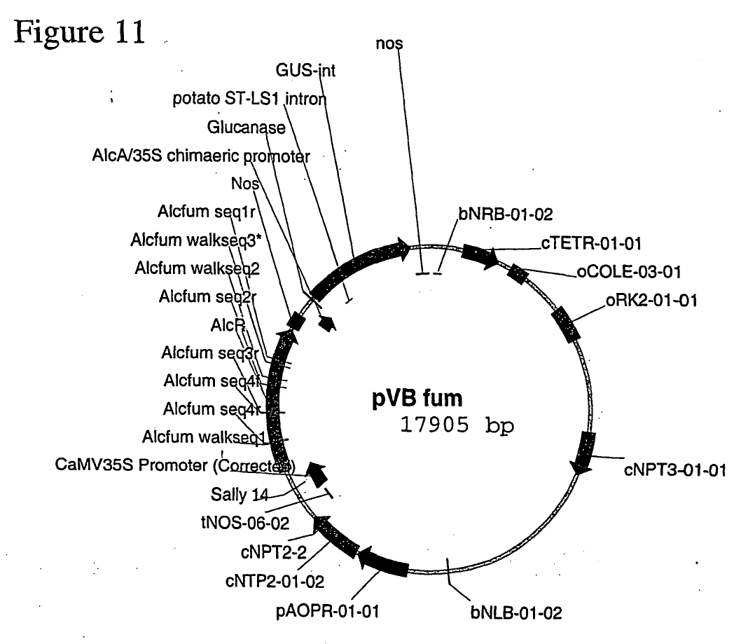

FIG. 11. Plasmid map of the binary vectors containing the both components of the switch. One cassette contains the A. fumigatus alcR gene under the control of 35S CaMV while the second cassette contains the GUS gene under the control of the alcA inducible promoter. The vector was named pVB fum.

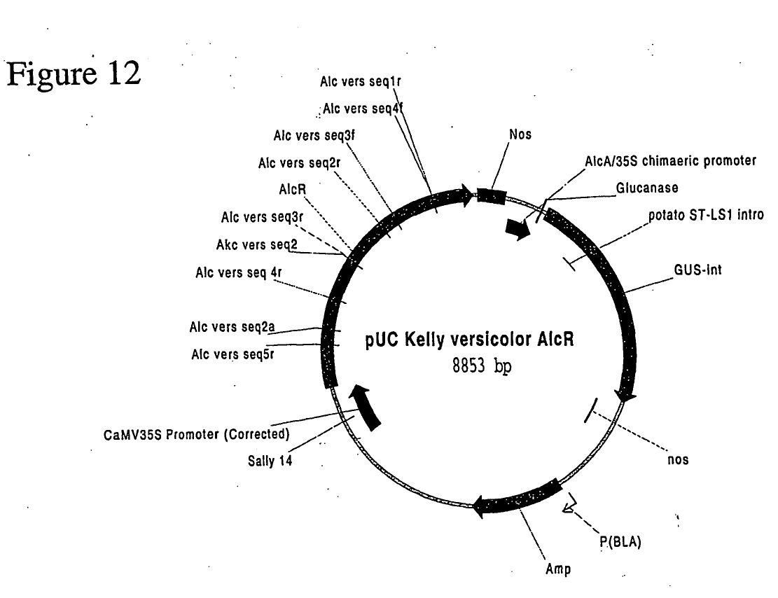

FIG. 12. Plasmid map of pUC Kelly containing the alcR Aspergillus versicolor gene and named pUC Sally versicolor AlcR.

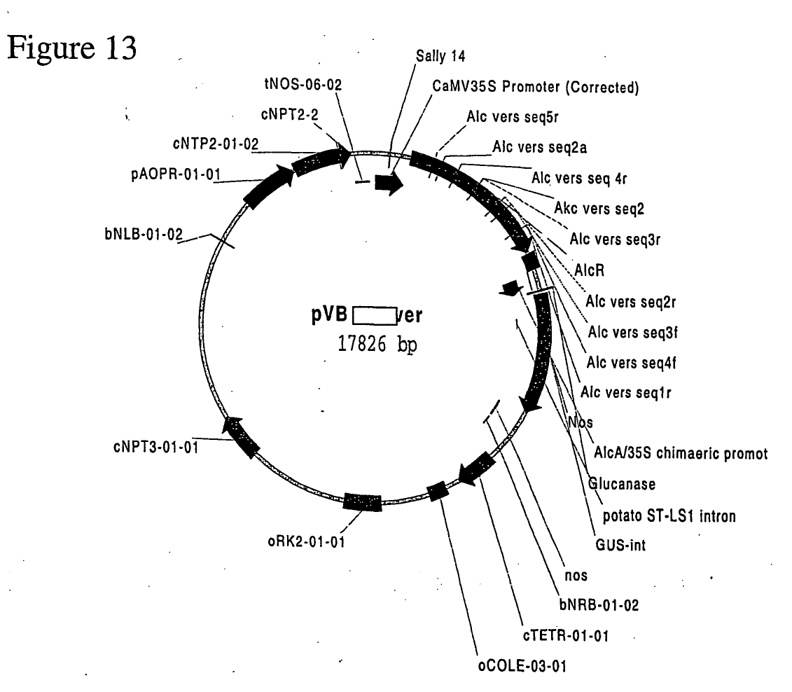

FIG. 13. Plasmid map of the binary vectors containing the both components of the switch. One cassette contains the A. versicolor alcR gene under the control of 35S CaMV while the second cassette contains the GUS gene under the control of the alcA inducible promoter. The vector was named pVB ver.

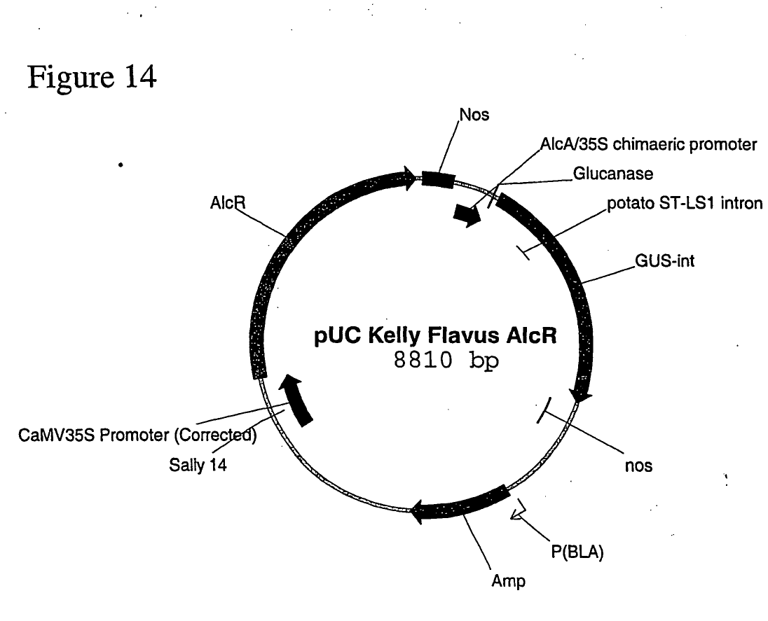

FIG. 14. Plasmid map of pUC Kelly containing the alcR Aspergillus flavus gene and named pUC Kelly flavus AlcR.

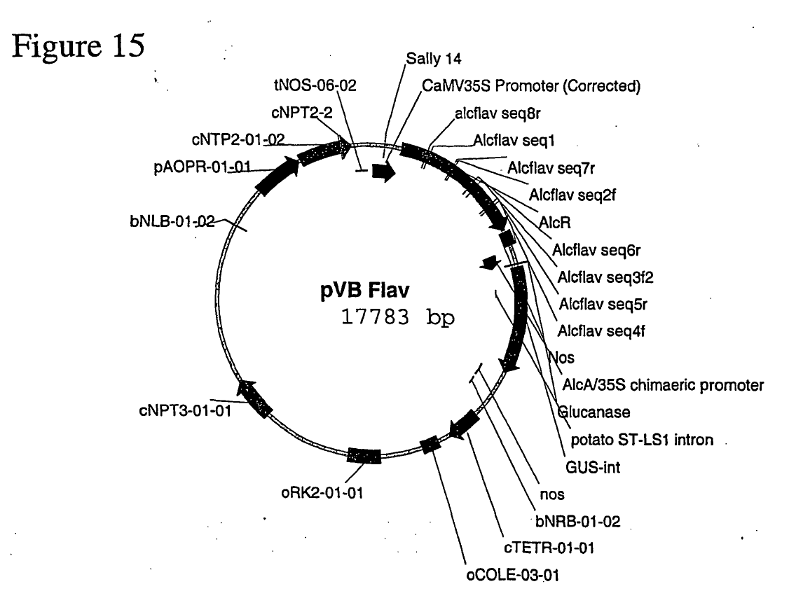

FIG. 15. Plasmid map of the binary vectors containing the both components of the switch. One cassette contains the A. flavus alcR gene under the control of 35S CaMV while the second cassette contains the GUS gene under the control of the alcA inducible promoter. The vector was named pVB Flav.

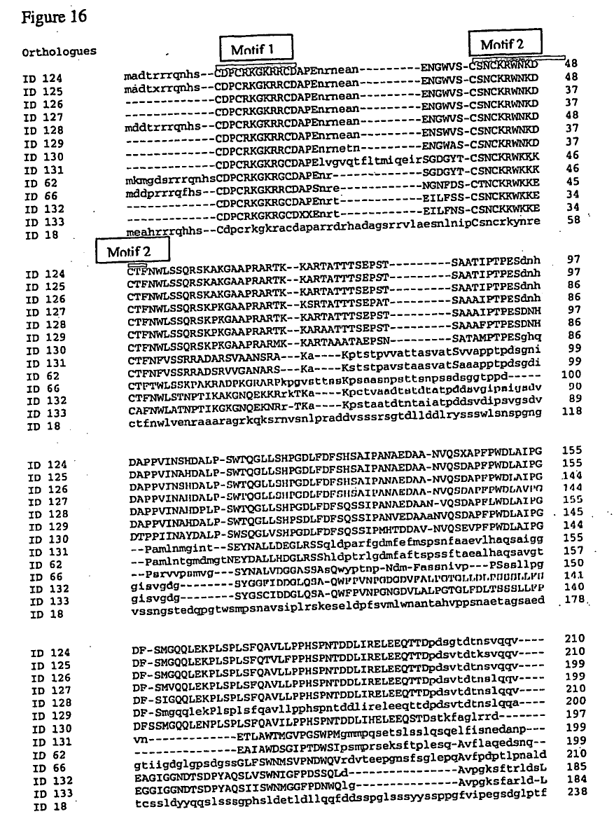

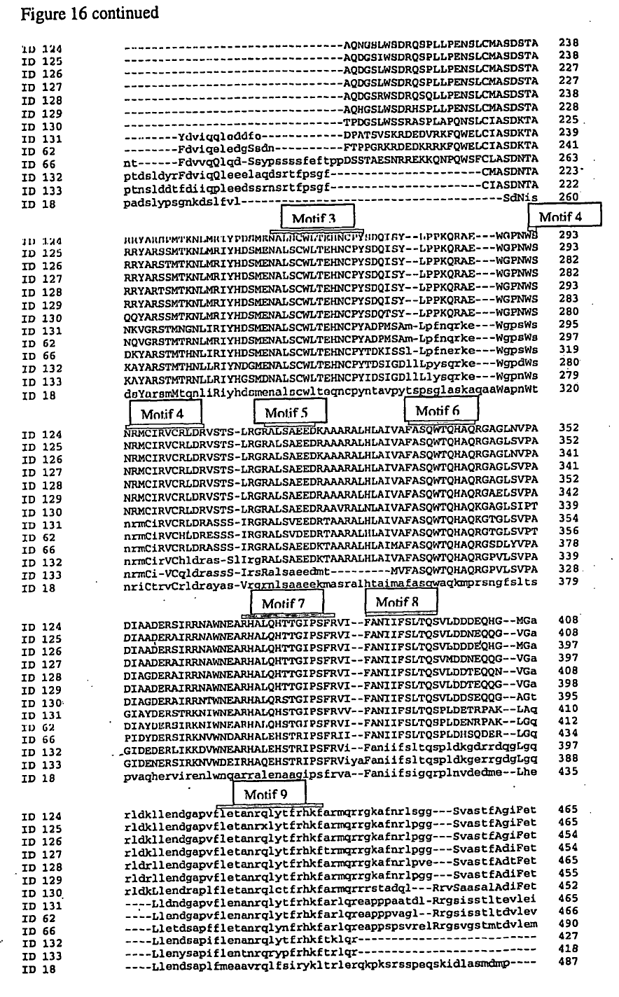

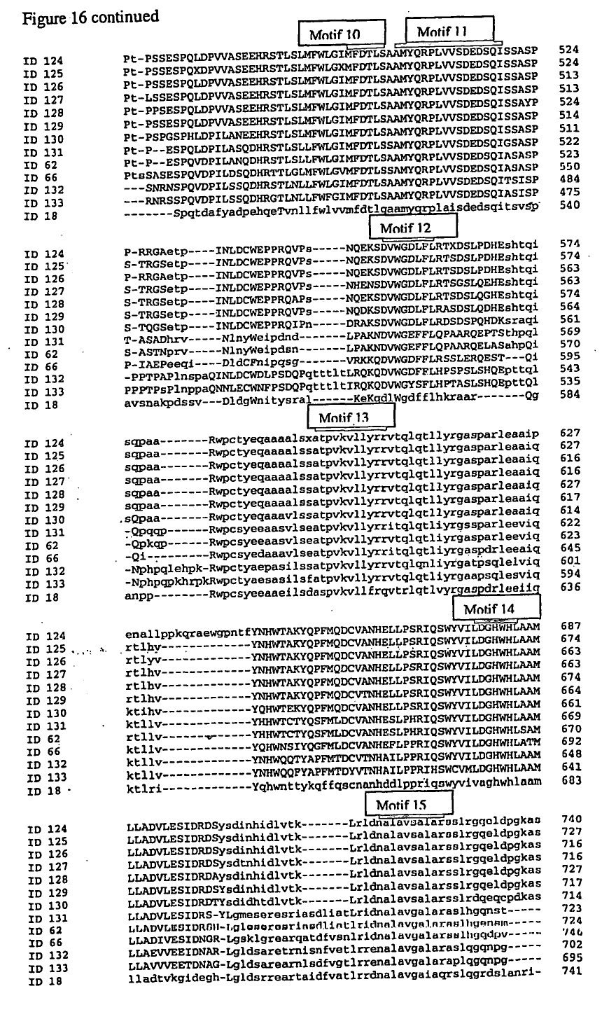

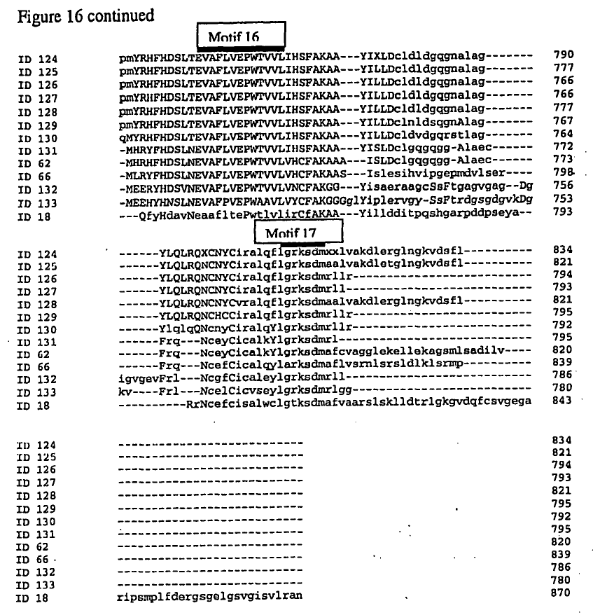

FIG. 16. Alignment of amino acid sequences of AlcR orthologues of the invention showing presence of conserved amino acid motifs.

### EXAMPLE 1

### Isolation of AlcR DNA Binding Domains From Aspergillus Species.

1.1 Isolation of the AlcR DNA Binding Domain Orthologue of A. ustus From Genomic DNA.

Degenerate PCR was carried out on genomic DNA (gDNA) from Aspergillus ustus. Genomic DNA was prepared using either the DNAzol protocol (Helena Biosciences: 0.25 grams ground frozen tissue/0.75 mls DNAzol extraction solution) or the protocol described below:

1. 1g frozen mycellia is grown under liquid nitrogen and added to 15 ml extraction buffer (42% urea, 0.32M NaCl, 50 mM Tris/HCl pH8, 20 mM EDTA pH8, 0.4% N-Lauryl sarcosine).

2. 3 ml phenol pH8 and 3 ml chloroform/isoamyl alcohol (24:1) are added and mixed well

3. sample is centrifuged (10000 rpm, 10 min) and the upper phase transferred to a fresh tube

4. 3 ml 7.5M NH₄Ac and 3.6 ml isopropanol is added and mixed well

5. sample is centrifuged (10000 rpm, 5 min)

6. DNA pellet is washed in 70% EtOH, air dried and resuspended in 200 ul sterile water

PCR is set up using Ready-To-Go PCR beads (Amersham Pharmacia): 22 μl sterile water, 1 μl gDNA and 1 μl each primer (50 μM) as appropriate (see below), 1 PCR bead. Primers (obtained from Life Technologies) to be used: Alc1a2, Alc1b2, Alc1c2, Alc1d2 (forward primers) and Alcrev1a, Alcrev1b, Alcrev1c, Alcrev1d (reverse primers). The PCR is set up using a matrix of forward and reverse primers which is represented by Table I below:

TABLE 1
Matrix of forward and reverse primers for us
in optimising PCR reactions

| | Alc1a2 | Alc1b2 | Alc1c2 | Alc1d2 | Alc1a2 | Alc1b2 | Alc1c2 | Alc1d2 |
|---|---|---|---|---|---|---|---|---|
| Alcrev1a | | | | | | | | |
| Alcrev1b | 48° C. | | | | | | | 55° C. |
| Alcrev1c | | | | | | | | |
| Alcrev1d | | | | | | | | |

Control reactions contain 1 μl gDNA and 1 μl ITS primers (10 μM) or 1 μl A. nidulans gDNA and 1 μl Alc1b2 (50 uM) and 1 μl Alcrev1a (50 uM). PCR is carried out on a Gradient Robocycler (Stratagene) using the following conditions: 94° C. 3 min, (94° C. 1 min, 48-55° C. 1 min, 72° C. 1 min)×35, 72° C. 5 min. 10 ul each reaction is analysed by electrophoresis through a 2% w/v agarose/TBE gel. Fragments of appropriate size are excised and the DNA eluted (using Geneclean Spin Preps, Bio101) and cloned into pCR2.1TOPO (Invitrogen) following the manufacturers protocol. This generates a DNA binding domain having the sequence of SEQ ID NO 9.

1.2 Isolation of Putative DNA Binding Domain of AlcR Orthologue in A. fumigatus From Genomic DNA.

Degenerate PCR is carried out on gDNA from Aspergillus fumigatus. Genomic DNA is prepared using DNAzol ES (Helena Bioscience) as described in 1.1 above. PCR is set up and the oligonucleotides to be used are as described for the isolation of the A. ustus sequence in Example 1.1 above. Control reactions (using 1 μl gDNA and 1 μl ITS primers (10 uM) or 1 μl A. nidulans gDNA and 1 μl Alc1b2 (50 uM) and 1 μl Alcrev1a (50 uM)) are also performed.

PCR is carried out on a Gradient Robocycler (Stratagene) using the following conditions: 4° C. 5 min, (94° C. 1 min, 48-55° C. 1 min, 72° C. 1 min)×35, 72° C. 6 min. 10ul each reaction is analysed by electrophoresis through a 2% w/v agarose/TBE gel. Fragments of appropriate size are excised and the DNA eluted (using Geneclean Spin Preps, Bio101) and cloned into pCR2.1TOPO (Invitrogen) following the manufacturers protocol. Clones that were sequenced yielded the sequence shown as SEQ ID NO 10.

1.3 Isolation of DNA Binding Domain of an AlcR Orthologue in A. versicolor From cDNA.

Degenerate PCR is carried out on cDNA from Aspergillus versicolor. RNA is prepared using TRIzol reagent (Helena Biosciences) following the manufacturers protocol with the following minor amendments:

1. mycellia are ground in liquid nitrogen prior to TRIzol addition,

2. all centrifugation steps are performed at room temperature

cDNA is made using oligo dT and superscript II (Life Technologies) following the protocol supplied with the enzyme. RNaseH digestion is carried out as recommended. PCR isset up using Ready-To-Go PCR beads (Amersham Pharmacia): 22 ul sterile water, 1 ul cDNA and 1 ul each primer (50 uM) as appropriate (see below) plus 1 PCR bead. Primers (obtained from Life Technologies) to be used: Alc1a2, Alc1b2, Alc1c2, Alc1d2 (forward primers) and Alcrev1a, Alcrev1b, Alcrev1c, Alcrev1d (reverse primers). The PCR is set up using a matrix of forward and reverse primers which is represented by Table 2 below:

TABLE 2
Matrix of forward and reverse primers for us in optimising PCR reactions:

| | Alcrev1a | Alcrev1b | Alcrev1c | Alcrev1d | Alcrev1a | Alcrev1b | Alcrev1c | Alcrev1d |
|---|---|---|---|---|---|---|---|---|
| Alc1a2 | | | | | | | | |
| Alc1b2 | 48 C. | | | | | | | 55 C. |
| Alc1c2 | | | | | | | | |
| Alc1d2 | | | | | | | | |

Control reactions are also set up using 1 μl cDNA and 1 μl ITS primers (10 uM) or 1 μA. nidulans gDNA and 1 μl Alc1b2 (50 uM) and 1 μl Alcrev1a (50 uM).

PCR is carried out on a Gradient Robocycler (Stratagene) using the following conditions: 94° C. 3 min, (94° C. 1 min, 48-55° C. 1 min, 72° C. 1 min)×35, 72° C. 6 min. 10 ul each reaction is analysed by electrophoresis through a 2% w/v agarose/TBE gel. Fragments of appropriate size are excised and the DNA eluted (using Geneclean Spin Preps, Bio101) and cloned into pCR2.1TOPO (Invitrogen) following the manufacturers protocol. Sequence showing homology to the DNA binding domain of A. nidulans was obtained (SEQ ID NO 11).

1.4 Isolation of DNA Binding Domain of Aspergillus flavus From Genomic DNA

Degenerate PCR is carried out on gDNA from Aspergillus flavus. Genomic DNA is prepared using DNAzol ES (Helena Bioscience) as described previously (Example 1.1). PCR isset up using Ready-To-Go PCR beads (Amersham Pharmacia): 22 μl sterile water, 1 μl gDNA and 1 μl each primer (50 uM) as appropriate (see below) plus 1 PCR bead. Primers used: Alc1a2, Alc1b2, Alc1c2, Alc1d2 (forward primers) and Alc700rA, Alc700rB, Alc700rC, Alc700rD (reverse primers). The PCR is set up using a matrix of forward and reverse primers which is represented by Table 3 below. Control reactions are also set up using 1 μl gDNA and 1 μl ITS primers (10 uM) or 1 μl A. flavus DNA and 1 μl Alc1b2 (50 uM) and 1 ul Alc700rC (50 uM).

TABLE 3
Matrix of forward and reverse primers for us in optimising PCR reactions:

| | Alc700-rA | Alc700-rB | Alc700-rC | Alc700-rD | Alc700-rA | Alc700rB- | Alc700-rC | Alc700-rD |
|---|---|---|---|---|---|---|---|---|
| Alc1a2 | | | | | | | | |
| Alc1b2 | 48 C. | | | | | | | 55 C. |
| Alc1c2 | | | | | | | | |
| Alc1d2 | | | | | | | | |

PCR is carried out on a Gradient Robocycler (Stratagene) using the following conditions: 94° C. 2 min, (94° C. 30 sec, 48-59° C. 30 sec, 72° C. 1 min 30 sec)×35, 72° C. 10 min. 10 ul each reaction is analysed by electrophoresis through a 2% w/v agarose/TBE gel. Fragments of appropriate size are excised and the DNA eluted (using Geneclean Spin Preps, Bio101) and cloned into pCR2.1TOPO (Invitrogen) following the manufacturers protocol. The DNA sequence of the insert was determined and this is given SEQ ID NO 16.

### EXAMPLE 2

### Isolation of Genomic DNA Encoding AlcR Orthologues

2.1 Isolation of Full Length Genomic DNA Sequence Encoding for A. fumigatus AlcR Sequence

The method of choice for isolating genomic DNA encoding a putative AlcR orthologue uses a genome walking PCR technique as described below. Genomic DNA is prepared using DNAzol ES (Helena Bioscience) as described in Example 1.1.

Creation of “genome walker libraries” is performed using the Clontech Universal Genome Walker Kit following the manufacturers protocol. Primary PCR is set up: 1 Ready-To-Go PCR bead, 22 μl sterile water, 1 μl AP1 primer (genome walker kit), 1 ul GSP1 (at 10 uM), 1 μl appropriate genome walker library (see kit protocol for details). Controls (described in the manufacturers protocol) were also set up with PCR beads. Secondary PCR is set up: 1 PCR bead, 22 ul sterile water, 1 μl AP2 primer (genome walker kit), 1 μl GSP2 (at 10 uM), 1 μl appropriate primary PCR reaction diluted 1:50 in sterile water. Controls (described in the manufacturers protocol) are also set up using Ready-To-Go beads. PCR is carried out using the conditions described in the genome walker protocol, briefly: (94° C. 25 sec, 72° C. 3 min)×7, (94° C. 25 sec, 67° C. 3 min)×32, 67° C. 7 min for primary, (94° C. 25 sec, 72° C. 3 min)×5, (94° C. 25 sec, 67° C. 3 min)×20, 67° C. 7 min for secondary reaction)

10 ul each reaction is analysed by electrophoresis through a 1% w/v agarose/TBE gel. Fragments are excised and DNA eluted using Geneclean Spin Preps (Bio101) and cloned into pCR2.1TOPO (Invitrogen). The process is repeated for completion of both the 5′ and 3′ sequence, thus yielding the open reading frame described in SEQ ID NO 17 (genomic DNA) and SEQ ID NO 18 (predicted amino acid sequence). The 3′ genome walking is carried out twice; in the first instance the GSP1 has the sequence given in SEQ ID NO 12 and GSP2 has the sequence given in SEQ ID NO 19. The second cycle uses GSP1 with the sequence given in SEQ ID NO 20 and GSP2 with the sequence given in SEQ ID NO 21 and SEQ ID NO 22. The whole open reading frame is generated from genomic DNA by amplification with pfu polymerase and oligos with SEQ ID NOs 23 and 24. The derived 5′ sequence encoding the promoter region of the alcR orthologue is disclosed in SEQ ID NO 25. This sequence comprises putative AlcR binding sites providing further evidence in support the identity of the gene (since AlcR is autoregulatory). Only one cycle of genome walking is carried out using GSP1 with the sequence given in SEQ ID NO 26 and GSP2 with the sequence given in SEQ ID NO 27.

2.2 Aspergillus ustus Genome Walking to Isolate Full Open Reading Frame

Isolation of the full open reading frame of an A. ustus alcR orthologue and its promoter region is carried out using the genome walking PCR based method described in example 2.1 above. Genomic DNA is prepared using DNAzol ES (Helena Bioscience) as described in Example 1.1

Creation of “genome walker libraries” is performed using the Clontech Universal Genome Walker Kit following the manufacturers protocol

Primary PCR is set up as described in 2.1 above. Controls use A. fumigatus genome walker library DL4 and primer AF alcgen1. Secondary PCR is also set up as described above (example 2.1). Controls use A. fumigatus genome walker library DL4 and primer AF alcgen2. PCR is performed using the conditions described in the genome walker protocol (see example 2.1) and 10 ul each reaction is analysed by electrophoresis through a 1% w/v agarose/TBE gel. Fragments are excised and DNA eluted using Geneclean Spin Preps (Bio101) and cloned into pCR2.1TOPO (Invitrogen).

For A. ustus the full open reading frame is generated using 3 cycles of genome walking. To obtain the 3′ end of the DNA binding domain sequence (SEQ ID NO 9), SEQ ID NO 28 is used as the GST1 primer and SEQ ID NO 29 is used as GST2, in the first cycle. The second cycle uses SEQ ID 30 as GST1 and SEQ ID 31 as GST2. The third and final cycle uses SEQ ID NO 32 as GST1 and SEQ ID NO 33 as GST2. The 5′ genome walking only requires one cycle of genome walking for which the GST1 primer has SEQ ID NO 34 and GSP2 has SEQ ID NO₃₅. Oligonucleotides (SEQ ID NOs 36 and 37) specific to the beginning and end of the ORF from genomic DNA are used for amplification. The resulting ORF sequence is has SEQ ID NO 38 whilst the promoter sequence has SEQ ID NO 39.

2.3 Aspergillus versicolor Genome Walking to Isolate Full Open Reading Frame

Genomic DNA is prepared as described previously (Example 1.1). An A. versicolor genome walker library is prepared using the Clontech Universal Genome Walker Kit following the manufacturers protocol. Primary PCR is set up as described in 2.1 above. Controls use A. versicolor genome walker library DL4 and primer AF alcgen1. Secondary PCR is also set up as described above (example 2.1). Controls use A. versicolor genome walker library DL4 and primer AF alcgen2. PCR is performed using the conditions described in the genome walker protocol (see example 2.1) and 10 ul each reaction is analysed by electrophoresis through a 1% w/v agarose/TBE gel. Fragments are excised and DNA eluted using Geneclean Spin Preps (Bio101) and cloned into pCR2.1TOPO (Invitrogen).

To isolate the 3′ end portion of the A. versicolor gene one cycle of genome walking is performed using SEQ ID NO 40 as GST1 and SEQ ID NO 41 as GST2. One cycle of genome walking is required to complete the 5′ end of the gene and provide sequence of the promoter region. This is achieved by using SEQ ID NO 42 as GST1 and SEQ ID NO 43 as GST2. Oligonucleotides (SEQ ID NOs 44 and 45) are used to amplify the whole of the A. versicolor alcR ORF with pfu polymerase. The resulting fragment has the sequence identified as SEQ ID NO 46, with the sequence of the promoter region being given SEQ ID NO 47. This sequence comprises putative AlcR binding sites providing further evidence in support the identity of the gene (since AlcR is autoregulatory).

2.4 Aspergillus flavus Genome Walking to Isolate Full Open Reading Frame

Isolation of the full open reading frame of an A. flavus alcR orthologue and its promoter region is carried out using the genome walking PCR based method described in example 2.1 above. Genomic DNA is prepared using DNAzol ES (Helena Bioscience) as described in Example 1.1

Creation of “genome walker libraries” is performed using the Clontech Universal Genome Walker Kit following the manufacturers protocol

Primary PCR is set up as described in 2.1 above. Controls use A. flavus genome walker library DL4 and primer AF alcgen1. Secondary PCR is also set up as described above (example 2.1). Controls use A. flavus genome walker library DL4 and primer AF alcgen2. PCR is performed using the conditions described in the genome walker protocol (see example 2.1) and 10 ul each reaction is analysed by electrophoresis through a 1% w/v agarose/TBE gel. Fragments are excised and DNA eluted using Geneclean Spin Preps (Bio101) and cloned into pCR2.1TOPO (Invitrogen).

In order to generate the 3′ end sequence of the A. flavus alcR orthologue, 3 cycles of genome walking are required. Cycle 1 uses SEQ ID NO 48 as the GST1 primer and SEQ ID NO 49 as GST2; Cycle 2 uses SEQ ID NO 50 as the GST1 primer and SEQ ID NO 51 as GST2; Cycle 3 uses SEQ ID NO 52 as the GST1 primer and SEQ ID NO 53 as GST2. The 5′ end is generated using a single cycle of genome walking which also generates promoter sequence for the gene. This cycle uses SEQ ID NO as GST1 and SEQ ID NO 55 as GST2. The whole ORF is amplified bay PCR with primers having SEQ ID NOs 56 and 57 thus resulting in a DNA fragment encoding the full-length alcR orthologue from A. flavus (SEQ ID NO 58). The predicted amino acid sequence of this AlcR orthologue is given SEQ ID NO 59. The promoter sequence of the alcR orthologue (sequence ID 60) contains sequences with identity to tie AlcR binding sites observed in A. nidulans.

### EXAMPLE 3

### Isolation of alcR cDNA Othologues

3.1 Isolation of cDNA Encoding the AlcR Orthologue of A. ustus.

RNA is extracted as described in Example 1.3 and the first stand cDNA is generated using Superscript (Life Technologies). Reverse transcription is carried out with the oligodT primer supplied by Promega in the kit. The cDNA is used as a template for a PCR reaction in which the DNA fragment generated lacks any potential introns. PCR conditions: 95° C. 10 min (95° C. 30 sec, 55° C. 30 sec, 72° C. 1.5 min)×35, 72° C. 10 min). PCR primers used have SEQ ID NOs 36 and 37. The product lacks sequence when compared to the genomic DNA version of the alcR orthologue in A. ustus. Furthermore, the intron sequence is where predicted. The sequence of the full length ORF lacking intron sequences has SEQ ID NO 61. This encodes a putative polypeptide product having the amino acid sequence given SEQ ID NO 62.

3.2 Isolation of cDNA Encoding the AlcR Orthologue of A. versicolor.

RNA is extracted as described above and then first stand cDNA is generated using using Superscript (Life Technologies). An alcR sequence specific primer (SEQ ID NO63) is used for cDNA generation. cDNA is used as a template for a PCR reaction to generate a DNA fragment lacking potential intron sequences. PCR primers used have SEQ ID NOs 44 and 64. The product lacks sequence when compared to alcR from A. nidulans. Furthermore, the intron sequence is found where. The sequence of the full length ORF lacking intron sequences has SEQ ID NO 65. This encodes a putative polypeptide product having the amino acid sequence given SEQ ID NO 66.

### EXAMPLE 4

### Analysis of Possible Introns in alcR Orthologues

The Aspergillus nidulans alcR gene contains a single intron, 75 bases from the ATG start codon, in the middle of the DNA binding domain. This intron is 60 bp in length and hence in frame. Analysis of the gDNA sequences of the Aspergillus orthologue genes, revealed the following:

1) the A. versicolor gene must contain an intron as the two halves of the DNA binding domain are out of frame

2) the A. ustus gene must contain an intron as the two halves of the DNA binding domain are out of frame

3) the A. fumigatus gene may or may not contain an intron as the two halves of the DNA binding domain are in frame

4) the A. flavus gene may or may not contain an intron as the two halves of the DNA binding domain are in frame

To ascertain where the introns are in A. versicolor and A. ustus, and whether there are introns in A. fumigatus and A. flavus, RT PCR is carried out.

4.1 Identification of Intron Boundaries in alcR of A. versicolor

To obtain RNA, Aspergillus versicolor cultures are grown in potato dextrose media (20 ml Glycerol, 10 g Yeast Extract, 0.5 g MgSO₄.7H₂O, 6.0 g NaNO₃, 0.5 g KCl, 1.5 g KH₂PO₄ made up to 1 litre with water) for 5 days at 24° C. Cultures are filtered through myra cloth to collect the mycelia and flash frozen in liquid nitrogen. Frozen mycelia are ground in a pestle and mortar under liquid nitrogen. Ground mycelia are added to 0.75 ml TRIzol reagent (Life Technologies). Chloroform (0.75 ml) is added and the mixture shaken at room temp for 5 min. Following centrifugation (13,000 rpm, 15 min) the top phase is transferred to a fresh tube and 1 ml isopropanol added and mixed thoroughly prior to incubation at room temperature for 10 min. Following centrifugation (13,000, 10 min), the supernatant is removed and 2 ml 70% EtOH added. This is again mixed thoroughly and centrifuged for a further 10 min at 13,000 rpm. The supernatant is discarded, and the DNA pellet air dried and resuspended in 50 ul DEPC treated H₂O.

Reverse transcription is set up as follows: 1 ul oligo (SEQ ID NO 134), 1 ul RNA, 12 ul H₂O. This mixture is incubated at 70° C. for 10 min and then returned to ice. Following the addition of 5 ul first strand buffer, 2 ul DIT and 1 ul dNTP, the mixture is incubated at 42° C. for 2 min. Following this incubation, 1 ul superscript is added and the mixture incubated at 42° C. for 50 min, followed by 15 min at 72° C. and 1 hr at 4° C.

PCR is set up as follows: 6 ul reverse transcription reaction, 2.5 ul oligol (SEQ ID NO145; 10 uM), 5 ul oligo 2 (SEQ ID NO 136; 5 uM), 17.5 ul H₂O plus 1 Ready-To-Go PCR bead. PCR conditions are: 95° C. 10 min (95° C. 1 min, 55° C. 1 min, 72° C. 3 min)×35 cycles, 72° C. 10 min. Samples are analysed by electrophoresis through 1% w/v agarose TBE gel and fragments purified using Geneclean spin kit (Bio101). Purified products are cloned into pCR2.1 TOPO (Invitrogen) following the manufacturers protocol. Ligated TOPO products are transformed into Escherichia coli TOP10 cells (3 ul DNA added to cells on ice and left for 30 min, placed at 42° C. for 90 sec then returned to ice for 2 min. 25° ul SOC media added and shaken at 37° C. for 1 hr. 100 ul cells plated onto LB+amp plates and left to grow at 37° C. overnight).

Colonies are screened by PCR: 23 ul water, 1 PCR bead 1 ul primer (SEQ ID NO 91; 5 pmol/ul)+1 ul primer (SEQ ID NO 92; 5 pmol/ul). Colonies are used to inoculate PCR mix and to prepare LB+amp streak plates. Plates are incubated at 37° C. for 2 days. PCR is carried out on Biometra Tgradient PCR machine using the following conditions: 95° C. 10 min, (95° C. 30 sec, 55° C. 30 sec, 72° C. 2 min) 35 cycles, 72° C. 10 min. Reactions are analysed by electrophoresis through 1% w/v agarose 113E gel. Colonies showing the correct sized fragment are used to prepare small scale cultures form which DNA is prepared (using Qiagen Spin minipreps). Analytical EcoRI (NEB) restriction digests are carried out to confirm the PCR results. Clones showing the correct banding pattern are sequenced. These clones show a 65 bp deletion when compared with the gDNA clone. Analysis of the predicted amino acid sequence shows that the two halves of the DNA binding domain are in frame. One such clone is designated pCR2.1 Asp-vers-AlcR-RTfrag.

To obtain a full-length clone containing the sequence from cDNA, the RT fragment is spliced onto the 3′end of the A. versicolor gDNA (which is the same as cDNA as there are no further introns in the gene). pCR2.1 Asp-vers-AlcR-RTfrag is digested with SpeI (37° C., 3 hrs) and pCR2.1 vers AlcR gDNA was digested with SpeI and XbaI (37° C., 3 hrs). Reactions are analysed on 1% agarose gel and the required bands were purified using the Geneclean spin kit (Bio101). The pCR2.1 Asp-vers-AlcR-RTfrag fragment is phosphatased using shrimp alkaline phosphatase (SAP; 37° C., 1 hr) and the DNA cleaned using the Geneclean spin kit (Bio101). Ligations are set up using 6 ul phosphatased RTfrag vector and 2 ul insert (T4 DNA ligase, 16° C., 16 hrs). TOP10 cells are transformed with 3 ul of each ligation reaction, as described, previously.

Colonies are screened by PCR as described above, but using primers with SEQ ID NOs 92 and 136. Colonies showing the correct sized fragment are used to prepare small scale cultures form which DNA is prepared (using Qiagen Spin minipreps). Analytical EcoRI (NEB), and XbaI/SpeI (NEB) restriction digests are carried out to confirm the PCR results. Clones showing the correct banding pattern are sequenced to verify insertion of the full-length cDNA sequence. Clones carrying the full-length sequence are designated pCR2.1 Asp-vers-AlcR-flcDNA.

4.2 Identification of Intron Boundaries in the DNA Binding Domain of the AlcR from A. ustus

Cultures for isolation of RNA from A ustus are grown and the RNA extracted as described for A. versicolor (Example 4.1). Reverse transcription is set up (1 ul oligo dT₍₁₅₎, 5 ul RNA, 7 ul H₂O and carried out as described above.

PCR is set up as follows: 6 ul reverse transcription reaction, 2 ul oligo 1 (SEQ ID NO 137; 10 uM), 2 ul oligo 2 (SEQ ID NO 138; 10 uM), 21 ul H₂O, 1 PCR bead and carried out under the following conditions: 95° C. 10 min (95° C. 1 min, 55° C. 1 min, 72° C. 3 min)×35 cycles, 72° C. 10 min. Samples are analysed by electrophoresis through 1% w/v agarose TBE gel and fragments purified using Geneclean spin kit (Bio101). Purified products are cloned into pCR2.1 TOPO (Invitrogen) following the manufacturers protocol. Ligated TOPO products are transformed into TOP10 cells, as described previously.

Colonies are screened by PCR (using primers with SEQ ID NOs 91 and 92) as is described in example 4.1 above. Colonies showing the correct sized fragment are used to prepare small scale cultures form which DNA is prepared (using Qiagen Spin minipreps). Analytical EcoRI (NEB) restriction digests are performed and clones showing the correct banding pattern are sequenced. These clones show an approx. 100 bp deletion when compared with the gDNA clone. Analysis of the predicted amino acid sequence shows that the two halves of the DNA binding domain are in frame. However, these clones also showed other differences from the gDNA sequence, which are thought to be due to the PCR using Taq polymerase. These clones are designated pCR2.1 Asp-ust-AlcR-cDNA*.

To obtain a clone containing the correct sequence, the 5′ end of the cDNA is spliced onto the 3′end of A. ustus gDNA (which is the same as cDNA as there are no further introns in the gene). pCR2.1 Asp-ust-AlcR-cDNA* and pCR2.1 ust AlcR gDNA are digested with BamHI (37° C., 3 hrs). Reactions are analysed, DNA fragments purified as described above. The pCR2.1 Asp-ust-AlcR-cDNA* fragment is phosphatased using SAP (37° C., 1 hr) and the DNA cleaned using the Geneclean spin kit (Bio101). Ligations are set up using 6 ul phosphatased RTfrag vector and 2 ul insert (T4 DNA ligase, 16° C., 16 hrs). TOP10 cells are transformed as described previously.

Colonies are screened by PCR (using primers with SEQ ID NOs 92 and 138) as described in example 4.1 above. Colonies showing the correct sized fragment are used to prepare small scale cultures from which DNA is prepared (using Qiagen Spin minipreps). The DNA is sequence verified to check the presence of the corrected sequence. Clones carrying the correct A. ustus cDNA sequence are designated pCR2.1 Asp-ust-AlcR-flcDNA.

4.3 Identification of Intron Boundaries Within the DNA Binding Domain of AlcR From A. flavus

Cultured from A. flavus are grown and RNA extracted as described for A. versicolor. RNA is treated with DNase (RQ1 DNase plus RNasin at 37° C., 1 hr) and cleaned up using the RNeasy Kit (Invitrogen). The RNA is diluted 1:5 in DEPC treated H₂O, re-treated with DNase and cleaned up using the RNeasy Kit. RT PCR is carried out using the 5′ RACE kit from Ambion. RNA is phosphatased (CIP), cleaned up using the RNeasy kit and treated with TAP according to the manufacturers instructions. It is then ligated to the RNA RACE adapter following the manufacturers protocol.

Reverse transcription is set up and performed as described above, but using primer having SEQ ID NOs139. Control reactions containing all components except superscript are also set up and taken through the PCR steps.

Primary PCR reactions are set up containing: 2 ul reverse transciption reaction, 2 ul oligo 1 (SEQ ID NO 140; 10 uM), 2 ul oligo 2 (SEQ ID NO 141; 10 uM); 19 ul H₂O, 1 PCR bead. Primary PCR is carried out, and the samples analysed as described in example 4.2. Primary PCR is diluted with 245 ul Tricine EDTA. Secondary PCR is then set: 5 ul diluted primary PCR, 2 ul oligo 1 (SEQ ID NO 140), 2 ul oligo 2 (SEQ ID NO 142), 16 ul H₂O, 1 PCR bead. PCR is performed under the conditions described in example 4.1). PCR reactions are analysed by agarose gel electrophoresis as described previously. Samples show a band clearly visible in the RT lane that is not present in the control lacking superscript. This band is purified and cloned into pCR2.1 TOPO as described previously. TOP10 cells are transformed with the ligation reactions.

Colonies are screened by PCR (using primers with SEQ ID NOs 91 and 92) as described in example 4.1 above. Colonies showing the correct sized fragment are used to prepare small scale cultures form which DNA is prepared (using Qiagen Spin minipreps). These are sequenced. Clones show the same sequence as the gDNA clone confirming that the Aspergillus flavus alcR orthologue does not contain an intron.

4.4 Identification of Intron Boundaries in the alcR Orthologue of A. fumigatus

Cultures of Aspergillus fumigatus are grown and RNA extracted and prepared as described for A. flavus (example 4.3).

Reverse transcription reactions contain: 1 ul oligo dT₍₁₅₎, 1 ul RNA, 11 ul H₂O and are carried out with appropriate controls as described in example 4.1.

PCR reactions contain: 1 ul reverse transcription reaction+1 ul oligo 1 (SEQ ID NO 143; 10 uM), 2 ul oligo 2 (SEQ ID NO 144; 10 uM), 22 ul H₂O,1 PCR bead. PCR is performed under the conditions described in example 4.1. PCR reactions are analysed by agarose gel electrophoresis as described previously. Samples show a band clearly visible in the RT lane that is not present in the control lacking superscript. This band is purified and cloned into pCR2.1 TOPO as described previously. TOP10 cells are transformed with the ligation reactions.

As before, colonies are screened by PCR using primers having SEQ ID NOs 91 and 92.: Colonies showing the correct sized fragment are used to prepare small scale cultures form which DNA is prepared (using Qiagen Spin minipreps). These clones are sequenced and show the same sequence as the gDNA clone confirming that the Aspergillus fumigatus alcR does not contain an intron.

### EXAMPLE 5

### Isolation of alcR Orthologues From Other Aspergillus Species

Different Aspergillus species and sub-species were chosen from a variety of geographical locations. Degenerate oligonucleotides are selected after the open reading frame sequences of the A. ustus, A. versicolor, A. Fumigatus and A. flavus are aligned. Consensus regions that allowed the longest possible fragment to be isolated from one PCR reaction are generated (SEQ ID NO 2 Alc1b2, SEQ ID NO 67,n-alcr2 and SEQ ID NO 68, c-alcr). A second set of degenerate oligonucleotides spanning the whole of the coding sequence is also generated (SEQ ID NO 69, AlcRATG and SEQ ID NO 70, alcRTGA). Finally, in case a 2.4 Kb fragment proved difficult to isolate via PCR, a set of degenerate oligonucleotides is produced from a consensus sequence laying in the middle of the gene (SEQ ID NO 71, alcMID and SEQ ID NO, alcMIDR). FIG. 2 shows the relative location of the oligonucleotides to the coding sequence of the gene. Table 4, shows the species from which genes were isolated and the degenerate oligonucleotides used to generate the DNA fragments.

For standard PCR using taq DNA polymerase, Ready-To-Go PCR beads (Amersham Pharmacia Biotech) are used. When brought to a final volume of 25 μl, each reaction contains; 1.5 units taq DNA polymerase, 10 mM Tris-HCL, (pH 9.0 at room temperature), 50 mM KCl, 1.5 mM MgCl, 200 μM of each dNTA and stabilisers, including BSA. Added to each PCR bead is 1 μl of genomic DNA, and 1 μl of each primer (10 μp). The tubes are then placed into a PCR machine and the DNA denatured for 5 minutes at 95° C. 35 cycles of amplification is then performed. Each cycle consists of 94° C. for 1 minute, melting temperature (Tm) for 1 minute, and 72° C. for 1.5 minutes, followed by a further 10 minutes at 72° C. The Tm varies, depending on the primers used. A temperature gradient is used for some primer combinations because the Tm of the two primers is different.

Higher fidelity DNA polymerase enzymes are used to reduce the error rate of replication. pfu turbo has the lowest error rate of most DNA polymerases (1.3×10⁻⁶) and is used to amplify ITS regions from Aspergillus species. 4 μl pfu turbo 10× buffer, 4 μl 2.5 mM dNTP mix, 1 μl DNA template, 1 μl ITS1, 1 μl ITS4, and 27 μl distilled water are added to an Eppendorf tube. The tube is placed into a PCR machine at 94° C. for 2 minutes to denature the DNA. 20 of pfu turbo is then added to the tube and 35 cycles of amplification are performed. Each cycle consists of 94° C. for 30 seconds, 55° C. for 30 seconds, and 72° C. for 1 minute. The 35 cycles are followed by a further 10 minutes at 72° C. Amplified DNA fragments are fractionated using a 1%(w/v) agarose gel made with 1×TBE. The fragments are visualised using a UV trans-illuminator and cut out of the gel and purified using QIAquick Gel Extraction Kit from QIAGEN. The products are then subcloned into PCR2.1TOPO vector (InvitroGen) and positive clones screened by PCR. The positive clones are grown in small scale culture and the DNA extracted and its determined. The sequence ID for the different isolated sequences is provided in Table 4.

TABLE 4
The combination of primers used to amplify alcR genes
from Aspergillus species. Not all the primers work with
all species so combinations are used until an alcR
gene is amplified.

| | Forward | | |
|---|---|---|---|
| Species | primer | Reverse primer | Sequence ID |
| Aspergillus bicolor | Alc1b2 | c-alcr | 73 |
| Aspergillus corrugatus | AlcRATG | AlcMIDR | 74 |
| (2 fragments) | AlcMID | AlcRTGA | |
| Aspergillus cleistominutus | n-alcr2 | c-alcr | 75 |
| Aspergillus foveolatus | n-alcr2 | c-alcr | 76 |
| Aspergillus heterothallicus | Alc1b2 | c-alcr | 77 |
| Aspergillus navahoensis | n-alcr2 | c-alcr | 78 |
| Aspergillus spectabilis | Alc1b2 | c-alcr | 79 |
| Aspergillus nidulans variant | AlcRATG | AlcMIDR | 101 |
| acristatus | AlcMID | AlcRTGA | |
| (2 fragments) | | | |
| Aspergillus nidulans variant | AlcRATG | AlcMIDR | 102 |
| dentatus | AlcMID | AlcRTGA | |
| (2 fragments) | | | |
| Aspergillus nidulans variant | Alc1b2 | c-alcr | 103 |
| vuillemin | | | |

### EXAMPLE 6

### Production of Expression Vectors for Plant Transformation Containing the Isolated DNA Sequences from A. ustus, A. versicolor, A. fumigatus and A. flavus.

6.1 Creation of Intermediate Vectors for Cloning

6.1.1 Creation of pUC Sally nidulans II Intermediate Vector

The pFSE4-35S-AlcRnos/AlcAgluGUSintnos b rev vector (FIG. 3) contains all the components needed for the intermediate cloning of the alcR orthologues from A. ustus, A. fumigatus, A. versicolor and A. flavus. However, there are no suitable restriction sites. Therefore, site directed mutagenesis (SDM) is used to create a second SalI site in the vector. SDM is performed using the Quikchange SDM kit (Stratagene). Cycling is set up using 1 ul template DNA and 125 ng Sally 17P (SEQ ID NO 99) and Sally18P (SEQ ID NO 100) primers following the manufacturers protocol. Cycling conditions are: 95° C. 30 sec, (95° C. 30 sec, 55° C. 1 min, 68° C. 18 min)×14 cycles. Reactions are then digested with DpnI (37° C., 1 hr) and transformed into E. coli XL1-Blue cells (transformation is carried out as described previously for TOP10 cells).

Colonies are used to prepare cultures from which DNA is prepared. SalI restriction digests are performed on the DNA (37° C., 45 min) and analysed on a 1% agarose gel. Clones showing the correct banding pattern are sequence verified. This vector is designated pUC Sally nidulans II (FIG. 4).

6.1.2 Creation of pUC Kelly Intermediate Vector

The pUC Sally nidulans II vector is digested with SalI (37° C., 3 hrs) and analysed on a 1% gel. The large backbone plasmid band is purified using Bio101 geneclean spin. This plasmid is then self-ligated (1 ul DNA, 1 ul buffer, 1 ul T4 DNA ligase at 16° C. for 16 hrs). The DNA is re-transformed into TOP10 competent cells (as described previously). Colonies are used to prepare cultures from which DNA is prepared. XmaI (NEB) restriction digests are performed on the DNA (37° C., 2 hrs) and these are analysed on 1% agarose gel. Clones showing correct banding pattern are sequence verified. This vector is designated pUC Kelly (FIG. 5).

6.2 Construction of Orthologue Specific Expression Vectors

6.2.1 Expression Vector for the Expression of A. ustus alcR Sequence

(NB All AlcRs were cloned into pCR2.1 following amplification from genomic or cDNA)

The alcR gene is amplified by PCR from pCR2.1 using primers Sally Three (SEQ ID NO 80) and Sally Four (SEQ ID NO 81), adding SalI sites to both 3′ and 5′ ends (1 ul pCR2.1 ustus AlcR, 1 ul each primer at 25 pmol/ul, 10 ul buffer, 0.2 ul 100 mM dNTP mix, 1 ul pfu Turbo made up to 50 ul with H₂O). PCR conditions are: 95° C. 5 min, (95° C. 1 min, 55° C. 1 min, 72° C. 3 min)×40 cycles, 72° C. 10 min. This PCR product is cloned into pGEM-Teasy (Promega) following the manufacturers protocol. The ligated product is transformed into E. coli DH5α cells (transformation is carried out as described previously for TOP10 cells). Colonies are analysed by restriction digestion with SalI. Clones producing the expected restriction pattern were designated M043 (FIG. 6). M043 is digested with SalI (37° C., 1 hr) and the required band was purified using Bio101 Geneclean spin kit. pUC Sally nidulans II is also digested with SalI (37° C., 45 min), then phosphatased using SAP (37° C., 45 min). The reaction is analysed on 1% agarose gel and the required vector backbone purified using the geneclean spin kit (Bio101). Ligations are then set up using 7 ul alcR fragment and 1 ul phosphatased vector (T4 DNA ligase, 16 hrs, 16° C.) and the DNA is transformed into DH5α competent cells.

Colonies are screened by PCR: 23 ul water, 1 Ready-To-Go PCR beads, 1 ul SALLY14 primer (SEQ ID NO 82; 25 pmol/ul), 1 ul Alcust seq 10r primer(SEQ ID NO 83; 25 pmol/ul). PCR conditions: 95° C. 10 min, (95° C. 30 sec, 55° C. 30 sec, 72° C. 1 min) 35 cycles, 72° C. 5 min. Reactions are analysed by agarose gel electrophoresis. Colonies showing the correct sized fragment are used to prepare small scale cultures from which DNA is prepared (using Qiagen Spin minipreps). DNA is sequence verified to check the orientation of the alcR. Clones were designated pUC Sally ustus AlcR (FIG. 7).

pUC Sally ustus AlcR and pVB6 are digested with FseI (37° C. 3 hrs). Following confirmation of digestion on a 1% gel, the pVB6 vector is purified (Geneclean spin, Bio101), phosphatased using SAP (37° C., 1 hr) and cleaned up (Geneclean spin, Bio101). The required band from pUC Sally ustus AlcR is purified from 1% agarose (Geneclean spin, Bio101). Ligations are then set up using 3 ul insert and 1 ul phophatased vector (T4 DNA ligase, 16° C., 16 hrs) and the DNA transformed into DH5α competent cells.

Colonies were analysed by PCR screening as described above, using primers having SEQ ID NOs 84 and 85. Colonies showing the correct sized fragment are used to prepare small scale cultures from which DNA is prepared (using Qiagen Spin minipreps). Analytical XbaI and XmaI (NEB) restriction digests are performed (37° C., 3 hrs) and analysed on 1% agarose gel. Clones showing correct banding pattern are sequence verified and designated pVB ust (FIG. 8).

DNA is transformed into Agrobacterium strain MOG301 using electroporation (50 ul competent cells, 1 ul DNA in 0.2 cm cuvette, electroporated using Biorad Gene pulser at 25 uF, 200 ohms, 2.5 kV), following addition of 1 ml LB media, cells are incubated at 28° C. for 1 hr. 25 ul is spread onto LB+Kan+Rif plates and incubated at 28° C. for 2 days. Colonies are analysed by PCR using NPT2-2 and p35S-3 primers. A clone showing the correct band is used for further work (see example 6.3 below)

6.2.2 Expression Vector for the Expression of A. fumigatus alcR Sequence

The A. fumigatus alcR is amplified by PCR from pCR2.1 using Sally 21 (SEQ ID NO 86) and Sally 22 (SEQ ID NO 87), thus adding SalI sites to both 3′ and 5′ ends: 1 ul pCR2.1 fumigatus AlcR, 1 ul each primer at 100 ng/ul, 1 PCR bead, made up to 25 ul with H₂O. PCR conditions are: 95° C. 5 min, (95° C. 1 min, 55° C. 1 min, 72° C. 2.5 min)×40 cycles, 72° C. 10 min. This PCR product is then cloned into pGEM-Teasy following the manufacturers protocol. The ligated product is transformed into TOP10 cells

Colonies are analysed by restriction digestion with SalI. Clones exhibiting the desired restriction pattern are designated M192 (FIG. 9). pUC Sally nidulans II is digested with Sail (37° C., 3 hrs) and the required band was purified from 1% agarose using Bio10t Geneclean spin kit. This fragment is phosphatased using SAP (37° C., 1 hr) and the DNA cleaned using the Geneclean spin kit (Bio101). The M192 vector containing A. fumigatus alcR is partially digested with SalI using 1/2[enzyme] (37° C., 15 min) and the alcR fragment is purified from 1% agarose (Bio101 Geneclean spin).

Ligations are set up (6 ul alcR fragment and 1 ul phosphatased vector, T4 DNA ligase, 16 hrs, 16° C.) and the DNA transformed into DH5α competent cells.

Colonies are analysed by PCR using primers having SEQ ID NOs 82 and 88.: those showing the correct sized fragment are used to prepare small scale cultures from which DNA is prepared (using. Qiagen Spin minipreps). XmaI restriction digests are performed to determine the orientation of the alcR (3 ul DNA+1 ul buffer+1 ul XmaI+5 ul H₂O, 37° C., 2 hrs). A clone showing the correct banding pattern (called pUC Sally fumigatus AlcR*) was sequenced. Sequence analysis shows an error in the sequence at position 1615 bp (T to C). This error is corrected by digesting pUC Sally fumigatus AlcR* and pCR2.1 fumigatus AlcR (the original vector from cloning the alcR from genomic DNA) with BglII and SacI (37° C., 3 hrs). The large, vector fragment from pUC Sally fumigatus AlcR and the small fragment containing the correct sequence from pCR2.1 fumigatus AlcR are purified from 1% agarose (geneclean spin, Bio101). Ligations are then set up using 3 ul insert and 1 ul vector (T4 DNA ligase, 16 hrs, 16° C.) and the DNA was transformed into DH5α competent cells. Colonies are used to prepare small scale cultures from which DNA is prepared (using Qiagen Spin minipreps). The resulting DNA is sequenced to check whether the error has been corrected. Clones with corrected DNA sequence were designated pUC Sally fumigatus AlcR (FIG. 10).

pUC Sally fumigatus AlcR and pVB6 are digested with FseI (37° C. 3 hrs). Following confirmation of digestion on 1% gel, the pVB6 vector is purified (Geneclean spin, Bio101), phosphatased (17 ul DNA+2 ul phosphate buffer+1 ul SAP at 37° C. for 1 hr) and cleaned up (Geneclean spin, Bio101). The required band from pUC Sally ustus AlcR is purified from 1% agarose (Geneclean spin, Bio101). Ligations are then performed and the DNA transformed into DH5α competent cells, as described previously.

Colonies were screened by PCR using NPT2-2 and p35 S-3 primers (SEQ ID NOs 84 and 85 respectively) as described previously. Colonies showing the correct sized fragment are used to prepare small scale cultures from which DNA is prepared (using Qiagen Spin minipreps). Analytical XmaI (NEB) restriction digests are performed and clones showing correct banding pattern are sequence verified and designated pVB fum FIG. 11).

DNA is transformed into Agrobacterium strain MOG301 using electroporation as described above. Colonies are analysed by PCR screening and those clones showing the correct sized band are used for all further work (see example 6.3 below).

6.2.3 Expression Vector for the Expression of A. versicolor alcR Sequence

The A. versicolor alcR is amplified by PCR from pCR2.1 using Sally 12 (SEQ ID NO 89) and Sally 13 (SEQ ID NO 90) adding SalI sites to both 3′ and 5′ ends: 1 ul pCR2.1 versicolor AlcR, 1 ul each primer at 10 pmol/ul, 4 ul buffer, 4 ul 2.5 mM dNTPmix, 2 ul pfu, 27 ul H₂O. PCR conditions are: 95° C. 2 min, (95° C. 30 sec, 55° C. 30 sec, 72° C. 5 min)×30 cycles, 72° C. 10 min. This PCR product is then cloned into pCR2.1 TOPO (Invitrogen) following the manufacturers protocol. The ligated TOPO product is transformed into TOP10 cells.

Colonies are analysed by PCR (using M13for primer, SEQ ID NO 91 and M13 rev primer, SEQ ID NO 92) as described previously. Colonies showing the correct sized fragment are used to prepare small scale cultures from which DNA is prepared (using Qiagen Spin minipreps). Analytical EcoRI (NEB) restriction are performed and clones showing correct banding pattern are sequence verified and designated pCR2.1 Sally versicolor AlcR.

The pUC Kelly vector is digested with SalI (37° C., 3 hrs). Following confirmation of digestion on 1% gel, the vector is purified (Geneclean spin, Bio101), phosphatased with SAP (37° C., 1 hr) and cleaned up (Geneclean spin, Bio101). pCR2.1 Sally versicolor AlcR is partially digested with SalI (1/2[enzyme] at 37° C., 15 min) and the alcR fragment purified from 1% agarose (Bio101 Geneclean spin).

Ligations are set up using 3 ul AlcR fragment and 1 ul phosphatased vector (T4 DNA ligase, 16 hrs, 16° C.) and the DNA was transformed into TOP10 competent cells. Colonies are screened by PCR (using Alcvers seq2 primer, SEQ ID NO 93 and Alcvers seq1r primer, SEQ ID NO 94) as described previously, and those exhibiting a band of the correct size were used to seed small scale cultures from which DNA is prepared (using is Qiagen Spin minipreps). Analytical SmaI (NEB) restriction digests are performed and clones showing correct banding pattern are sequence verified and designated pUC Kelly versicolor AlcR (FIG. 12).

pUC Kelly versicolor AlcR and pVB6 are digested with FseI (37° C., 3 hrs). Following confirmation of digestion on a 1% gel, the pVB6 vector is purified (Geneclean spin, Bio101) and phosphatased with SAP (37° C., 1 hr) and cleaned up (Geneclean spin, Bio101). The required band from pUC Kelly versicolor AlcR is purified from 1% agarose (Geneclean spin, Bio101). Ligations are set up using 3 ul insert and 1 ul phophatased vector (T4 DNA ligase, 16° C. 16 hrs) and the DNA transformed into DH5α competent cells.

Colonies are screened by PCR using NPT2-2 and p35 S-3 primers (SEQ ID NOs 84 and 85 respectively) as described previously. Colonies showing the correct sized fragment are used to prepare small scale cultures from which DNA is prepared (using Qiagen Spin minipreps). Analytical SmaI (NEB) restriction digests are performed on and those clones showing the correct banding pattern are sequence verified and designated pVB ver (FIG. 13).

DNA is transformed into Agrobacterium strain MOG301 using electroporation as described above. Colonies are analysed by PCR screening and those clones showing the correct sized band are used for all further work (see example 6.3 below).

6.2.4 Expression Vector for the Expression of A. flavus r alcR Sequence The AlcR

The A. flavus alcR is amplified by PCR from pCR2.1 using “knpflav for” (SEQ ID NO 96) and “flavkpnI rev-2” (SEQ ID NO 97) thus adding KpnI sites to both 3′ and 5′ ends: 1 ul pCR2.1 versicolor AlcR, 1 ul each primer (10 pmol/ul), 4 ul buffer, 4 ul 2.5 mM dNTP mix, 2 ul pfu,27 ul H₂O. PCR conditions are: 95° C. 2 min, (95° C. 30 sec, 55° C. 30 sec, 72 CC 5 min)×30 cycles, 72° C. 10 min. This PCR is then cloned into pCR2.1TOPO (Invitrogen) following the manufacturers protocol. The ligated TOPO product is transformed into TOP10 cells.

Colonies are analysed by PCR (using M13 for primer, SEQ ID NO 91 and M13 rev primer, SEQ ID NO 92) as described previously. Colonies showing the correct sized fragment are used to prepare small scale cultures from which DNA is prepared (using Qiagen Spin minipreps). Analytical EcoRI (NEB) restriction digests are performed and clones showing the correct banding pattern are sequence verified and designated pCR2.1 Kelly flavus AlcR.

The pUC Kelly vector (see below for details) is digested with KpnI (37° C., 3 hrs). Following confirmation of digestion on 1% gel, the vector is purified (Geneclean spin, Bio101), phosphatased with SAP (37° C., 1 hr) and cleaned up (Geneclean spin, Bio101). pCR2.1 Kelly flavus AlcR is digested with KpnI (37° C., 3 hrs) and the alcR fragment purified from 1% agarose (Bio101 Geneclean spin).

Ligations are set up using 3 ul AlcR fragment and 1 ul phosphatased vector (T4 DNA ligase, 16 hrs, 16° C.) and the DNA transformed into TOP10 competent cells. Colonies are used to prepare small scale cultures from which DNA is prepared (using Qiagen Spin minipreps). SmaI restriction digests are performed and a clone showing the correct band is sequence verified and designated pUC Kelly flavus AlcR (FIG. 14).

pVB6 is digested with FseI (37° C., 3 hrs). Following confirmation of digestion on 1% gel, the vector is purified (Geneclean spin, Bio101) and phosphatased with SAP (37° C., 1 hr) and cleaned up (Geneclean spin, Bio101). pUC Kelly flavus AlcR is partially digested with FseI (1/2[enzyme], 37° C., 15 min) and the required band is purified from 1% agarose (Geneclean spin, Bio101). Ligations are set up using 3 ul insert and 1 ul phophatased vector (T4 DNA ligase, 16° C. 16 hrs) and the DNA transformed into DH5α competent cells.

Colonies are analysed by PCR (using M13 for primer, SEQ ID NO 91 and M13 rev primer, SEQ ID NO 92) as described previously. Colonies showing the correct sized fragment are used to prepare small scale cultures from which DNA is prepared (using Qiagen Spin minipreps). Analytical XbaI restriction digests are performed and a clone showing the correct banding pattern is sequence verified and designated pVB flav (FIG. 15).

DNA is transformed into Agrobacterium strain MOG301 using electroporation as described above. Colonies are analysed by PCR screening and those clones showing the correct sized band are used for all further work (see example 6.3 below).

6.3 Agrobacterium Mediated Transformation of Arabidopsis Plants.

Arabidopsis Columbia plants are grown from seed to flowering. The primary flower bolt is removed once flowers open to encourage growth of secondary flower spikes. Plants are used approx. 12-14 weeks after sowing.

Two 10 ml cultures of Agrobacterium (MOG301 containing the required construct) are established in LB+Kan+Rif media and grown at 28° C., 230 rpm for 24 hrs. These are then transferred to two conical flasks containing 500 ml LB+Kan+Rif and grown at 28° C., 230 rpm for 24 hrs. Cells are harvested by centrifuging at 4000 rpm for 20 min and resuspended in 1 litre of infiltration media (50 g/l sucrose, 4.4 g/l MS salts, 1 ml/l B5 vitamins, 0.5 g/l MES, 0.044 uMol BAP, 200 μl/l Silwet L-77).

Just before transformation all open flowers and partially open buds are removed from the plants with tweezers. The infiltration media containing the resuspended bacteria is dispensed into single magenta vessels and placed in a vacuum chamber. Prepared plants are then turned upside down and placed in the magenta vessels. A vacuum of 850 mbar is then applied for 10 minutes after which the vacuum is released slowly. Plants are blotted briefly on tissue paper and placed on their side in a sealed plastic bag for 24 hours. Plants are removed from the plastic bag and placed upright in mesh propagators to mature and set seed.

Three weeks after the transformation plants are placed on paper. The plants are no longer watered and left to dry down fully for 2 weeks. After that the plants are cut from the pots and placed in a brown paper bag.

To isolate the seed the paper bag containing the plants is rubbed to open the dried seedpods. Tapping the dried plant material through a double layer of mesh cloth separates the chaff and seed.

Fine sand is added to 3000 seeds and sown onto trays (9×14 inches) filled with wet 50:50 JI no.3/peat compost. The trays are placed in the fridge for 3-4 days and then placed in the growth room (16 hour photoperiod, 20° C. day, 16° C. night). 10 days later the germinated seedlings are ready for selection.

Seedlings are sprayed with a 0.1% Triton X-100 solution with Kanamycin at concentrations ranging from 100 to 500 mg/l using a 1.5 litre spray bottle. The Kanamycin spray is supplied in sufficient quantity to just wet the leaves. The plants are sprayed for two days with 100 mg/l Kanamycin, followed by 2 days with 200 mg/l Kanamycin, followed by 1 day with 500 mg/l Kanamycin. After each spray, trays are covered with a plastic dome in order to prevent excessive dehydration. Transgenics can be distinguished from escapes after one week on the basis of colour difference; dark green are transgenic, light green/bleached are untransformed.

Identified transgenics are transferred from the trays into 1-inch seedling trays containing Sinclair potting compost. Plants are left for one week in the growth cabinet (Day length of 10 hours, temperature of 20° C. day and 18° C. night, relative humidity of 65-75% and light levels of approximately 160 μMol) in order become established.

### EXAMPLE 7

### Assessment of Activity of AlcR Orthologues in Plant

7.1 Agrobacterium Infiltration of Tobacco Leaves—A Transient Expression System

Agrobacterium transformed with a construct of interest is grown overnight in the presence of selection media at 28° C. A 1/10 dilution is made of the overnight culture and grown freshly during the day. The OD of the culture should reach A₆₀₀ 0.6. The culture is spun for 5 minutes at 4000×g and then resuspended in 10 mM MgSO₄ and incubated in ice for 60 minutes. A 1 ml syringe is used to inject in the underside of the leaf (no needle and gentle pressure applied y finger on the opposite side of the leaf). Penetration of the liquid is readily observed as the leaf becomes more translucid. The Plant is left in the glass house for three day upon which it is watered with a 5% ethanol solution. Two days later the tissue is collected and subjected to GUS histochemical staining.

7.1.1 β-glucuronidase (GUS) Histochemical Staining

12.5 mg of X-Gluc (Sigma) were dissolved in 25 ml of 100 mM Phosphate buffer (pH 7.0) supplemented with 0.01% Triton X-100 and DMSO. The leaf material in submerged in the buffer and the buffer is vacuum infiltrated into the leaf. Material is then incubated at 37° C. for S to 24 hours. The buffer is discarded and the material is distained using several washes of 100% ethanol over a period of 48 hours. Photographs of the material were taken.

7.2 Transgenic Plant Analysis

Primary transformants coming through antibiotic selection are assayed for the presence of the transformation construct via PCR. This assayd for the gus gene and the alcR gene. Plants that are found to be positive have tissue taken from them and are induced with a 2% (v/v) Ethanol root drench (20 mls in a 2′ pot). The plants are left in the glasshouse under normal watering and light regimes. 3 days after treatment 3 leaves are collected from each plant and each leaf is assayed independently. GUS and protein assays are carried out.

7.2.1 GUS Assays

Plant material is harvested and are resuspended in 300 ul of GUS extraction buffer (Jefferson et al., 1987 “GUS Fusions: O-glucuronidase as a sensitive and versatile gene fusion marker in higher plants” EMBO J. 6, 3901-3907) to prepare β-glucuronidase extracts. The plant material is homogenised for 1 minute and then are centrifuged (13000 rpm for 2 minutes). The supernatant is transferred to a fresh eppendorf tube. 20 ul of the extract are used in the GUS assays. Fluorometric assays for GUS activity are performed using 4-methylumbelliferyl-D-glucuronide (Sigma) as a substrate and fluorecence is measured in a Perkin-Elmer LS-35 fluorometer (Jefferson et al., supra). Protein concentrations of the tissue homogenates are determined by the Bio-Rad protein assay (Bradford, 1976 “A rapid and sensitive method for the quantification of microgram quantities of protein utilising the principle of protein-dye binding” Anal. Biochem. 72,248-254)

7.3 Results

Alignment of the amino acid sequences of the AlcR orthologues allowed identification of amino acid motifs 1 to 17 (SEQ ID NOs 104-120; FIG. 16). In order to test for inducible activity in plants, tobacco plants were infiltrated as described above. Two plants were infiltrated, one was treated with 5% v/v ethanol whilst a fourth was only watered. The plants were left in the glasshouse for a total of 5 days (treatment carried out at day 3) and the induced and un-induced tissues were harvested and histochemically stained. Plant material that is infiltrated with an Agrobacterium stain containing the pVB6 ver (FIG. 13), pVb ust (FIG. 8) or pVB fum (FIG. 11) respectively, shows blue staining when induced with ethanol, denoting the presence of the reporter gene un-induced plant material does not exhibit colouration, indicating the lack of reporter gene activity.

Sequences

All nucleotide sequences are described in the 5′ to 3′ orientation, using the standard single letter code. All amino acid sequences are described in the N-terminal to C-terminal orientation using the standard single letter code, as described above.

SEQ ID NO 1. Degenerate oligonucleotide forward direction Alcla2:
TGYGAYCCLTGYCGIAARGGIAAA
SEQ ID NO 2. Degenerate oligonucleotide forward direction Alclb2:
TGYGAYCCITGYCGIAARGGIAAG
SEQ ID NO 3. Degenerate oligonucleotide forward direction Alclc2:
TGYGAYCCLTGYCGRAARGGIAAA
SEQ ID NO 4. Degenerate oligonucleotide forward direction Alcld2:
TGYGAYCCITGYCGRAARGGIAAG
SEQ ID NO 5. Degenerate oligonucleotide reverse complement Alcrev1a:
CCTICGYTTRCARTTIGARCA
SEQ ID NO 6. Degenerate oligonucleotide reverse complement Alcrev1b:
CCTICGYTTRCARTTRCTRCA
SEQ ID NO 7. Degenerate oligonucleotide reverse complement Alcrev1c:
CCTYCGYTTRCARTTIGARCA
SEQ ID NO 8. Degenerate oligonucleotide reverse complement Alcrev1d:
CCTYCGYTTRCARTTRCTRCA
SEQ ID NO 9. Degenerate PCR DNA fragment from Asperigillus ustus genomic DNA
with identity to alcR:
GAATTCGCCCTTTGTGAYCCGTGYAGRAAAGGGAGRCGAGGGTGTGATGCG
CCTGTGAGTTGACTCGTGCCTACCTGCCTCGCTTCAAAGGCAGAATCAGGCC
ATACGCGCCCTATGCCTGCGAAGAATCCGGAATTCTCTAACGCCACTCCAG
GAAAATCGAAGTGGAGATGGATACACCTGCTCCAACTGYAAGMGVAGGAA
GGGCGAATTC
SEQ ID NO 10. Degenerate PCR DNA fragment from Aspergillus fumigatus genomic
DNA with identity to alcR:
GAATTCGCCCTTTGTGATCCGTGTCGGAAGGGGAAGCGGGCGTGCGATGCG
CCTGCTCGTAGAGACCGGCACGCGGACGCCGGCAGCCGAAGGGTGCTAGCA
GAGAGCAACCTCAACATCCCGTGCTCCAACTGCAARCGCAGGAAGGGCGAA
TTC
SEQ ID NO 11. Degenerate PCR DNA fragment from Aspergillus versicolor genomic
DNA:
GAATTCGCCCTTTGTGATCCGTGTCGGAAGGGGAAGCGAGGGTGTGATGCG
CCTGTTXGTTGACACCGGCAAAGATCTTAAACGCGAATCCGAAAGTGCCAC
TCGAGAAAATGGCAACTGGATACTCGTCCAACTGCAAGCGCAGGAAGG
GCGAATTCX
SEQ ID NO 12. Degenerate oligonucleotide reverse complement Alc7001a:
ATHTAYCAYGAYTCNATGGARAAT
SEQ ID NO 13. Degenerate oligonucleotide reverse complement Alc7001b:
ATHTAYCAYGAYTCNATGGARAAC
SEQ ID NO 14. Degenerate oligonucleotide reverse complement Alc7001c:
ATHTAYCAYGAYAGYATGGARAAT
SEQ ID NO 15. Degenerate oligonucleotide reverse complement Alc7001d:
ATHTAYCAYGAYAGYATGGARAAC
SEQ ID NO 16. Degenerate PCR DNA fragment from Aspergillus flavus genomic DNA
with identity to alcR:
GAATTCGCCCTTTGTCATCCGTGYCGGAAAGGGAAGAGAGCATGCGATGCC
CTCCTGGCTGACGAGCTTGAACGGAATTCCAACACTGCTGCTCGACAAGCGT
ACAATCACGCGTGCTCCAACTGCAAAAAATACAAAAGAAAATGCACGTTCG
ACTGGCTCTTGAGTCACAAGGAATCCCGGCATGCTCATAGCAAGAGAGCCA
GAAATATCGCGATCGCCCCTCGCGGCAGGTGAACGATTGTTCCGCTCATTC
CTCTCAACAAACCCCACTGGGCGCAATCCTACAGAGCTCCCTCTGCAAAAC
ATCGAGGATTGCGAATGGCCAACGTCTGTTAGGGACCCGCTTTTGCCGTTCC
CACAAGACGAGGAACTAGATGCGGACTGGTTAACTTGGGGATGCCTCAACG
ACGCAGTGTCCATCTCTCCTCTAAGCGCCGACATGACTCTCAATGGGGATAG
GCACGTCAATCGTAACCAGACACCACAAATGAGTACTCAATGGAACTCTGT
CGGGGCCGGCCAGGCATGGCAAAGTATCGGTCAAACTTCACTGCTCGACAC
GATGAACAGTTCTATAACTTCGTCGCAATTCAAGGATACACCCGACTATCGA
TCATTGAGACATGGGATATCAGTTCTGGGCTCCCGCTTCACGGTCTTCCAC
CTACCGAAGGACGTGGTGTGTCQATGCCAACAAACACTACACTGTGTGTGG
GCTCAAACCAATTAGCACACAATTATGCGCACTCCATGATGACGCGCAACC
TAATCACATMTACCACGACAGCATGGAAAATAAGGGCGAATTC
SEQ ID NO 17. Aspergillus fumigatus genomic DNA fragment encoding the full open
reading frame of the A. fumigatus alcR:
GAATTCGCCCTTATGGAGGCTCATCGTCGACGCCAGCACCACAGCTGCGAT
CCATGTCGGAAGGGGAAGCGGGCGTGCGATGCGCCTGCTCGTAGAGACCGG
CACGCGGACGCCGGCAGCCGAAGGGTGCTAGCAGAGAGCAACCTCAACATC
CCGTGCTCGAACTGCAGGAAATACAATCGAGAATGCACGTTCAACTGGTTA
GTCGAGAACCGCGCCGCCGCACGGGCGGGTCGAAAGCAGAAGAgCCGTAAT
GTGAGCAACTTGCCTCGAGCGGACGACGTGAGTTCGAGTCGCTCGGGAACC
GACCTGCTGGACGATCTGCGGTACTCCTCGTCGTGGCTATCCAACAGTCCTG
GGAATGGGGTGTCGTCGAACGGTFCGACGGAGGACCAGCCCGGGACGTGGT
CGATGCCGTCGAATGCCGTCTCGATACCGGTGAGAAGCAAGGAGTCGGAAC
TCGATCCGTTCAGTGTCATGCTGTGGAATGCAAATACAGCACACGTACCGCC
GAGCAATGCGGAAACGGCGGGCTCGGGCTGAGGACACTTGTTCGAGTCTGGA
CTACTACCAGCAGAGCTTGTCCAGTTCGGGACCGCACTCGCTCGACGAGAC
GCTAGATCTACTTCAACAGTTCGATGATTCGAGTCCAGGATTGAGTAGCTCG
TATTACTCTTCGCCACCTGGCTTTGTGATTCCGGAAGGTAGTGACGGTCTAC
CGACATTCCCGGAGACAGTCTCTATCCCTCCGGGAACAAAGATAGTCTATT
TGTTCTTTCCGATAACATCTCAGACAGCTATGCCCGCTCGATGATGACACAG
AATCTTATCCGCATATACCATGACAGCATGGAGAATGCGTTGTCCTGCTGGC
TCACGGAGCAAAACTGTCCCTACAACACGGCAGTCCCGTACACCTCACCGA
GCGGGCTCGCCAGTAAGGCACAAGCGGCATGGGCCCCGAACTGGACGAACC
GGATCTGTACTCGGGTCTGTCGGCTCGATCGAGCGTATGCATCCGTCCGTGG
GCGAAACCTCAGCGCCGCAGAAGAGAAAATGGCATCGAGAGCGCTCCACA
CCGCCATCATGGCGTTCGCCTCGCAGTGGGCGCAGAAGATGCCCAGAAGCA
ATGGCTTTTCTCTTACCTCGCCCGTCGCGCAGCACGAGCGTGTCATCCGGGA
GAATGTGTGGAACCAGGCGCGGCGTGCTCTGGAGAATGCAGCGGGTATCCC
TTCGTTCCGGGTTGCGTTTGCGAACATCATCTTCTCCATCGGACAGCGTCCG
CTCAATGTCGATGAGGACATGGAGCTGCATGAGTTGCTGGAGAATGACAGC
GCGCCGTTGTTCATGGAGGCGGCGGTGCGACAGCTGTTTTCAATCCGATATA
AACTGACCCGTCTCGAGCGGCAGAAGCCAAAGTCGCGAAGTTCGCCAGAGC
AGAGCAAGATCGATCTCGCCAGTATGGATATGCCGTCGCCACAGACGGATG
CGTTCTATGCCGACCCGGAGCACCAGGAAACCGTCAACCTCCTGTTCTGGCT
GGTGGTCATGTTCGACACCCTGCAGGCGGCCATGTATCAGCGTCCCCTCGCC
ATCTCCGACGAGGACAGCCAGATCACGTCCGTGTCACCGGCGGTCTCCAAC
GCCAAACCCGACAGCAGCGTCGACCTCGACGGCTGGAACATCACGTACTCC
CGCGCCCTGAAAGAGAAACAAGACCTCTGGGGCGACTTCTTCCTCCACAAA
CGCGCCGCACGCCAGGGCGCGAACCCACCCCGCTGGCCCTGCTCCTACGAA
GAAGCCGCCGAGATCCTCTCCGACGCCAGCCCCGTCAAAGTCCTCCTCTTCC
GACAAGTCACCCGCCTCCAGACCCTCGTCTACCGCGGCGCCAGTCCCGACC
GCCTGGAGGAGATCATCCAAAAGACGCTGCGCATCTACCAACACTGGAACA
CCACCTACAAGCAATTCTTCCAGAGCTGCAACGCAAACCACGACGATCTGC
CCCCGCGCATCCAGTCGTGGTACGTCATCGTCGCAGGGCACTGGCATCTCGC
CGCCATGCTGCTCGCCGACACCGTCAAGGGCATCGACGAGGGCCACCTCGG
CCTGGACAGCCGGCGCGAAGCCCGCACCGCAATCGACTTCGTCGCCACCCT
CCGGCGGGACAACGCGCTGGCCGTCGGGGCCATCGCTCAGCGCTCCCTGCA
GGGGCGGGACTCCCTGGCCAACCGCATCCAGTTCTACCACGACGCCGTGAA
CGAGGCCGCGTTTCTGACGGAGCCGTGGACGCTCGTCCTGATTCGCTGTTTC
GCCAAGGCGGCGTATATTCTGCTAGACGACATCACGCCGCAGTCGCACGGC
GCGCGGCCGGACGACCCGTCCGAGTACGCCCGOCGGAACTGCGAGTTCTGT
ATCTCGCGCTGTGGTGTCTGGGGACGAAATCGGACATGGCGTITGTGUCTG
CGCGCTCGTTGTCGAAGCTGCTGGATACGCGACTAGGGAAAGGTGTCGATC
AGTTCTGTTCCGTAGGGGAGGGTGCTCGGATTCCGTCCATGCCGCTTTTTGA
TGAACGGGGATCGGGCGAGTTGGGCAGTGTCGGGATCTCGGTGTAGTTAAG
GGCGAATTC
SEQ ID NO 18. ORF predicted from genomic DNA sequence derived from A.
fumigatus:
MEARRRRQHHSCDPCRKGKRACDAPARRDRHADAGSRRVLAIESNLNWCSNC
RXYNRBCTFNWLVENRAAARAGRICQKSRNVSNLPRADDVSSSRSGTDLLDDL
RYSSSWLSNSPGNGVSSNGSTEDQPGTWSMPSNAVSIFLRSKESELDPFSVMLW
NANTAHVIPPSNAETAGSAEDTCSSLDYYQQSLSSSGPHSLDETLDLLQQFDDSSP
GLSSSYYSSPPGFVIPEGSDGLPTFPADSLYPSGNKDSLFVLSDNISDSYARSMMT
QNLIRIYHDSMENALSCWLTEQNCPYNTAVPYTSPSGLASKAQAAWAPNWTNR
ICTRVCRLDRAYASVRGRNLSAAEEKMASRALHTAIMAFASQWAQKMPRSNG
FSLTSPVAQHBRVIRENLWNQARRALENAAGIPSFRVAFANIIPSIGQRPLNVDED
MUELLENDSAPLFMEAAVRQLFSIRYKLTRLERQKPKSRSSPEQSKIDLASMD
MPSPQTDAFYADPEHQETVNLLFWLVVMFDTLQAAMYQRPLAISDEDSQITSVS
PAVSNAKPDSSVDLDGWNITYSRALKEKQDLWGDFFLHKRAARQGANPPRWP
CSYEEAAEILSDASPVKVLLFRQVTRLQTLVYRGASPDRLEEIIQKTLRIYQHWN
TTYKQFFQSCNANHDDLPPRIQSWYVIVAGHWHLAAMLLADTVKGIDEGHLGL
DSRREARTAIDFVATLRRDNALAVGAIAQRSLQGRDSLANRIQFYHDAVNEAAF
LTEPWTLVCFAKAAYILLDDIPQSHGARPDDPSEYARRNCEFCISALWCLG
TKSDMAFVAARSLSDTRLGKGVDQFCSVGEGARIPSMPLFDERGSGELGSV
GISV
SEQ ID NO 19. AF Alc gen1: TGCGATUCGCCTGCTCGTAGAGACCG
SEQ ID NO 20. AFAlcgen1:AGGGTGCTAGCAGAGAGCAACCTCAAC
SEQ ID NO 21. Alcfum walk3a: CGTGCTCTGGAGAATGCAGCGGGTATC
SEQ ID NO 22. Alcfum walk3: GCTGCATGAGTTGCAGGAGAATGACAG
SEQ ID NO 23. Sense oligonucleotide (Fum for) for amplification of the whole ORF of
the alcR orthologue: ATGGAGGCTCATCGTCGACGCCAG
SEQ ID NO 24. Antisense oligonucleotide (Fum rev) for amplification of the whole
ORF of the alcR orthologue: AACTACACCGAGATCCCGACACTG
SEQ ID NO 25. Genomic 5′ sequence to the ORF from the alcR orthologue gene of A.
fumigatus.
GAATTCGCCCTTACTATAGGGCACGCGTGGTCGACGGCCCGGGCTGGTATCC
TTGCTACACTGCTAAACAACGGCACCTCACCCATCACCGGCAAGCGAATCC
TCGAGACAACCACAGTCGACGAGATGTTCCGCAATCAGATCCCCAACCTCC
CCAATTTTGCCGCACAAGGCATCCCTCGTFCGAAGCCTGACCTCACCAATGA
AATAGCTCATCTGTACCCATCGCCGACACCTCAGGGGTGGGGCCTCACCTTT
ATGCTGACGGGAGGTCCACTGGACGGTCTGAAGGGACGGCGCACTGGGCA
GGACTTGCGAACCTCTGGTGGTGGTGCGATAGGGAGAAAGGGGTCGCAGGG
ATGATTGTACTCAACTCTTGCCCYYYGCTGATCCCCAAGTTTGGAGCCTTTG
GCTGGATGTGGGAGTCTGCCGTCTACCGTGGCCTGGCTCAGGATTAGACTCTG
CCGTATCAMTGCTCCTCCTGAGATATTTCTATATGATTGGACTAGTTTCCAT
CAGTCAGTCCGTTCTTTTGTTTTTTTTTTTTTTTTTTTATAGACTTTGAACTC
AATACCTCCGGTCATCCGAAGCTGGCRTGCTGAAKCGCTSAAKKGGYRTWC
MYCASKRGRTRTGWCMSYTYGCMAAMAMRAAGYSKWRAGMKCWWCCGC
CAYCGCAGTCCAACCACCCAACCAGCGCATCACTCGGACGCAAACAGACTC
AACGACTCGTCCTAGTGCGCCGACAATCCAGGCAGCGATAAACCAGTCAGG
TCTCGTGAACTCCCTCCCAGAACCACCAGACTTCGCGAATCCCCAGACCCCG
CATCGTGCTCTTGGCTCGGAGCTTCAARACCCGCCTAGCCATGAGGTGGTCT
CTCTCACACTGTATCCCCCCTCCCCCCATATCTGTCTCCACAATAGCCATCAC
CCGGTAATAGCCGAATTTGTATGCCGGCATACCGTAGCGCTTGGAGACAAC
TGTCAGTGCCACGATG
SEQ ID NO 26. Alcfum walkup1: GTGAGGTTGCTCTCTGCTAGCACCCT3′
SEQ ID NO 27. Alcfum walkup2: CGGTCTCTACGAGCAGGCGCATCGCA3′
SEQ ID NO 28. Alcust walk1: CGCITCAAAGGCAGAATCAGGCCATAC
SEQ ID NO 29.Alcust walk2: ATCCGGAATTCTCTAACGCCACTCCAG
SEQ ID NO 30. Alcust walk3: ATGCCGACCCGATGAGCGCAATGCTAC
SEQ ID NO 31. Alcust walk4: ATACGCGGAAGGGCACTGAGCGTAGAC
SEQ ID NO 32. Alcust walk5: CTACAACACTCCACAGGGATCCCGTC
SEQ ID NO 33. Alcust walk6: CACAGAGTCCGCTGGACGAGAATCGAC
SEQ ID NO 34. Alcust walkup1: CTGGAGTGGCGTTAGAGAATTCCGGAT
SEQ ID NO 35. Alcust walkup2: CTATGGCCTGMTCTGCCTTTGAAGCG
SEQ ID NO 36. Sense oligonucleotide (Alcust for) for the intact ORF amplification
from genomic DNA and first strand cDNA: CTCGAATGAAGATGGGAGACTC
SEQ ID NO 37. Antisense oligonucleotide (Alcust rev) for the intact ORF amplification
from genomic DNA and first strand cDNA: TTACACAAGGATATCCGCTGAC
SEQ ID NO 38. Asperigillus ustus genomic DNA fragment encoding the full open
reading frame of the A. ustus alcR orthologue:
GAATTCGCCCTTCTCGAATGAAGATGGGAGACTCCCGTCGCCGCCAGAATC
ATAGCTGCGATCCGTGTCGCAAGGGGAAACGAGGGTGTGATGCGCCTGTGA
GTTGACTCGTGCCTACCTGCCTCGCTTCAAAGGCAGAATCAGGCCATACGCG
CCCTATGCCTGCGAAGAATCCGGAATTCTCTAACGCCACTCCAGGAAAATC
GAAGTGGAGATGGATACACCTGCTCGAATTGCAAGCGGTGGAAGAAGAAAT
GCACATTCAATTTCGTCTCGTCCAGGCGCGCAGATTCCCGCGTCGTCGGTGC
CAATGCCGGGTCAAAAGCGAAGTCCACCTCTACCCCTGCTGTCTCTACCGCT
GCATCGGTAGCCACTFCTGCAGCTGCCCCTCCCACTCCCGATAGTGGCGACA
TCCCTGCCATGCTAAACACGGGTATGGACATGGGCACGAATGAGTACGATG
CTCTCCTTCATGACGGTTTGCGGTCGTCACACCTTGACCCTACGAGGCTTGG
GGATATGTTTGCTTTTACCTCGCCGTCTAGTTTCACGGCGGAGGCTTTGCAT
GCGCAGAGTGCTGTTGGCACAGAAGCCATCGCGTGGGATTCAGGGATTCCA
ACAGACTGGTCTATCCCTTCGATGCCTCGGTCGGAAAAGTCGTTCACTCCGC
TTGAGAGTCAGGCGGTCTTTCTTGCACAGGAGGATTCGAACCAGTTTGACGT
TATTCAGGAGTTGGAAGATGGCTCATCCGACAACTTCACACCACCGGGGCG
GAAACGCGACGAGGATAAGCGACGGAAATTTCAATGGGAGTTATGCATCGC
TTCCGACAAAACAGCCAACCAGGTTGGCCGATCGACAATGACGCGCAATCT
ATGCGGATATATCACGATAGCATGGAGAATGCGCTCTCATGTTGGTTGACC
GAGCACAACTGTCCGTATGCCGACCCGATGAGCGCAATGCTACCTTTTAACC
AGAGGAAAGAATGGGGTCCCAGTTGGTCGAACAGGATGTGTATCCGGGTCT
GTCATTTAGATCGGGAATCATCCTCGATACGCGGAAGGGCACTGAGCGTAG
ACGAGGACCGGACGGCCGCGCGGGCGCTGCATCTCGCAATTGTCGCATTCG
CGTCACAGTGGACGCAGCATGCCCAAAGGGGGACAGGGCTTTCGGTTCCGA
CTGATATCGCTACGATGAACGGTCGATTCGAGAAAGAATATATGGAACGAGGC
GCGGCATGCTCTACAACACTCCACAGGGATCCCGTCTTTCCGGGTAATATTC
GCCAACATTATTTTCTCATTGACACAGAGTCCGCTGGACGAGAATCGACCTG
CGAAGCTAGGTCAGCTGTTGGAGAATGATGGTGCTCCCGTATTCCTAGAGA
ACGCCAATCGTCAGCTCTACACATTCCGACACAAGTTCGCGAGACTCCAAC
GAGAGGGTCCCCCGCCGTGGCTGGGCTGCGACGAGGTTCAATATCATCCA
CTCTCACTGACGTGCTGGAAGTTCCGACTCCTGAATTCCACAGGTCGATCC
AATTCTCGCGAATCAAGACCACCGAAGCACACTCAGCCTCCTCTTCTGGCTT
GGAATCATGTTCGACACCCTCAGTGCAGCCATGTACCAGCGCCCTTGTCG
TGCAGACGAAGATAGCCCAAATCGCCTCCGCCTCCCCGTCGGCCTCAACCA
ACCCCCGAGTCAACCTCAACTATTGGGAAATCCCAGACAGCAATCTCCCAG
CGAAAAACGACGTCTGGGGTGAATTTTTCCTTCAACCTGCCGCTCGCCAGGA
ACGGCCTCCGCACATCCCCAAATCCAACCAAAACAACCCCGTTGGCCGTG
TTCCTACGAAGAAGCCGCATCAGTCCTGTGCGAGGCAACACCGGTAAAAGT
CCTTCTCTACCGCCGSGTCACCCAACTCCAAACCCTTATCTACCGTGGCGCG
TCTCCCGCACGGCTTGAAGAAGTCATTCAAAGAACGCTTCTCGTCTACCACC
ATTGGACCTGCACATATCAATCATTTATGCTCGACTGTGTGGCAAACCACGA
GTCCCTTCCACACCGTATTCAGTCTTGGTATGTTATTCTTGATGGCCATTGGC
ACCTCTCCGCAATGCTTCTCGCCGATGTGCTAGAGTCCATCGACAGAAGCCA
CCTCGGACTCGAGTCGGAGCGCGAGTCCCGGATTGCAAGCAGATCTTATTGC
AACACTGCGAATCGACAATGCACTCGCAGTCGGTGCCTTGGCTAGGGCATC
GCTACACGGGGAGAATAGCATGATGCATCGACATTTCCATGACTCGTTGAA
CGAGGTCGCGTTCCTGGTTGAGCCGTGGACAGTCGTTTTGGTCCATTGTTTC
GCGAAGGCGGCGGCTATTTCGCTGGATTGTCTGGGTCAGGGACAGGGAGGT
GCTTTGGCAGAATGTTTTCGGCAGAATTGTGAATATTGTATTTGTGCGTTGA
AGTATTTGGGACGGAAATCGGACATGGCGTTTTGTGTTGCGGGCGGGTTGG
AGAAGGAGTTGCTTGAGAAAGCTGGGAGTATGCTGTCAGCGGATATCCTTG
TGTAAAAGGGCGAATTCC3′
SEQ ID NO 39. The Aspergillis ustus alcR promoter sequence; the translation start site
is at position 617:
TCGAGAATATACGAAGTCAAGACTGTCNGTGTACAGCTCAAGGC
TTAAGCAGAATGTTCTRAGAATATGGTYTGGTAGTTACATGTTCC
TAGTATGCTTTGATGATCTATTAGTCTCGTATACARGGAAGACAG
TATGATGTTAGTATGTATAAGAAGAGACTAGCTACGGTGATGTT
AAGAACTTACGTTCAAGATGCCGTATAATTTCCGAATACTCCAG
AGTATAACTCCGGATCGCCACCTCGTAGCTCTTAAATAAGCAAT
TCCAATTCTGCGAGTGCGACGTATCAACCAAGTGTCGGACTGCG
GGGGCGATCTCCGCCCCGAGAGTTCACGCTAGGCCCAGCACTGC
ATCGCCCCCACAGCGAGGTATRGKCCYCGCGTGCTATTGGCCTC
GTGCCCCGCGCACATCCTCACCGGAGTCGGAGGCAGCAGGAACT
TGGGGCTGGTCATGTGACAGCAAACCCCGCAGAGCCCAATGTT
GACTTTCCCCAGAATCTCGYCCAGCTGCGACAAATCCCGCCTTC
CCCAAGTCCCGTCTCGGAGATTGTCTCCACGTCCTTGTTAGAATA
ATCATCAATTCCGAATTGATACGTTACGTATCGTACCTCGAATG
SEQ ID NO 40. Alcvers walk1: AGGCGCTGTGATGCTCCGGTTTGTGGC
SEQ ID NO 41. Alcvers walk2: TGATATCAAATACTTCTTAGAGCAACCG
SEQ ID NO 42. Alcvers walkup1: CGGTTGCTCTAAGAAGTATTTGATATCA
SEQ ID NO 43. Alcvers walkup2: GCCACAAACCGGAGCATCACAGCGCCT
SEQ ID NO 44. Sense PCR primer (Alcvers for) for amplification of the whole ORF of
the A. versicolor alcR orthologue: GGTTGCTCGCCATGGATGAC.
SEQ ID NO 45. Antisense PCR primer (Alcvers rev) for the amplification of the whole
of the ORF of the A. versicolor alcR orthologue TTCATGGCATCCGGCEAAGC
SEQ ID NO 46. Aspergillus versicolor genomic DNA fragment encoding the full open
reading frame of the A. versicolor alcR orthologue:
AATTCGCCCTTGGTTGCTCGCCATGGATGACCCCCGCCGCCGCCAGTTTCAT
AGTTGTGACCCCTGTCGCAAGGGCAAGAGGCGCTGTGATGCTCCGGTTTGTG
GCCATCTCCCACTCTGCTTTTTATCATCGGCTAATTCTGATATCAAATACTTC
TTAGAGCAACCGGGAAAATGGTAACTTTGATTCTTGCACTAACTGCAAGCG
ATGGAAGAAAGAGTGCACATTTACCTGGCTCTCCTCGAAGCCAGCGAAGCG
TGCGGACCCCAAAGGACGAGCAAGACCGAAACCGGGCGTTTCGACTACTTC
TAGCAAACCTAGTGCTGCCAGCAACCCTAGCACTACTAGTAACCCTAGTAGT
GATAGCGGTGGGACACCTCCTGATCCAAGTCGCGTTGTCCCTTCCATGGTGG
GCTCCTATAATGCCCTCGTGGACGGGGGGGCGTCATCTGCTTCGCAATGGTA
TCCTACCAACCCCAATGATATGTTCGCTTCCTCAAATATTGTACCCCATCCTC
ATCCTTGCTTCCAGGGGGCACCATTATTGGAGACGGACTGGGGCCGAGTGA
TGGCTCATCCGGTTTATTCTCGTGGAATATGAGCGTTCCAAATGACTGGCAG
GTCAGGGATGTGACTGAAGAGCCTGGTAATTTCGTTTAGTGGACTCGAACCTC
AAGCAGTTTTCCCTGATCCTACTCTACCAAATGCCCTTGACAACACATTCGA
TGTGGTCCAACAACTACAAGACTCATCCTACCCTTCCTCTTCCTCTTTTGAAT
TCACACCCCCCGATTCATCAACGGCCGAGTCTAATCGGCGGGAAAAGAAAC
AAAATCCTCAGTGGAGCTTCTGCCTCGCTTCCGATAATACAGCTGATAAATA
TGCTCGTTCAACGATGACGCACAATTTGATCCGTATATACCACGACAGTATG
GAGAACGCGTTGTCATGCTGGTTGACGGAGCACAACTGCCCTTATACCGAT
AAAATAAGCAGCCTGCTGCCATTTAATGAAAGAAAGGAATGGGGTCCCAGC
TGGTCGAACAGGATGTGCATCCGGGTCTGTCGGTTGGACCGTGCATCCAGC
CAATACGTGGCCGGGCGTTGAGCGCGGAAGAGGACAAGACCCGCAGCCCGG
GCACTCCACCTGGCCATCATGGCATTTGCCTCACAGTGGACTCAGCATGCGC
AAAGAGGATCAGATTTATACGTCCCCGCGCCGATCGACTATGACGAGCGAT
CCATCCGTAAAAACGTTTGGAATGACGCGCGCCACGCCTTAGAGCACTCAA
CAAGGATACCCTCTTTCCGCATTATATTCGCAAACATCATATTCTCGTTAAC
CCAGAGTCCCTTGGACCATAGTCAAGACGAACGGCTGGGTCAGCTATTGGA
AACTGACAGTGCGCCTTTCTTTCTTGAAACCGCCAATCGCCAGCTTTACAAC
TTTAGACACAAGTTCGCCAGACTCCAACGGGAGGCACCTCCCTCTCCAAGT
GTGAGGGAGCTTCGGAGGGGGTCGGTAGGGTCGACAATGACTGATGTACTG
GAGATGCGACGTCTTCTGCTTCTGAGTCTCCCCAGGTTGATCCGATTCTCG
ATAGCCAGGACCACCGCACTACTCTCGGTCTTATGTTCTGGCTGGGGGTCAT
GTTTGACACCTTGAGTTCTGCAATGTACCAGCGACCATTAGTGGTATCAGAT
GAGGACAGCCAGATTGCATCAGCCTCGCCTCCGATAGCCGAACCGGAAGAG
CAAATCGACTTAGACTGCTTTAATATCCCCCAAAGTGGAGTGCGTAAAAAG
CAGGACGTATGGGGCGACTTTTTCCTCCGCAGTTCCCTTGAACGCCAGGAAT
CCACACAAATACAGATAAGATGGCCATGCTCCTACGAAGATGCTGCGGCCG
TTCTCTCCGAGGCAACACCCGTCAAAGTCCTGCTTTACCGCCGCATCACACA
ACTCCAAACCCTAATATACCGAGGGGCGAGTCCTGACCGACTTGAGGAAGC
CATTCAGAAGACTCTCCTAGTTTATCAGCACTGGAACTCCATATACCAGGGC
TTCATGCTCGACTGTGTCGCTAACCACGAATTCCTCCCTCCTCGTATTCAATC
GTGGTACGTGATTCTTGACGGCCACTGGCATGTCGCCACCATGCTTCTAGCA
GACATGTAGAAAGCATCGACAACGGACGGCTCGGTTCGAAGCTCGGCCGC
GAGGCTCGACAAGCCACAGACTTTGTCTCAAATCTACGAATTGATAATGCAT
TGGCGGTCGGTGCCCTTGCTCGTTCATCACTACACGGACAAGACCCCGTCAT
GCTCCGCTATTTCCACGATTCCCTTAACGAGGTGGCTTTCCTCGTTGAGCCG
TGGACAGTTGTTCTCGTCCATTGTTTCGCCAAGGCGGCATCTATCTCGCTGG
AAAGCATACATGTTATACCTGGCGAGCCCATGGACGTATTGTCGGAOAGAT
TCCGGCAGAACTGCGAGTCTGTATCTGTGCGCTTCAGTATCTTGCAAGGAA
GTCGGATATGGCTTTCTTGGTGTCAAGGAATTTGTCCAGGTCGTTGGATCTG
AAGCTTAGCCGGATGCCATGAAAAGGGCGAATTC
SEQ ID NO 47. Sequence of the promoter region of the alcR orthologue in A.
versicolor. The translation start site is at position 644:
GAATTTCCCAACGTCAATCAAGAGTTTGTTTTTAAGTGCTACGGAATATATCA
AAGCTCTCTCGTAAAGCACAGGTAATCCCTTCCCATGCGACTTCATCTTCAA
GTTTCAGCAATTGGAACACGATATGTCCATAATTAAGGAGGCCTGTGGATG
TGGAAGGGTTGGAGGAGGCCACCAATCCGGGGATGTCGAGCAACGATCAGC
ATTCGCCAAATCAACGTACCTCTCGTTAATTAGCTCTGATTAGTGTGATGAG
CTCTATATCACTCCGCCACCCGCTCGCTCTCGTCCTTCGTCCCCGGCAACTG
CTCCAYAGACTTGGAAGACRCCTCTCGGCTCGGCACCGTTCTCGCCCATCGG
TTCAATCCGCCGACTTTGATGCTTCAAATCTCCCAAAGATCCTTGGAAAATC
TATCTTCGCCCTCCAGATTGGGCAGCGGAACGTATCGCCGCCATACCGGTAC
CCCGACCCCACACTAGGCTTCCCCACCCGGACCCCGCACAGTTCGTGAYCTC
CTTGGGAGGAGCTGAAGCTGGGTGCCCCTGCGACAAGTTATCTGCGTCGGG
ATCCCGCTGTGTCTTCATCTCCTCGGAACCCAATGCAGAAGTCGTTATCA
AACTCGGTTGCTCGCCATGGATG
SEQ ID NO 48. flav walk3: CGAAGGACGTGGTGTGTCGATGCCAAC
SEQ ID NO 49. flav walk4: TTAGCACACAATFATGCGCACTCCATG
SEQ ID NO 50. Alcflav walk5: TTCCGCGTAGCCTTTGCCAATGTATTG
SEQ ID NO 51. Alcflav walk6: GAATGGAGCTCGACGAGCTCTTAGATC
SEQ ID NO 52. Alcflav walk7: CTGGACTGTCTCTCTCACCATACAGAG
SEQ ID NO 53. Alcflav walk8: GCGATGTTGCTCGCAGATACCGTTGAG
SEQ ID NO 54. Alcflav walkup1: TTGTGACTCAAGAGCCAGTCGAACGTG
SEQ ID NO 55. Alcflav walkup2: AGCAGTGTTGGAATTCCGTTCAAGCTC
SEQ ID NO 56. Sense PCR oligonucleotide (Alcflav for) for the amplification of the
ORF of the A. flavus alcR orthologue: ATGTCTTATCGTCGCCGTCAG
SEQ ID NO 57. Anti-sense PCR oligonucleotide (Alcflav rev) for the amplification of
the ORF of the A. flavus alcR orthologue TCAAAGGGCGCACATATGATAG
SEQ ID NO 58. Aspergillus flavus genomic DNA fragment encoding the full open
reading frame of the A. flavus alcR orthologue:
GAATTCGCCCTTATGTCTTATCGTCGCCGTCAGCATCGTAGTTGTGATCAAT
GTCGTAAAGGCAAGAGAGCATGCGATGCCCTCCTGGCTGACGAGCTTGAAC
GGAATTCCAACACTGCTGCTCGACAAGCGTACAATCACGCGTGCTCCAATTG
CAGAAAATACAAAAGAAAATGCACGTTCGACTGGCTCTTGAGTCACAAGGA
ATCCCGGCATGCTCATAGCAAGAGAGCCAGAAATATCGCGATCGCCCTCTC
GCGGCAGGTGAACGATTGTTCCGCTCATTCCTCTCAACAAACCTCCACTGGG
CGCAATCCTACAGAGCTCCCTCTGCAAAACATCGAGGATTGCGAATGGCCA
ACGTCTGTTAGGGACCCGCTTTTGCCGTTCCCACAAGACGAGGAACTAGATG
CGGACTGGTTAACTTGGGGATGCCTCAACGACGCAGTGTCCATCTCTCCTCT
AAGCGCCGACATGACTCTCAATGGGGATAGGCACGTCAATCCTAACCAGAC
ACCACAAATGAGTACTCAATGGAACTCTGTCGGGGCCGGCCAGGCATGGCA
AAGTATCGGTCAAACTTCACTGCTCGACACGATGAACAGTTCTAACTTCG
TCGCAATTCAAGGATACACCCGACTATCGATCAmGAGACATGGGATATCA
GTTCTGAGCTCCCGCTTCACGGTCTTCCACCTACCGAAGGACGTGGTGTGTC
GATGCCAACAAACAGTACACTGTGTGTGGGCTCAAACCAATTAGCACACAA
TTATGCGCACTCCATGATGACGCGCAACCTAATTCACATTTATAACGACAGT
ATGGAAAATGCATTGAGCTGTTGGCTGACCGAGCGTAATTGTCCCTACAGTG
CCCGGGGGTACGTTGACAAAACAGGGCCGAAGACAGGTCCTTATACCACGA
ATAGGATCTACAGACGAATTTGCCTCTTGGATAGGGCATGCTCATCCATCCC
GGGTCGACGTCTCACGAGTGTGGAGAGTAGAACAGCAACACAGACACTTCA
TGCTGTCATATGGCATFCGCTTCTCAGTGGCTGGAGAGGCCTTCAGCAGAC
AAAGATATCCCAATACCATCTTCTTCAGCTCACCACGAAAGTGGCATGCGTG
AGGGTCTCTGGAATGAAGCGCGTCATGCGCTGGAGAATTCGAGAGCAATTC
CATCGTTCCGCGTAGCCTTTGCCAATGTATTGTTTTCGCTGGCGCAACGACC
CCTACACGTTGAAGAAGGAATGGAGCTCGACGAGCTCTTAGATCACGATCC
TGCCCCAATGTATCTCGAAACGGGGCTTAGGCAGCTGTHACTTTTCGTTCT
AGATTGATTAAGCTTCGGCGGCAAGGTCCCAACCGAGCGCTCGAGCAATGC
TGCAAGGAGAGCAAAGGGGATAAAAGCACCCATCAGTTGAGCCAAATCGA
TCTGATGCTGAAGGACTCTGAAACCCATCACAGTTTCGATCTTCTATTCTGG
CTGGGCATCATGTTTGATACGTTGACAGCTGTCATATATCAACGTCCCCCGG
TCATTTCCGATGAGGACAGTCAGATCATACGCCCCCGGTCACGCTTCTCGTT
TCCGGACGCCGTTGATCGGATGGATGGGATATTAGCTCCTATTCCGCTAGC
CGACGTGAAGAAAGTGTATGGGGCGATCTTTTTCTTCGCAAACGTAACATGC
TCCACAATCTCAACCAGGCCCGCTGGCCTTGTTCTTACGAGGAGGCAGCAG
AAGTCTTGTCCGACGCCGCGCCAGTCAAGGTTCTCCTATTCCGTCGCATAAA
TCATATCAATACCCTGGTATGCCGGGGAGGTGGGGCAGAGGCCATTGAAGA
AGCGATCCACAGCGCTCTCTTGGTCTACGAGTATTGGAACTCGTCGTACAAG
CAGTTTATGCTGGACTGTCTCTCTCACCATACAGAGCTCCCGTCTCGCATAC
AATCATGGTATCTAGTGCTGGCTGGACATTGGCATGTTGCGGCGATGTTGCT
CGCAGATACCGTTGAGGAGATAGATCAAGCCCGACTTGGTCAAAACTCCCA
AACCGAACATCGGTATACCACAGGCCTCATCTCCGTCTTGCGTCACGAAAAT
GCTTTCGCTGTTGGAGGGCTCGCCCAATACTCTTACGACCTGCAGGGCTCGT
CGCACCCTAAACTCCGCAATTTHCACGATTCAGTCAATCAAGCGGCATCCTT
GACTGAGCCATGGACTGCTGTCCTTATCCACTCTTTTAGAAAAGCAGGTACT
ATCCTAATCAGAGAGATTGGCAGATTACAATGTGGTTACCAAATGCAGCAG
GAATCTTTCATGCTGGCGTATCAGCGTTGTGAACACTGTATAAAGGCACTCC
AGTGCCTGGGAAGAAAGTCAGATATGGCTCTGGCCGCAGCTCAGAGTCTAT
CAGACAGTCTCAACATGACACTGTTGCGACCCAGTCCTATTGATTCCTATCA
TATGTGCGCCCTTTGAAAGGGCGAATTC
SEQ ID.NO 59. Amino acid sequence encoded by ORF predicted from genomic DNA
sequence derived from A. flavus:
MSYRRRQERSCDQCRKGKRACDALLADELERNSNTAARQAYNHACSNCRKY
KRKGTFDWLLSHKESRHAHSKRARMAJALSRQVNDCSAHSSQQTSTGRNPTEL
PLQNIEDCEWPTSVRDPLLPFPQDEELDADWLTWGCLNDAVSISPLASADMTLNG
DRHVNPNQTPQMSTQWNSVGAGQAWQSIGQTSLLDTMNSSITSSQFKDTPDYR
SFETWDISSELPLHGLPPTEGRGVSMPNTTLCVGSNQLAHNYAHSMMTRNLIHI
YNDSMBNALSCWLTERNCPYSARGYVDKTGPKTGPYTTNPJYRRICLLDRACSS
IPGRRLTSVESRTATQTUIAVIAFASQWLERPSADKDWPSSSAHHESGMREG
LWNEARHALENSRAIPSFRVAFANVLFSLAQRPLHVEEGMELDELLDHDPAPM
YLETGLRQLFTFRSRLIKLRRQGPNRALEQCCKESKGDKSTHQLSQIDLMLKDSE
THHTFDLLFWLGIMFDTLTAVIYQRPPVISDEDSQIIRPRSRFSFPDAVDLDGWDI
SSYSADRREESVWGDLFLRKRNMLHNLNQARWPCSYEEAAEVLSDAAVKVLL
FRRINHINTLVCRGGGAEAIEEAIHSAILVYEYWNSSYKQFMLDCLSUHTELPSR
IQSWYLVLAGHWHLAAMLADTVEEIDQARLGQNSQTEHRYTTTGLISVLRHEN
AFAVGGLAQYSYDLQGSSHPKLRNFHDSVNQAASLTEPWTAVLIHSFRKAGTIL
IREIGRLQCGYQMQQESFMLAYQRCEHCIKALQCLGRKSDMALAAAQSLSDSL
NMTLLRPSPIDSYHMCAL.
SEQ ED NO 60. The sequence of the A. flavus alcR promoter region. The translation
start site is at position 416:
ATTCGCCCTTACtATAGGGCACGCGTGGTCGACGGCCCgGGCTGGTATCATC
AAACGCTGAAGTGGGTGGACGTTTGGAGGCAATGCTTGGTGTTCCACTGTCC
CATGACGTCTAATAACCTTGGTAGTTGCAATCCATGACTGATCAGGTTTT
CTGGAGTCTTCATTGTAGCATCCCGGCCACAAAGAACAAGTCGTAGCCAGT
GGGATTTGACAGGCTGAAAGTGACCTCAAGCGTAGGCATATCACGAACTAT
TAATTTAAAAGTAACCCCGACCCGATCTATACCCCGCAAACCCCCGCATTTC
CCCAGCTTAGTCCGTACTTTATTATCTCTCGGATCATGTTCACCTGAACTAT
TCTCCCAGAAACGGCCTACCTTGCTGTCGACTATAACACATTGCYGCAAATT
ATG
SEQ ED NO 61. Asperigillus ustus cDNA fragment encoding the full open reading frame
of the A. ustus alcR orthologue:
GAATTCGCCCTTCTCGAATGAAGATGGGAGACTCCCGTCGCCGCCAGAATC
ATAGCTGGATCCGTGTCGCAAGGGGAAACGAGGGTGTGATGCGCCTGAAAA
TCGAAGTGGAGATGGAACACCTGCTCGAATTGCAAGCGGTOGAAGAAGAA
ATGCACATTCAATTTCGTCTCGTCCAGGCGCGCAGATTCCCGCGTCGTCGGT
GCCAATGCCCGGTCAAAAGCGAAGTCCACCTCTACCCCTGCTGTCTCTAGCG
CTGCATCGGTAGCCACTTCTGCGGCTGCCCCTCCCACTCCCGATATGGCGAC
ATCCCTGCCATGCTAAACACGOGTATGGACATGGGCACGAATGAGTACGAT
GCTCTCCTTCATGACGGTTTGCGGTCGTCACACCTTGACCCTACGAGGCTTG
GGGATATGTTTGCTTTTACCTCGCCGTCTAGTTTCACGGCGGAGGCTTTGCA
TGCGCAGAGTGCTGTTGGCACAGAAGCCATCGCGTGGGATTCAGGGATTCC
AACAGACTGGTCTATCCCTTCGATGCCTCGTCGGAAAAGTCGTTCACTCCGC
TTGAGAGTCAGGCGGTCTTTCTTGCACAGGAGGATTCGAACCAGTTTGACGT
TATTCAGGAGTTGGAAGATGGCTCATCCGACAACTTCACACCACCGGGGCG
GAAACGCGACGAGGATAAGCGACGGAAATTTCAATGGGAGTTATGCATCGC
TTCCGACAAAACAGCCAACCAGGTTGGCCGATCGACAATGACGCGCAATCT
AATGCGGATATATCACGATAGCATGGAGAATGCGCTCTCATGTTGGTTGACC
GAGCACAACTGTCCGTATGCCGACCCGATGAGCGCAATGCTACCFTTTAACC
AGAGGAAAGAATGGGGTCCCAGTTGGTCGAACAGGATGTGTATCCGGGTCT
GTCATTTAGATCGGGAATCATCCTCGATACGCGGAAGGGCACTGAGCGTAG
ACGAGGACCGGACGGCCGCGCGGGCGCTGCATCTCGCAATTGTCGCATTCG
CCTCACAGTGGACGCAGCATGCCCAAAGGGGGACAGGGCTTTCGGTTCCGA
CTGATATCGCGTACGATGAACGGTCGATTCGAAAGAATATATGGAACGAGG
CGCGGCATGCTCTACAACACTCCACAGGGATCCCGTCTTTCCGGGTAATATT
CGCCAACMTATTTTCTCATTGACACAGAGTCCGCTGGACGAGAATCGACCT
GCGAAGCTAGGTCAGCTGTTGGAGAATGATGGTGCTCCCGTATTCCTAGAG
AACGCCAATCGTCAGCTCTACACATTCCGACACAAGTTCGCGAGACTCCAA
CGAGAGGCTCCCCCGCCTGTGGCTGGGCTGCGACGAGGTTCAATATCATCC
ACTCTCACTGACGTGGTGGAAGTTCCGACTCCTGAATCTCCACAGGTCGATC
CAATTCTCGCGAATCAAGACCACCGAAGCACACTCAGCCTCCTCTTCTGGCT
TGGAATCATGTfCGACACCCTCAGTGCAGCCATGTACCAGCGCCCTCTTGTC
GTCTCAGACGAAGATAGCCAAATCGCGTCCGCGTCCCCGTCGGCCTCAACC
AACCCCCGAGTCAACCTCAACTATTGGGAAATCCCAGACAGCAATCTCCCA
GCGAAAAACGACGTCTGGGGTGAATTTTTCCTTCAACCTGCCGCTCGCCAGG
AACTGGCCTCCGCACATCCCCAAATCCAACCAAAACAACCCCGTTGGCCGT
GTTCCTACGAAGAAGCCGCATCAGTCCTGTCCGAGGCAACACCGGTAAAAG
TCCTTCTCTACCGCCGCGTCACCCAACTCCAAACCCTTATCTACCGTGGCGC
GTCTCCCGCACGGCTGAAGAAGTCATTCAAAGAACGCTTCTCGTCTACCAC
CATTGGACCTGCACATATCAATCATTTATGCTCGACTGTGTGGCAAACCACG
AGTCCCTTCCACACCGTATTCAGTCTTGGTATGTTATTCTTGATGGCCATTGG
CACCTCTCCGCAATGCTTCTCGCCGATGTGCTAGAGTCCATCGACAGAAGCC
ACCTCGGACTCGAGTCGGAGCGCGAGTCCCGGATTGCAAGCCATCTTATTG
CAACACTGCGAATCGACAATGCACTCGCAGTCGGTGCCTTGGCTAGGGCAT
CGCTACACGGGGAGAATAGCATGATGCATCGACATTTCCATGACTCGTTGA
ACGAGGTCGCGTTCCTGGTGAGCCGTGGACAGTCGTTTTGGTCCATTGTTT
CGCGAAGGCGGCGGCTATTTCGCTGGATTGTCTGGGTCAGGGACAGGGAGG
TGCTTTGGCAGAATGTTTTCGGCAGAATTGTGAATATTGTATTTGTGCGTTG
AAGTATTTGGGACGGAAATCGGACATGGCGTTTTGTGTTGCGGGCGGGTTG
GAGAAGGAGTTGCTTGAGAAAGCTGGGAGTATGCTGTCAGCGGATATCCTT
GTGTAAAAGGGCGAATTTCC3′
SEQ ID NO 62. Amino acid sequence predicted from alcR cDNA sequence derived
from A. ustus.
MKMGDSRRRQNUSCDPCRKGKRGCDAPENRSGDGYTCSNCKRWKXKCTFNF
VSSRRADSRVVGANARSKAKSTSTPAVSTAASVATSAAAFPPDSGDIPAMLNT
GMDMGTNBYDALUDGLRSSHDPTRLGDMFAFTSPSSFTAEALHAQSAVGTE
AIAWDSGIPTDWSIPSMPRSEKSFTPLESQAVFLAQEDSNQFDVIQELEDGSSDNF
TPPGRKRDEDKRRKFQWELCIASDKTANQVGRSTMTRNLMRIYHDSMENALSC
LTEHNCPYADPMSAMLPFNQRKEWGPSWSNRMCIRVCBLDRESSSIRGRALS
VDEDRTAARALHLAIVAFASQWTQHAQRGTGLSVPTDIAYDERSIRKNIWNEAR
HALQHSTG1PSFRVWANFSLTQSPLDENRPAXLGQLLENDGAPVFLENANRQL
YTFRHKFARLQRBAPPPVAGLRRGSISSTLTDVLEVPTPESPQVDPILANQDHRST
LSLLFWLGIMFDTLSAAMYQRPLVVSDEDSQIASASPSASTNPRVNLNYWBIPDS
NLPAKNDVWGEFFLQPAARQELASAHPQIQPKQPRWPCSYEEAASVLSEATPV
KVLLYRRVTQLQTLIYRGASPARLEEVIQRTLLVYHHWTCTYQSFMLDCVANH
ESLPHRIQSWYVILDGHWHLSAMLLADVLESIDRSHLGLESERESRIASDLIATLR
IDNALAVGALARASLHGENSMMHRHFHDLNEVAFLVEPWTVVLVHCFAKAA
AISLDCLGQGQGGALAECFRQNCEYCICALKYLGRKSDMAFCVAGGLEKELLE
KAGSMLSADILV
SEQ ID NO 63. Oligonucleotide (Alcvers seq4r) for the generation of first strand cDNA
from mRNA encoding A. versicolor AlcR: CAANTTGTGCGTCATCGTTG
SEQ ID NO 64. Antisense PCR oligonucleotide (Alcvers seq5r) designed for the
amplification of A. versicolor cDNA encoding part of the alcR gene:
GGAAGCGAACATATCATTG
SEQ ID NO 65. Aspergillus versicolor cDNA fragment encoding the full open reading
frame of the A. versicolor AlcR orthologue:
ATGGATGACCCCCGCCGCCGCCAGTTTCATAGTTGTGACCCCTGTCGCAAGG
GCAAGAGGCGGTGTGATGCTCCGAGCAACCGGGAAAATGGTAACTTTGATT
CTTGCACTAACTGCAAGCGATGGAAGAAAGAGTGCACATTTACCTGGCTCT
CCTCGAAGCCAGCGAAGCGTGCGGACCCCAAAGGACGAGCAAGACCGAAA
CCGGGCGTTTCGACTACTTCTAGCAAACCTAGTGGTGCCAGCAACCCTAGCA
CTACTAGTAACCCTAGTAGTGATAGCGGTGGGACACCTCCTGATCCAAGTCG
CGTTGTCCCTTCCATGGTGGGCTCCTATAATGCCCTCGTGGACGGGGGGGCG
TCACTGCTTCGCAATGGTATCCTACCAACCCCAATGATATGTTCGCTTCCTC
AAATATTGTACCCCCATCCTCATCCTTGCTTCCAGGGGGCACCATTATTGGA
GACGGAGTGGGGCCGAGTGATGGCTCATCCGGTTTATTCTCGTGGAATATGA
GCGTTCCAAATGACTGGCAGGTCAGGGATGTGACTGAAGAGCCTGGTAATT
CGTTTAGTGGACTCGAACCTCAAGCAGTTTTCCCTGATCCTACTCTACCAAA
TGCCCTTGACAACACATTCGATGTGGTCCAACAAGTACAAGACTCATCCTAC
CCTTCCTCTTCCTGTTTTGAATTCACACCCCCGATTCATCAACGGCCGAGTCT
AATCGGCGGGAAAAGAAACAAAATCCTCAGTGGAGCTTCTGCCTCGCTTCC
GATAATACAGCTGATAAATATGCTCGTTCAACGATGACGCACAATTTGATCC
GTATATACCACGACAGTATGGAGAACGCGTTGTCATGCTGGTTGACGGAGC
ACAACTGCCCTTATACCGATAAAATAAGCAGCGTGCTGCCATTTAATGAAA
GAAAGGAATGGGGTCCCAGCTGGTCGAACAGGATGTGCATCCGGGTCTGTC
GGTTGGACCGTGCATCCTCTTCAATACGTGGCCGGGCGTTGAGCGCGGAAG
AGGACAAGACCGCAGCCCGGGCACTCCACCTGGCCATCATGGCATTTGCCT
CACAGTGGACTCAGCATGCGCAAAGAGGATCAGATTTATACGTCCCCGCCC
CGATCGACTATGACGAGCGATCATCCGTAAAAACGTTTGGAATGACGCGCG
CCACGCCTTAGAGCACTCAACAAGGATACCCTCTTTCCGCATTTATATTCGCA
AACATCATATTCTCGTTAACCCAGAGTCCCTTGGACCATAGTCAAGACGAAC
GGCTGGGTCAGCTATTGGAAACTTGACAGTGCGCCTTTCTTTCTTGAAACCGC
CAATCGCCAGCTTTACAACTTTAGACACAAGTTCGCCAGACTCCAACGGGA
GGCACCTCCCTCTCCAAGTGTGAGGGAGCTTCGGAGGGGGTCGGTAGGGTC
GACAATGACTGATGTACTGGAGATGCCGACGTCTTCTGCTTCTGAGTCTCCC
CAGGTTGATCCGATTCTCGATAGCCAGACCACCGCACTACTCTCGGTCTTAT
GTTCTGGCTGGGGGTCATGTTTGACACCTTGAGTTCTGCAATGTACCAGCGA
CCATTAGTGGTATCAGATGAGGACAGCCAGATTGCATCAGCCTCGCCTCCG
ATAGCCGAACCGGAAGAGCAAATCGACTTAGACTGCTTTAATATCCCCCAA
AGTGGAGTGCGTAAAAAGCAGGACGTATGGGGCGACTTTTTCCTCCGCAGT
TCCCTTGAACGCCAGGAATCCACACAAATACAGATAAGATGGCCATGCTCC
TACGAAGATGCTGCGGCCGTTCTCTCCGAGGCAACACCCGTCAAAGTCCTG
CTTTACCGCCGCATCACACAACTCCAAACCCTAATATACCGAGGGGCGAGT
CCTGAGCGACTTGAGGAAGCGATTCAGAAGACTCTCCTAGTTTATCAGCACT
GGAACTCCATATACCAGGGCTTCATGCTCGAGTGTGTCGCTAACCACGAATT
CCTCCCTCCTCGTNTTCAATCGTGGTACGTGATTCTTGACGGCCACTGGCAT
CTCGCCACCATGCTTCTAGCAGACATTGTAGAAAGCATCGACAACGGACGG
CTCGGTTCGAAGGCCGGCCGCGAGGGTCGACAAGCCACAGACTTTGTCTCA
AATGTACGAATTGATAATGCATTGGCGGTCGGTGCCCTTGCTCGTTCATCAC
TACACGGACAAGACCCCGTCATGCTCCGCTATTTCCACGATTCCCTTAACGA
GGTGGCTTTCCTCGTTGAGCCGTGGACAGTTGTTCTCGTCCATTGTTTTCGCCA
AGGCGGCATCTATCTCGCTGGAAAGCATAGATGTTATACCTGGCGAGCCCAT
GGACGTATTGTCGGAGAGATTCCGGCAGAACTGCGAGTTCTGTATCTGTGCG
CTTCAGTATCTTGCAAGGAAGTCGGATATGGCTTTCTTGGTGTCAAGGAATT
TGTCCAGGTCGTTGGATCTGAAGCTTAGCCGGATGCCATGAAAAGGGCGAA
TTC3′
SEQ ID NO 66. Amino acid sequence predicted from alcR cDNA sequence derived
from A. versicolor:
MDDPRRRQFHSCDPCRKGKRRCDAPSNRENGNFDSCTNCKRWKKECTFTWLS
SKPAKRADPKGRARPKPGVSTTSSKPSAASNFSTTSNPSSDSGGTPPDPSRVVPS
MVGSYNALVDGGASSASQWYPTNPNDMFASSNIYPPSSSLLPGGTTIGDGLGPS
DGSSGLFSWNMSVPNDWQVRDVTEEPGNSFSGLEPQAVFPDPTLPNALDNTFD
VVQQLQDSSYPSSSSFEFTPPDSSTAESNRREKKQNPQWSFCLASDNTADKYAR
STMTHNLIRIYHDSMENALSCWLTEHNCPYTDKISSLLPFNERXEWGPSWSNRM
CIRVCRLDRASSSIRGRALSAEEDKTAARALHLAIMAFASQWTQHAQRGSDLYV
PAPIDYDERSRKNYWNDARHALEHSTRIPSFRIIIFANIIFSLTQSPLDHSQDERLG
QLLETDSAPFFLETANRQLYNFRHKFARLQREAPPSPSVRELRRGSVGSTMTDV
LEMPTSSASESPQVDPILDSQDHRTTLGLMFWLGVMFDTLSSAMYQRPLVVSDE
DSQIASASPPIAEPEEQIDLDCFNWQSGVRKKQDVWGDFFLRSSLERQESTQIQIR
WPCSYEDAAAVLSEATPVKXTLLYRRTTQLQTLIYRGASPDRLEEAIQKTLLVYQ
HWNSIYQGFMLDCVANHEFLPPRIQSWYVILDGHWHLATMLLADIVESIDNGRL
GSKLGREARQATDFVSNLRIDNALAVGALARSSLHGQDPVMLRYFHDSLNEVA
FLVEPWTVVLVHCFAKAASISLESIHVIPGEPMDVLSERFRQNCEFCICALQYLA
RKSDMAFLVSRNLSRSLDLKLSRMP
SEQ ID NO 67 Degenerate oligonucleotide n-alcr2:
ATGGMWGAYMCGCGCCGMCGC
SEQ ID NO 68 Degenerate oligonucleotide c-alcR:
AASAAACGCATATCCGACTTCCT
SEQ ID NO 69 Degenerate oligonucleotide AlcRATG:
ATGGCAGATAGGCGCCGAC
SEQ ID NO 70 Degenerate oligonucleotide alcRTGA:
CTACAAAAAGCTGTCAACTTTCCC
SEQ ID NO 71: Degenerate oligonucleotide alcMID:
TCCGACATAAGTTTGCACGAATG
SEQ ID NO 72 Degenerate oligonucleotide alcMIDR:
CATTCGTGCAAACCTTATGTCGGA
SEQ ID NO 73 Sequence generated by degenerate PCR using consensus
oligonucleotides of A. bicolor:
TTGTGATCCGTGTCGGAAGGGGAAGAGGGGGTGCGATGGTCACCACTAGAA
TTTCATCTATGTCTTGAAGAATCAAGCTAATAAGTGTAGGAAACCGGACTGA
AATTCTCTCAATTCCTGCTCGAACTGCAAAAAATGGAAAAAGGAGTGCGC
GTTCAACTGGCTGGCCACAAATCCCACTATCAAAGGCAAGGGAAACCAGGA
AAAGAACAGGAGAACTAAAGCTAAGCCTAGTACTGCCGCGACTGATACAAA
TACGGCTATTGCTACGCCTGATGATAGTGTCGACATCCCTTCTG2TGGCAGT
GATGTTGGTATCAGCGTGGGCGATGGCTCCTACGGTAGUGTATCGATGATG
GACTTCAQTCTGCGCAGTGGTTTCCTGTTAATCCCGGCAACGGTGATGTGCT
CGCGCTGCCTGGGACTGGATTGTTTGACCTTACETCGTCTTCATTGTTGTTTC
CAGAAGGGGGTATCGGGGAAACGATACGAGTGACCCATATGCACAGTCTAT
AATTTCGTGGAACATGGGCGGGCTTTCCTGACAATTGGCAACTTGGTGCTGT
ACCTGGAAAGTGTTTCGCCAGACTTGACCTACCTACAAACTCGCTCGATGAC
ACATCGACATAATCCAACCACTCGAAGAAGATTCAAGCCGAAATTCGAGG
TAATTCCCATCCGGCTTCTGCATCGCCTCCGACAACACGGCCAAAGCCTACG
CTCGCTCAACAATGACACGCAACCTTCTCCGCATATACCACGGCAGCATGG
ATAACGCACTATCATGCTGGCTAACAGAGCATAACTGCCCGTACATTGACTC
AATCGGGCGACCTTCTACTACTATACAGCCAAAGAAAGGAATGGGGCCCGA
ACTGGTCAAATCGCATGTGCATCTGAGTTTGCCAATTAGATCGCGCATCCTC
TTCATTCGCAGTAGGGCATTGAGCGCAGAAGAGGACATGACTATGGTATT
TGCCTCGCAGTGGACTCAGCATGCGCAACGGGGACCGGTCCTATCTGTCCCT
GCGGGAATTGATGAAAATGAGAGATCAATTAGGAAGAATGTCTGGGATGAG
ATACGCCATGCGCAAGAGCATTCAACGAGGATTCCCTCGTTCCGGGTGATTT
ATGCGATTTGCGAATATCATCTTCTCGTTGACGCAGAGCCCGCTAGACAAAG
GCGAGGCGAGGTTAAGGACTGGGTCAGCTACTAGAGAATTACAGTGCACCG
ATATTCCTCGAGAACACCAACAGACAGCGATACCCCTTCCGACATAAGTTC
ACCAGGCTCCAGCGACGTAATCGGAGCTCGCCACAAGTCGACCCCATCCTA
TCCAGTCAGGACCACCGCGGTACGCTGAACCTGCTCTTCTGGTECGGAATCA
TGTTCGACACGCTAAGTGCAGCAATGTATCAACGCCCTGTCGTTGTCTCAGA
CGAGGATAGTGAAATCGCATCAATCTCACCTCCCCCTCCCACCCCCTCTCCA
CTCAACCCCCCAGCCCAAAATAACGTCGAGTGCTGGAACTTCCCCTCAGACC
AACCACAGACCACAACGCTAACCATCCGCTGAAAACAAGACGTCTGGGGCT
ACAGCTTCCTCCACCCAACAGCCTCCCTCTCACACCAAGAACCCACCACCCA
GCTCAACCCTCACCTCAGCCAAAACACCGCCCCAAACGCTGGCCCTGTACA
TACGCCGAATCAGCCTCGATTCTCTCCTTCGCAACCCCCGTAAAAGTCGTCC
TCTACCGGCGCGTCACCCAAGTCCAAACCCTCATCTACCGCGGCGCAGCACC
CTCGCAACTCGAATCCGTCATCCAGAAGACACTCCTCGTCTACAACCATTGG
CAGCAATTCTACGCGCCCTTCATGACAGACTACGTAACCAACCACGCTATTC
TCCCGCCGAGAATTCACTCCTGGTGTGTCATGTTAGACGGCCATTGGCATCT
CGCTGCGATGCTATTAGCCGTTGTAGTTGAGGAGACTGATAACGCCGGGCTT
GGGTTAGACYCTGCGCGAGAGGCAAGAAACYYATCGGATTTCGTCGGGACA
TTAAGGAGGGAGAACGCCTTAGCCGTTGGCGCGCTGCGAGGGCACCATTG
CAGGGCCAGAATCCGGGTATGGAAGAACATTACCATAATAGTTTGAACGAG
GTTGCGTTTCCGGTGGAGCCGTGGGCGGCTGTTCTGGTATATTGTTTTGCGA
AGGGGGGGGGGGGTATATTCCGCTTGAGAGGGTGGGTTATTCGTCGTTT
ACTAGGGATGGGTCTGGGGATGGCGTTAAGGACGGGAAGGTATTTCGGCTT
AATTGTGAGCTTTGTATTTGTGTTTCAGAGTATCTTGGAAGGAAGTCGGATA
TGCGTTTGTT
SEQ ID NO 74 Sequence generated by degenerate PCR using consensus
oligonucleotides of A. corrugatus:
ATGGATGACACGCGCCGCCGCCAGAATCATAGCTGCGACCCCTGTCGCAAG
GGCAAGCGACGCTGTGATGCCCCGGAAAATAGGAACGAGGCCAATGAAAA
CGGCTGGGTTTCGTGCTCAAATTGCAAGCGTTGGAACAAGGATTGTACCTTC
AATTGGCTGTCATCCCAACGCTCCAAGCCAAAAGGGGCTGCGCCCAGGGCG
AGGACGAAGAAGGCCAGAACTGCTACAACCACCAGTGAACCATCAACTTCA
GCTGCAGCAATCCCTACACCGGAAAGTGACAATCACGATGCGCCTCCAGTC
ATCAACGCTCACGACCCGCTCCCGAGCTGGACGCAGGGOCTGCTCTCCCAC
CCCGGCGACCTTTTTGATTTFAGCCAGTCGTGTATTCCCGCAAATGCAGAAG
ATGCAGCCAACGTACAGTCAGACGCACCTTTTCTGTGGGATCTAGCCATACC
CGGTGATTTCAGCATAGGCCAACAGCTCGAGAAACCACTCAGTCCGCTCAG
TTTTCAAGCAGTTCTTCTCCCGCCCCATAGCCCGAACACGGACGACCTCATT
CGCGAGCTGGAAGAGCAGACTACGGATCCGGACTCGGTCACCGATACTAAT
AGTCTACAACAGGTCGCTCAAGATGGGTCGCGATGGTCTGATCGGCAGTCG
CAGCTACTACCTGAGAACAGTCTGTGCATGGCCTCAGACAGCACAGCACGG
CGATATGCCCGTACCTCAATGACGAAGAATCTGATGCGAATCTACCACGAT
AGTATGGAGAATGCACTGTCCTGCTGGCTGACAGAGCACAACTGTCCATAC
TCCGACCAGATCACCTACCTGCCGCCCAAGCAGAGGGCGGAATGGGGCCCG
AACTGGTCAAACAGGATGTGCATCCGGGTGTGCCGGTTAGACCGTGTATCC
ACCTCATTACGTGGGCGCGCCTTGAGCGCTGAAGAGGATAGAGCCGCGGCA
CGAGCCCTGCACCTGGCTATCGTAGCCTTTGCGTCGCAATGGACGCAGCATG
CGCAGAGGGGGGCTGGGCTATCTGTTCCTGCAGACATAGCGGGCGATGAGA
GGGCCATCCGGAGGAACGCCTGGAATGAAGCACGCCATGCCTTGCAGCACA
CGACTGGAATTCCGTCGTTCCGGGTTATATTTGCGAATATCATCTTTTGTCTC
ACGCAGAGTGTGCTGGATGATACTGAGCAGCAGAATGTGGGTGCACGTCTG
GACAGOCTACTCGAGAATGACGGTGCGCCCGTCTTTCTGGAAACCGCGAAC
CGTCAGCTTTATACATTTCCGACATAAGTTTGCACGAATGCAACGCCGCGGTA
AGGCTTTCAACAGGCTCCCGGTGGAATCTGTCGCATCGACATTCGCCGATAC
TTTCGAGACACCGACGCCGCCGTCTGAAAGCCCCCAGCTTGACCCGGTFGTG
GCCAGTGAGGAGCATCGCAGTACATTAAGCCTTATGTTCTGGCTGGGGATC
ATGTTTGATAGTCTCAGCGCTGCAATGTACCAGCGACCACTGGTGGTGTCAG
ATGAGGATAGCCAGATATCATCGGCATATCCATCAACGCGCGGATCTGAAA
CGCCAATCAACCTAGACTGCTGGGAACCACCGAGACAGGCCCCGAGCAATC
AAGAAAAAAGCGACGTATGGGGCGACCTCTTCGTCCCGCACCTCGGAGTCTC
TCCAAGGTCACGAATCCCACACACAAATCTCCCAGCCAGCGGCTCGATGGC
CCTGCACCTACGAACAGGCCGCCGCCGCTCTCTCCTCTGCAACGCCAGTCAA
AGTCCTCCTCTACCGCCGCGTCACGCAGCTCCAAACCCTCCTCTATCGCGGC
GCCAGCCCTGCCCGCCTTGAAGCGGCCATCCAGAGAACGCTCCACGTCTAT
AATCATTGGACAGCAAAGTACCAACCATTTATGCAGGACTGCGTTGCTAAC
CACGAGCTCCTTCCTTCACGCATGCAGTCTTGGTACGTCATTCTAGACGGTC
ACTGGCATCTAGCCGCGATGTTACTAGCGGACGTTTTGGAGAGCATCGACC
GCGATGCGTACTCTGATATCAACCACATCGACCTCGTCACGAAGCTAAGGCT
CGATAATGCAGTGGCAGTTAGTGCCCTTGCGCGCTCTTCACTCCGAGGCCAG
GAGCTAGATCCGGGCAAAGCATGTCCGATGTATCGCCATTTUCCATGATTCTC
TGACCGAGGTGGCATTCCTGGTAGAACCGTGGACCGTCGTTCTTATTCACTC
ATTCGCCAAGGCTGCGTATATCTTGCTGGACTGTTTAGATCTGGACGGCCAG
GGAAATGCACTAGCGGGGTACCTGCAACTGCGGCAAAATTGCAACTACTGC
GTTCGGGCGCTGCAGTTTCTGGGCAGAAAGTCGGATATGGCGGCGCTGGTT
GCGAAGGATTTAGAGAGAGGTTTGAATGGGAAAGTTGACAGCTTTTTGTAG
SEQ ID NO 75 Sequence generated by degenerate PCR using consensus
oligonucleotides of A. cleistominutus:
GCCCTTTGTGATCCCTGTCGCAAGGGCAAGCGACGATGTGATGCCCCGGTA
GGTTGCCGATATCGGATCCGCAGCGTCTGCACCGACAGTCGCTGAGATGTA
ACACAGGAAAATAGAAACGAGGCCAATGAGAACAGCTGGGTTTCTTGCTCA
AATTGCAAGCGTTGGAACAAGGATTGTACCTTCAATTGCCTCTCGTCCCAGC
GCTCCAAGCCAAAAGGAGCTGCGCCCCGAGCCAGGACGAAGAAAGCCAGG
GCCGCTACAACCACCAGTGAACCATCAACTTCAGCTGCAGCTTTCCCTACAC
CGGAAAGTGACAATCACGATGCGCCTCCAGTCATCAACGCTCATGACGCGC
TCCCGAGCTGGACTCAGGGGCTGCTCCCACCCCAGCGACCTTTTCGATTT
CAGCCAGTCCTCTATTCCCGCAAATGTAGAAGATGCAGCAGCCAACGTGCA
GTCAGACGCACCTTTTCCGTGGGATCTGGCCATCCCCGGTGATTTCAGCATG
GGCCAACAGCTTGAGAAACCACTCAGTCCGCTCAGTTTTCAAGCATTCTTC
TCCCGCCCCATAGCCCGAACACGGATGACCTCATTCGCGAGCTGGAAGAGC
AGACAACGGATCCGGACTCGGTTACCGATACTAATAGTCTGCAACAGGCCG
CTCAACATGGGTCGCTATGGTCGATCGGCACTCGCCAGTGCTACCAGAGAA
CAGTGTGTGCATGGCCTCAGACAGCACAGCACGGCGATATGCCCGTTCCTC
AATGACGAAGAATCTGATGCGAATCTACCACGATAGTATGGAGAATGCACT
GTCCTGCTGGCTGACAGAGCACAATTGCCCATACTCCGACCAGATCAGCTA
CCTGCCGCCCAAGCAGAGGGCGGAATGGGGCCCGAACTGGTCAAACAGGAT
GTGCATCCGGGTGTGCCGGTTAGACCGCGTATCCACCTCATTACGCGGGCGC
GCCTTGAGCGCCGAAGAGGACAGAGCCGCAGCCCGAGCCCTGCATCTGGCG
ATCGTAGCCTTTGCATCGCAATGGACGCAACATGCGCAGAGGGGGGGTGAG
CTATCTGTTCCTGCACACATAGCGGCCGATGAGAGGGCCATCCGGAGGAAC
GCTTGGAATGAAGCACGCCATGCCATGCAGCACACGACAGGGATTCCCTCG
TTCCGGGTTATATTTGCGAATATCATCTTTTCTCTCACACAGAGTGTGCTGGA
TGATACTGAGCAGCAGGGTGTGGGTGCCCGTCTGGACAGGCTACTCGAGAA
TGACGGTGCGCCCGTCTTTCTGGAAACCGCGAACCGTCAGCTTTATACATTC
CGGCATAAGTTTGCACGGATGCAACGCCGCGGTAAGOGTTTCAACAGGCTC
CCGGGGGGATCTGTCGCATCGACATTCGCGGATATTTTCGAGACACCGACA
CCGTCGTCTGAAAGCCCCCAGCTTGACCCGGTTGTGGCCAGTGAGGAGCAT
CGCAGTACATTAAGCCTTATGTTTTGGCTAGGGATCATGTTCGATACCCTAA
GCGCTGCAATGTACCAGCGACCACTCGTGGTGTCAGATGAGGATAGCCAGA
TATCATCGGCATCTCCATCAACGCGCGGCTCTGAAACGCCAATCAACCTAG
ACTGCTGGGAACCACCGAGACAGGTCCCGAGCAACCAGGACAAAAGCGAC
GTATGGGGCGACGTCTTCCTCCGCGCCTCCGACTCTCTCCAAGATCACGAAT
CCCACACACAAATCTCCCAGCCAGCGGCTCGATGGCCCTGCACCTACGAAC
AGGCCGCCGCCGCGCTCTCCTCTGCAACGCCCGTCAAAGTCCTCCTCTACCG
CCGCGTCACGCAGCTCCAAACCCTCCTCTACCGCGGCGCCAGCCTGCCCGC
CTTTGAAGCGGCCATACAGAGAACGCTCCACGTCTATAATCACTGGACAGCA
AAGTACCAACCATTTATGCAGGACTGCGTTACTAACCACGAGCTCCTCCCTT
CGCGCATCCAGTCCTGGTACGTCATTCTAGACGGTCACTGGCATCTAGCCGC
GATGTTGCTAGCGGACGTTTTGGAGAGCATCGACCGCGAYfCGTACTCTGAT
ATCAACCACATCGACCTCGTCACAAAGCTAAGGCTCGATAACGCACTGGCA
GTTAGTGCCCTTGCGCGCTCTTCACTCCOAGGCCAGGAGCTAGACCCGGGC
AAAGCATCTCCGATGTATCGCCATTTCCATGATTGTCTGACCGAGGTGGCAT
TCCTGGTAGAACCGTGGACCGTCGTTCTTATTCACTCGTTCGCCAAGGCTGC
GTATATCTTGCTGGACTGTTTAAATCTGGACAGTCAGGGAAATGCACTTGCG
GGGTACCTGCAGCTGCGGCAAAATTGCCACTGCTGCATfCGGGCCCTGCAGT
TTCTGGGCAGGAAGTCGGATATGCGTTTGTTAAGGGC
SEQ ID NO 76 Sequence generated by degenerate PCR using consensus
oligonucleotides of A. faveolatus:
TGTGACCCCTGTCGCAAGGGCAAGCGACGCTGTGATGCCCCGGAAAATAGA
AACGAGGCCAATGAAAACGGCTGGGTTTCGTGCTCAAATTGCAAGCGTTTGG
AACAAGGATTGTACCTTCAATTGGCTCTCATCCCAACGCTCCAAGCCAAAAG
GGGCTGCACCCAGGGCGAGGACGAAGAAATCCAGGACCGCTACAACCACC
AGTGAACCAGCAACTTCAGCTGCAGCAATCCCTACACCGGAAAGTGACAAT
CACGATGCGCCTCCAGTCATCAACGCTCACGACGCGCTCCCGAGCTGGACT
CAGGGGCTGCTCTCCCACCCCGGCGACCTTTTCGATTTTAGTCACTCTGCTA
TTCCCGCAAATGCAGAAGATGCAGCCAACGTGCAGTCAGACGCACCTTTTC
CGTGGGATCTAGCCGTCCCTGGTGATTTCAGCATGGTCCAACAGCTCGAGAA
ACCACTCAGTCCGGTCAGTTTTCAAGCAGTTCTTCTCCCGCCCCATAGCCCG
AACACGGATGACCTCATTCGCGAGCTGGAAGAGCAGACTACGGATCCGGAC
TCGGTTACCGATACTAATAGTCTACAACAAGTCGCTCAAGATGGATCGCTAT
GGTCTGATCGGCAGTCGCCGCTACTACCTGAGAACAGTCTGTGCATGGCGTC
AGACAGCACAGCACGGCGATATGCCCGTTCCTCAATGACGAAGAATCTGAT
GCGCATCTACCACGATAGTATGGAGAATGCACTGTCCTGCTGGCTGACAGA
GCACAATTGTCCATACTCCGACCAGATCAGCTACCTGCCGCCCAAGCAGAG
GGCGGAATGGGGCCCGAACTGGTCAAAGAGGATGTGCATCCGGGTGTGCCG
GTTAGATCGCGTATCTACCTCATTACGCGGGCGCGCCTTGAGCGCCGAAGA
GGACAGAGCCGCAGCCCGAGCCCTGCATCTGGCGATCGTAGCTTTTGCTTCG
CAATGGACGCAGCATGCGCAGAGGGGGGCTGGGCTATCTGTTCCTGCAGAC
ATAGCGGCCGATGAGAGGGCCATCCGGAGGAACGCCTGGAATGAAGCACG
CCATGCCTTGCAGCATACGACGGGGATTCCGTCGTTCCGGGTTATATTTGCG
AATATCATCTTTTCTCTCACACAGAGTGTGATGGATGATAATGAGCAGCAGG
GTGTGGGTGCACGTCTGGACAAGCTACTCGAAAATGACGGTGCGCCCGTGT
TCCTAGAGACCGCGAACCGTCAGCTTTATACATTCCGGCATAAGTTTACACG
GATGCAACGCCGCGGTAAGGCTTTCAACAGGCTCCCGGGGGGATCTGTCGC
ATCGACATTCGCCGATATTTTCGAAACACCGACGCTGTCGTCTGAAAGCCCC
CAGCTTGACCCGGTTGTGGCCAGTGAGGAGCATCGCAGTACATTAAGCCT
ATGTTCTGGCTAGGGATCATGTTCGATACACTAAGCGCTGCAATGTACCAGC
GACCACTCGTGGTGTCAGATGAGGATAGCCAGATATCATCGGCATCTCCATC
AACGCGCGGCTCTGAAACGCCAATCAACCTAGACTGCTGGGAACCACCGAG
ACAGGTTCCGAGCAATCATGAAAACAGCGACGTATGGGGCGACCTCTTCCT
CCGCACCTCGGGCTCTCTCCAAGAGCACGAATCCCACACACAAATCTCCCA
GCCAGCGGCTCGATGGCCATGCACGTACGAACAGGCCGCCGCCGCTCTCTC
CTCTGCAACGCCTGTCAAAGTCCTCCTCTACCGCCGCGTCACGCAGCTCCAA
ACCCTCGTCTATCGCGGCGCCAGCCCTGCCCGCCTTGAAGCGGGTATCCAGA
GAACGCTTCACGTCTATAATCACTGGACAGCGAAGTATCAACCATTTATGCA
GGACTGTGTTGCTAAGCACGAGCTCCTTCCTTCGCGCATCCAGTCCTGGTAC
GTCATTTTAGATGGTCACTGGCATCTAGCCGCGATGTTGCTAGCGGACGTTT
TGGAGAGCATCGACCGCGATTCGTACTCTGATACCAACCACATCGACCTCGT
CACAAAACTAAGGCTCGATAATGCACTGGCAGTTAGTGCCCTTGCGCGCTCT
TCACTCCGAGGCCAGGAGCTAGACCCGGGCAAAGCATCTCCAATGTATCGC
CATTTCCATGATTCTCTGACTGAGGTGGCATTCCTGGTAGAACCGTGGACCG
TCGTTCTTATTCACTCGTTTGCCAAGGCTGCGTATATCTTGTTGGACTGTTTG
GATCTGGACGGCCAGGGAAATGCACTAGCGGGTTACCTGCAGGTGCGGCAA
AATTGCAACTACTGCATTCGGGCGCTGCAGTTTCTGGGCAGGAAGTCGGAT
ATGCGTTTGTTAA
SEQ ID NO 77 Sequence generated by degenerate PCR using consensus
oligonucleotides of A. heterothallicus:
TGTGATCCGTGTCGGAAGGGGAAGAGAGGGTGTGATGCGCTTGTGAGTTGT
GTCGTGCCTGTCTAACTGCTTGACCTGCCAGGATCATGCCATACCAGATCCC
GAGCTCGTCGGAGTCCAAACCTTTGTAACCATGATCCAGGAGATTCGAAGT
GGAGATGGATATACGTGGTCGATTGCAAACGATGGAAGAAGAAGTGCACT
TTTAATTTCGTCTCGTCGAGGCGCGCAGACGCCCGTAGTGTCGCTGCCATT
CTCGGGCAAAAGCGAAGCCCACTTCGACCCCTGTCGTCGCTACCACTGCATC
GGTAGCTACTTCTGTAGTGGCCCCTCCAACGCCAGATAGTGGCAACATCCCT
GCTATGCTGAATATGGGCATCAATACAAGTGAGTATAATGCACTGCTTGAC
GAGGGGTTGCGATCGTCGCAGCTTGACCCGGCAAGATTCGGAGACATGTTT
GAATTCATGTCGCCGTCGAACTTTGCTGCGGAGGTGTTGCATGCGCAGAGCG
CTATTGGGGGAGTGAACGAGACGCTCGCGTGGACTATGGGGGTTCCAGGAA
GTTGGCCGATGGGCATGATGCCGCAATCAGAAACGTCTTTTGAGTTCACTTCA
ATCGCAGGAGCTATTCATTTCGAACGAGGACGCGAACCCGTACGATGTTAT
GCAACAGTTGGAAGACGATTTCGAGGATCCTGCGACATCGGTCAGCAAACG
CGACGAAGATGTGCGAAAGTTCCAGTGGGAGTTATGTATCGCGTCAGACAA
AACAGCCAACAAGGTCGGCCGTTCGACGATGAATGGAAATTTGATCCGAAT
ATACCAGGACAGCATGGAAAACGCGCTGTCATGTTGGCTAACCGAACACAA
CTGTCCGTATGCCGACCCGATGAGCGCCATGTTACCGTTCAATCAAAGAAA
AGAATGGGGTCCAAGTTGGTCCAATAGAATGTGCATTGGGGFTTGTCGGTTA
GATCGTGCATCCTCGTCAATACGTGGGAGAGCATTGAGCGTAGAGGAAGAT
AGGACTGCGGCACGGGCCCTTCATCTCGCAATTGTTGCCTTCGCCTCACAAT
GGACGCAACATGCGCAGAAAGGAACGGGTTTATCAGTTCCGGCAGGCATCG
CATATGACGAGCGGTCGACTCGCAAAAATATCTGGAACGAGGCGCGGCACG
CGTTGCAACATTCAACTGGTATTCCGTCATTCAGGGTGGTATTTGCCAACAT
CATTTTCTCCCTTACGCAGAGTCCGCTGGACGAGACTCGGCCTGCAAAGGTTG
GCGCAGCTATTAGACAACGACGGCGCGCCTGTGTTTCTAGAAAATGCGAAC
CGTCAGGTTTACACATTTCGGCATAAATTTGCAAGACTACAGCGCGAAGCTC
CTCCACGTGCCGCGACAGACCTCCGACGAGGTTCGATATCATCCACACTCAC
CGAGGTGCTGGAGATTCCGACTCCAGAAAGTCCGCAACTTGACCCCATCCT
CGCCAGCCAAGACCATCGCAGCACACTAAGTCTCCTATTTTGGCTTGGAATC
ATGTTCGACACGCTCAGTTCCGCAATGTACCAGCGCCCACTAGTTGTCTCCG
ACGAAGACAGCCAGATCGGCTGCGCGTCCCCAACAGCTTCAGCCGACCATC
GAGTCAACCTCAACTACTGGGAAATCCCAGACAACGACCTTCCGGCGAAGA
ACGATGTCTGGGGCGAATTCTTCCTCCAACCCGCAGCACGTCAAGAGCCAA
CCTCCACACATCCTCAACTCCAACCACAACAACCTCGCTGGCCGTGCTCTTA
TGAAGAAGCGGCCTCTGTCCTCTCCGAAGCGACACCGGTCAAAGTCGTCCTT
TACCGCCGCATCACTCAACTCCAAACCCTCATCTACCGTGGCTCTTCTCCAG
CTCGTCTTGAAGAAGTTATCCAAAAGACCCTGCTTGTGTACCACCACTGGAC
ATGCACGTATCAATCCTTTATGCTCGACTGTGTCGCAAACCACGAATCCCTG
CCGCATCGAATTCAATCATGGTATGTTATCCTCGACGGCCAFCGGCACGTGG
CTGCGATGCTTCTTGCCGATGTGCTCGAGTCAATTGACAGAAGCTACCTCGG
TATGGAATCGGAGCGGGAATCCCGAATCGCAAGCGACCTCATCGCAACACT
TCGCATCGACAACGCACTCGCGGTCGGAGCACTAGCCCGCGCATCGCTGCA
TGOCCAGAATAGCACGATGCATCGCTACTTTCATGACTCGTTGAACGAGGTC
GCGTTCCTCGTCGAACCATGGACGGTTGTGCTAATTCATTCATTTGCGAAGG
CGGCGTATATTTCTCTCGATTGTTTGGGCCAGGGACAGGGCGGAGCATTAGC
AGAGTGTTTCCGGCAGAATTGCGAATATTGTATTTGTGCGCTGAAGTATTTG
GGGAGGAAGTCGGATATGCGTTTGTT
SEQ ID NO 78 Sequence generated by degenerate PCR using consensus
oligonucleotides of A. navahoensis:
GCCCTTTGTGATCCGTGTCGGAAAGGGAAGCGACGCTGTGATGCACCGGAA
AATAGGAACGAGACCAATGAGAACGGCTGGGCTTCTTGCTCGAATTGCAAA
CGTTGGAATAAGGATTGTACTTTCAACTGGCTGTCGTCGCAGCGCTCCAAGC
CTAAGGGGGCTGCACCCCGGGCGAGGATGAAGAAAGCCAGGACCGCTGCA
GCCACGGCTGAGCCATCAAATTCGGCTACCGCAATGCCTACACCGGAAAGT
GGCCATCAAGATACACCTCCTATTATTAACGCCTACGATGCGCTACCGAGCT
GGAGTCAGGGATTGGTCTCCCACCCCGGCGACCTGTTTGATTTCAGCCAATC
TTCTATTCCCATGCACACAGATGATGCGGTGAACGTGCAGTCAGAGGTGCCC
TTCCCATGGGATGTGGCTATTCCGGGCGACTTCAGCAGCATGGGCCAGCAGC
TCGAAAACCCCCTCAGTCCGCTCAGTTTTCAAGCAGTTATTCTCCCGCGTCA
CAGTCCGAACACGGATGACCTGATCCACGAGCTGGAAGAACAGTCAACGGA
CTCTACTAAGTTTGCTGGCCTACGGCGGGATACTCCTGATGGGTCGCTGTGG
TCTAGTCGGGCCTCGCCGCTAGCACCCCAGAACAGCTTGTGCATTGCATCAG
ACAAAACAGCACAGCAATATGCTCGTTCGTCGATGACAAAGAATCTGATGC
GCATCTATCATGACAGCATGGAGAATGCACTGTCTTGCTGGCTGACGGAGC
ACAACTGCCCCTACTCCGACCAGACCAGCTACCTGCCGCCCAAACAGAGGG
CGGAATGGGGTCCGAACTGGTCGAACAGGATGTGCATCCGGGTGTGCCGGC
TAGACCGCGTATCCACCTCATTACGCGGGCGGGCCGTGAGCGCAGAAGAGG
ACAGAGCCGCAGTCCGAGCCCTGAATCTGGCCATCGTAGCCTTTGCCTCGCA
ATGACGCAGCATGCGCAGAAGGGAGCTGGGCTATCTATTCCTACAGACAT
AGCAGGCGATGAGCGGGCCATCCGGAGAAACACCTGGAACGAGGCACGTC
ATGCCTTGCAGCGCTCGACTGGGATCCCCTCGTTCCGGGTCATATTTGCGAA
CATCATCTTTTCTCTGACACAGAGTGTGCTGGACGATAGTGAACAGCAGGGT
GCGGGTACACGTCTAGACAAGTTACTCGAGAATGACCGTGCGCCTTTGTTCC
TGGAAACCGCCAATCGTCAGCTCTGCACATTCCGGCATAAGTTTGCACGGAT
GCAACGTCGAAGGTCGACTGCCGACCAGCTCCGAAGGGTATCAGCAGCATC
CGCGCTTGCGGATATTTTCGAGACACCGACGCCGTCGCCTGGAAGCCCCCAT
CTCGACCCGATTCTAGCCAACGAGGAGCACCGCAGTACACTAAGCCTTATG
TTCTGGCTGGGGATCATGTTCGACACACTGAGTGCTGCAATGTACCAGCGAC
CACTTGTGGTGTCAGATGAGGATAGTCAGATATCATCGGCATCCCCGTCAAC
ACAGGGTTCTGAAACCCCAATCAAGCTAGACTGCTGGGAGCCACCAAGACA
GATTGCAAACGATCGAGCTAAAAGTGACGTATGGGGCGACCTCTTCCTGCG
CGACTCCGACTCCCCCCAGCACGACAAATCTCGCGCCCAGATCTCTCAGCCA
GCGGCTCGATGGCCCTGCACCTATGAACAAGCCGCCGCCGTTCTCTCCTCCG
CAACCCCCGTCAAAGTCCTCCTCTACCGCCGTGTCACACAGCTCCAAACCCT
CCTCTATCGCGGCGCCAGTCCGGCCCGCCTGGAAGCAGCCATACAGAAAAC
GATCCATGTGTACCAACACTGGACAGAAAAATACCAGCCCTTCATGCAGGA
GTGCGTCGCTAACCACGAGCTCCTTCCGTCGCGCATCCAGTCGTGGTACGTC
ATCCTTGACGGCCACTGGCACTTAGCTGCGATGCTGCTAGCCGATGTTCTGG
AGAGCATTGACCGCGACACGTACTCCGATATCGACCACACCGATCTCGTTA
CAAAACTAAGACCGATAATGCGCTGGCAGTTTAGCGCCCTTGCGCGCTCTTC
ACTCAGAGACCAGGAGCAATGTCCAGCACAAAGCATCTCAGATGTATCGCCA
TTTCCACGACTCTTTGACCGAAGTTGCCTTCCTGGTAGAGCCGTGGACTGTC
GTAGTTATCCACTCGTTTGCCAAGGCTGCGTATATCCTCCTGGACTGTTTGG
ATGTAGACGGGCAGCGAAGTACCCTGGCTGGGTATCTGCAGCTGCAGCAGA
ATT
GCAATTACTGCATTCGGGCGCTGCAGTATTTGGGCAGGAAGTCGGATATGC
GTTTGTTAAGGGC
SEQ ID NO 79 Sequence generated by degenerate PCR using consensus
oligonucleotides of A. spectabilis:
GCCCTTTGTGATCCGTGTCGGAAAGGGAAGAGGGGGTGCGATGCGCCTGAA
AACCGAACTGAAATCCTCTTCAGTTCCTGCTCGAACTGCAAAAAGTGGAAA
AAGGAGTGCACGTTCAACTGGCTGTCCACAAATCCCACCATCAAGGCCAAG
GGAAACCAGGAAAAGAAAAGGAGAAAAACTAAAGCGAAGCCTTGTACTGT
CGCGGCTGATACAAGTACGGATACTGCTACTCCTGATGATAGTGTCGGCATC
CCTTCAATTGGCAGTGATGTTGGCATCAGCGTGGGCGATGGCTCTTATGGTG
GCTTTATCGATGATGGAGTTCAGTCTGCGCAGTGGTTCCCTGTCAATCCGGG
AGACGGTGATGTGTTCGCGTTGCCCGGGACTGGGTTGTTGGACTTGCCTTCG
TCTTCGTTGTTGTTTTCAGAAGCAGGTATCGGGGGAAACGATACGAGTGACC
CATATGCACAGTCTTTAGTCTCGTGGAACATAGGCTTTCCTGACAGTTCGCA
ACTTGACGCTGTACCTGGAAAGTCTTTCACCAGACTTGACTCTGTACCTACA
GACTCTCTCGATTACAGATTCGACGTGATCCAACAACTCGAAGAAGAATTA
GCCCAAGATTCGAGGACATTCCCATCCGGCTTCTGCATGGCCTCCGACAACA
CGGCCAAAGCCTACGCTCGCTCAACAATGACCCACAACCTTCTCCGCATATA
CAACGACGGCATGGAGAACGCACTATCATGCTGGCTAACAGAGCATAACTG
CCCGTACACCGACTCAATCGGCGACCTTCTGCTACCATACAGCCAAAGAAA
GGAATGGGGCCCGGACTGGTCGAATCGCATGTGTATCCGAGTTTGCCACTTA
GATCGCGCATCCTCTTTGATTCGCGGTAGGGCGTTGAGCGCAGAAGAGGAC
AAGACTGCAGCTCGAGCGCTGCATCTAGCGATTGTGGCATTTGCCTCGCAGT
GGACTCAGCATGCGCAACGGGGACCGGTCCTATCTGTCCCTGCGGGAATTG
ATGAAGATGAGAGGTTAATTAAGAAGGATGTCTGGAATGAGGCACGCCATG
CGCTGGAGCACTCTACGAGGATTCCCTCGTTCCGGGTGATCTHGCGAATAT
CATCTTCTCGTTGACGCAGAGTCCGCTAGACAAAGGCGACAGGCGAGATCA
AGGACTGGGTCAGCTACTAGAGAACGACAGCGCACCAATATTCCTCGAGAA
CGCCAACAGACAGCTATACACCTTCCGGCACAAGTTCACCAAGCTCCAGCG
AAGTAATCGGAACTCGCCACAAGTCGATCCCATCCTATCTAGTCAGGACCA
CCGCAGTACGCTGAACCTGCTCTTCTGGCTCGGAATCATGTTCGACACGCTA
AGTGCAGCAATGTACCAACGCCCTCTCGTTGTCTCAGACGAGGATAGTCAG
ATCACATCAATCTCACTCCTCCCACACCGGCTCCACTCAACTCCCCAGCCC
AAATCAACCTCGACTGCTGGGACCTCCCCTCAGACCAACCACAGACCACAA
CGCTAACGTTGCGCCAAAAGCAAGACGTTTGGGGCGACTTTCTTCCTCCACCC
ATCACCCTCCCTCTCACACCAAGAACCCACCACCCAGCTCAACCCTCACCCT
CAGCTAGAACACCCCAAACGGTGGCCCTGCACATACGCCGAACCAGCCTCG
ATCCTCTCCTCTGCAACCCCCGTAAAAGTCCTCCTCTACCGGCGCGTCACCC
AACTCCAAAACCTCATCTACCGCGGTGCAACACCCTCGCAACTCGAATTTAGT
CATCCAGAAGACACCCTCGTCTACAACCACTGGCAGCAAACCTTACGCGCC
CTTCATGACAGACTGCGTGACCAACCACGCTATTCTCCCGCCGAGAATCCAA
TCCTGGTATGTCATTTTAGACGGCCATTGGCATCTCGCTGCGATGTTATTGG
CCGAAGTAGTTGAGGAAATCGATAACGCTAGGCTAGGGTTAGACTCTGCGC
GAGAGACAAGAAACATATCGAATTTTCGTCGAGACGTTAAGAAGGGAGAATG
CATTAGCCGTTGGCGCGCTAGCCAGCGCGTCACTGCAGGGTCAGAATCCCG
GTATGGAAGAACGTTACCATGATAGTGTGAATGAGGTTGCGTTTCTGGTGGA
GCCGTGGACGGTTGTTCTGGTGAATTGTTTTGCGAAGGGCGGGTATATTTCG
GCTGAGAGGGCTGCGGGTTGTTCGTCGTTTACTGGGGCTGGGGTTGGAGCTG
GGGATGGGATTGGCGTTGGAGAGGTGTTTCGTCTGAATTGTGGATTCTGTAT
TTGTGCGTTGGAGTATCTTGGTAGGAAGTCGGATATGCGTTTGTTAAGGGC
SEQ ID NO 80 Sally Three: GTCGACGAATTCGCCCTTCTCGAATG
SEQ ID NO 81 Sally Four: GTCGACGAATTCGCCCTTTTACACAA
SEQ ID NO 82 Sally14: CCATTTGCCCAGCTATCTGTC
SEQ ID NO 83 Alcust seq10r: TGCGCGTCATTGTCGATC
SEQ ID NO 84 NPT2-2: TCGCCTTCTATCGCCTTCTTG
SEQ ID NO 85 p35S-3: CTCGCCGTAAAGACTGGCGAACAG
SEQ ID NO 86 Sally 21: GTCGACGAATTCGCCCTTATGG
SEQ ID NO 87 Sally 22: GTCGACGAATTCGCCCTTAACTAC
SEQ ID NO 88 Alcfum scq4r: GACAAGCTCTGCTGGTAG
SEQ ID NO 89 Sally 12: GTCGACGAATTCGCCCTTTTCATGGC
SEQ ID NO 90 Sally 13: GTCGACGAATCGCCCTTGGTTGCTC
SEQ ID NO 91 M13 for: GTAAAACGACGGCCAG
SEQ ID NO 92 M13rev: CAGGAAACAGCTATGAC
SEQ ID NO 93 Alcvers seq2: CGCCTTAGAGCACTCAAC
SEQ ID NO 94 Alcvers seq1r: AGTGTGTGGCTTGTCGAG
SEQ ID NO 95 Alcvers seq5r: GGAAGCGAACATATCATTG
SEQ ID NO 96 Knpflav for: GGTACCGAATTCGCCCTTATGTCTTATC
SEQ ID NO 97 flavkpnI rev-2: GGTACCTCAAAGGGCGCACATATGATAG
SEQ ID NO 98 Alcflav seq8r: CCATTGAGAGTCATGTCG
SEQ ID NO 99 Sally 17P: CAGGGTACCCGGGGGTCGACCGGGCTGCAG
SEQ ID NO 100 Sally 18P:
CTGCAGCCCGGTCGACCCCCGGGTACCCTG
SEQ ID NO 101. DNA sequence of alcR gene from A. nidulans var. acristatus
ATGGCAGATACGSGCCGACGCCAGAATCATAGCTGYGAYCCCTGTCGCAAG
GGCAAGCGGCGCTGTGAYGCCCCGGTAGGTTGCCGATATCGGCTCCCCAGC
GTGTSCACTGATAGTCACTGAGACGTAACACAGGAAAATAGAAATGAGGCC
AATGAAAATGGCTGGGTTTCGTGCTCAANTTGCAAGCGTTGGAACAAGGAT
TGTACCTTCAATTGGCTCTCATCCCAACGCTCCAAGGCAAAAGGGGCTGCAC
CCAGGGCGAGAACGAAGAAAGCCAGGACTGCAACAACCACCAGTGAACCA
TCAACTTCAGCTGCAACAATCCCTACACCGGAAAGTGACAATCACGATGCG
CCTCCAGTCATCAACGGTCACGACGCGCTCCCGAGCTGGACTCAGGGGCTG
CTCTCCCACCCCGGCGACCTTTTCGATTTCAGCCAGTCTGCTATFCCTGCGAA
TGCAGAAGATGCAGCCAACGTGCAGTCAGACGCACCTTTTCCGTGGGATCT
AGCTATTCCCGGTGATTCAGCATGGGCCAACAGCTCGAGAAACCACTCAG
TCCGCTCAGTTTTCAAACAGTCCTTTTCCCGCCCCATAGCCCGAACACGGAT
GACCTCATTCGCGAGCTGGAAGAGCAGACTACGGATCCGGACTCGGTTACC
GATACTAAGAGTGTGCAACAGGTCGCTCAAGATGGTTCGATATGGTCTGAT
CGGCAGTCGCCGCTACTGCCTGAGAACAGTCTGTGCATGGCCTCAGACAGC
ACAGCACGGCGATATGCGCGTTCCTCAATGACGAAGAATCTGATGCGCATC
TACCACGATAGTATGGAGAATGCAGTATCCTGCTGGCTGACAGAGCACCAAT
TGTCCATACTCCGACCAAATCAGCTACCTGCCGCCCAAGCAGAGGGCGGAA
TGGGGCCCGAACTOGTCAAACACGGATGTGCATCCGGGTGTGCCGGTTAGAT
CGCGTATCTACCTCACTACGCGGGCGCGCCTTGAGTGCCGAAGAGGACAGA
GCCGCAGCCCGAGCCCTGCATCTGGCGATCGTAGCTTTTGCGTCGCAATGGA
CGCAGCATGCGCAGAGGGGGGCTGGGTTATCTGTTCCTGCAGACATAGCGG
CCGATGAGAGGGCCATCAGGAGGAACGCCTGGAATGAAGCACGCCATGCCT
TGCAGCACACGACGGGGATTCCGTCATTTCGGGTTATATTTGCGAATATCAT
CTTTTCTCTCACGCAGAGTGTGCTGGATGATAATGAGCAGCAGGGTGTGGGT
GCACGTCTGGACAAGCTACTCGAAAATGACGGTGCGCCCGTGTTCCTGGAA
ACTGCGAACCGTCMGCTTTATACATTCCGRCATAAGTTTGCACGAATGCAAC
GCCGCGGTAAGGCTTTCAACAGGCTCCCGGGGGGATCTGTCGCATCGACAT
TCGCCGGTATTTTCGAGACACCGACGCCGTCGTCTGAAAGCCCACAGYTTGA
CCCGGTTGTGGCCAGTGAGGAGCATCGCAGTACATTAAGCCTTATGTTCTGG
CTTGGGAWCATGTTCGATAGACTAAGCGCTGCAATGTACCAGCGACCACTC
GTGGTGTCAGACGAGGATAGCCAGATATCATCGGCATCTCCATCAACGCGC
GGCTCTGAAACGCCGATCAACCTAGACTGCTGGGAACCCCCAAGACAGGTC
CCGAGCAATCAAGAAAAGAGCGACGTATGGGGCGACCTCTTCCTCCGCACC
TCGGAGTCTCTCCCAGATCACGAATCCCACACACAAATCTCTCAGCCAGCGG
CTCGATGGCCCTGCACCTACGAACAGGCCGCCGCCGCTCTCTCCTCTGCAAC
GCCGGTCAAAGTCCTCCTCTACCGCCGCGTCACGCAGCTCCAAACCCTTCTC
TATCGCGGCGCCAGCCCTGCCCGCCTTGAAGCGGCCATCCAGAGAACGCTC
CATGTTTATAATCACTGGACAGCGAAGTACCAACCATTTATGCAGGACTGCG
TTGCTAACCACGAGCTCGTCCCTTCGCGCATCCAGTCTTGGTACGTCATTCT
AGACGGTCACTGGCATCTAGCCGCGATGTTGCTAGCGGACGTTTTGGAGAG
CATCGACCGCGATTCGTACTCTGATATCAACCACATCGACCTTGTCACAAAG
CTAAGGCTCGATAATGCATTAGCAGTTAGTGCCCTTGCGCGCTCTTCACTCC
GAGGCCAGGAGCTAGACCCGGGCAAAGCATCTCCGATGTATCGCCATTTCC
ATGATTCTCTGACCGAGGTGGCATTCCTGGTAGAACCGTGGACCGTCGTCCT
TATTCACTCGTTTGCCAAGGCTGCGTATATCTTGCTGGACTGTTTAGATCTGG
ACGGCCAAGGAAATGCACTAGCGGGGTACCTGCAGCTGCGACAAAATTGCA
ACTACTGCATTCGGGCGCTGCAGTTTCTGGGCAGGAAGTCGGATATGGCGG
CGCTGGTTGCGAAGGATTTAGAGACAGGTTTGAATGGGAAAGTTGACAGCT
TTTTGTAG
SEQ ID NO 102. DNA sequence of alcR gene from A. nidulans var. dentatus
ATGGCATGATACGCGCCGACGCCAGAATCATAGCTGCGAYCCGTGTCGCAA
GGGCAAGCGACGCTGTGATGCCCCGGTAGGTTGCCGATATCGGCTCCCCAG
CGTGTGCACTGACAGTCGCTGAGATGTAACACAGGAAAATAGAAACGAGGC
CAATGAAAACGGCTGGGTTTCGTGTTCAAATTGCAAGCGTTGQAACAAGGA
TTGTACGTTCAACTGGCTCTCATCCCAACGCTCCAAGGCAAAAGGGGCTGCA
CCTAGAGCGAGAACAAAGAAAGCCAGGACCGCAACAACCACCAGTGAACC
ATCAACTTCAGCTGCAACAATCCCTACACCGGAAAGTGACAATCACGATGC
GCCTCCAGTCATAAACTCTCACGACGCGCTCCCGAGCTGGACTCAGGGGCT
ACTCTCCCACCCCGGCGACCTTTTCGATTTCAGCCACTCTGCTATTCCCGCA
AATGCAGAAGATGCGGCCAACGTGCAGTCAGRCGCACCTTTTCCGTGGGAT
CTAGCCATCCCCGGTGATTTCAGCATGGGCCAACAGCTCGAGAAACCTGTC
AGTCCGCTCAGTTTTCAAGCAGTCGTCCTTCCGCCCCATAGCCCGAACACGG
ATGACCTCATTCGCGAGCTGGAAGAGCAGACTACGGATCCCGGACTCGGGTA
CCGATACTAATAGTGTACAACAGGTCGCTTCAAAACGGATCGCTATGGTCTG
ATCGGCAGTCCCCGCTACTGCCTGAGAACAGTCTGTGCATGGCCTCAGACA
AGCACAGCACGGCGATATGCCCGTTCCCCAATGACGAAGAATCTGATGCGA
ATCTACCCCGATAGTATGGAGAATGCACTGTCCTGCTGGCTGACAGAGCAC
AATTGTCCATACTCCGACCAGATCAGCTACCTGCCGCCCAAGCAGCGGGCG
GAATGGGGCCCGAACTGGTCAAACAGGATGTGCATCCGGGTGTGCCGGCTA
GATCGCGTATCTACCTCATTACGCGGGCGCGCCCTGAGTGCGGAAGAGGAC
AAAGCCGCAGCCCGAGCCCTGCATCTGGCGATCGTAGCTTTTGCGTCGCAAT
GGACGCAGCATGCGCAGAGGGGGGGTGGGCTAAATGTCCTGCAGACATAG
CCGCCGATGAGAGGTCCATCCGGAGOAACGCCTGGAATGAAGCACGCCATG
CCTTGCAGCACACGACAGGGATTCCATCTTCCGGGTTATATTTGCGAATAT
CATCTTTTCTCTCACGCAGAGTGTGCTGGATGATGATGAGCAGCACGGTATG
GGTGCACGTCTAGACAAGGTACTCGAAAATGACGGTGCGCCCGTGTTCCTG
GAAACCGCGAACCGTCAGCTTTATACATTCCGACATAAGTTTGCACGAATGC
AACGCCGCGGTAAGGCTTTCAACAGGCTCTCGGGAGGATCTGTCGCATCGA
CATTCGCCGGTATTTTCGAGACACCGACGCCGTCGTCTGAAAGCCCACACGCT
TGACCCGGTTGTGGCCAGTGAGGAGCATCGCAGTACATTAAGCCTTATGTTC
TGGCTAGGGATCATGTTCGATACACTAAGCGCTGCAATGTACCAGCGACCA
CTCGTGGTGTCAGATGAGGATAGCCAGATATCATCGGCATCTCCACCAAGG
CGCGGCGCTGAAACGCCGATCAACCTAGACTGCTGGGAGCCCCCGAGACAG
GTCCCGAGCAATCAAGAAAAGAGCGACGTATGGGGCGACCTYTTCCTCCGC
ACCTYGGACTCTCGCAGATCACGAATCCCACACACAAATCTCTCAGCCAG
CGGCTCGATGGCCCTGCACCTACGAACAGGCCGCCGCCGCTCTCTCCTNTGC
AACGCCCGTTAAAGTCCTCCTCTACCGCCGCGTSACGCAGCTYCAAACCCCT
CCTCTATCGCGGCGCCAGCCCTGCCCGCCTTGAAGCGGCCATCCCAGAGAA
CGCTCTACGTTTTATAATCACTGGACAGCGAAGTACCAACCATTTATGCAGG
ACTGYGTTGCTAACCACGAGCTCCTCCCTTCGCGCATCCAGTCTTGGTACGT
CATTCTAGACGGTCACTGGCATCTAGCCGCGATGTTGCTAGCGGACGTTTTG
GAGAGCATCGACCGCGATCGTACTCTGATATCAACCACATCGACCTTGTAA
CAAAGCTAAGGCTCGATAATGCACTAGCAGTTAGTGCCCTTGCRCGCTCTTC
ACTCCGAGGCCAGGAGCTGGACCCGGGCAAAGCATCTCCGATGTATCGCCA
TTTCCATGATTCTCTGACCGAGGTGGCATTCCTAGTAGAACCGTGGACCGTC
GTTCTTATTCACTCGTTTGCCAAAGCTGCGTATATCTYGCTGGACTGTTTAGA
TCTGGACGGCCAAGGAAATGCACTAGCGGGGTACCTGCAGCTGCGGCAAAR
TTGCAACTACTGCATTCGAGCGCTGCAATTTCTGGGCAGGAAGTCGGATATG
SSKKYGYTGGTTGCGAAGGATTTAGAGAGAGGTTTGAATGGGAAAGTTGAC
AGCTTTTTGTAG
SEQ ID NO 103. DNA sequence of alcR gene from A. nidulans var. vuimellin
CCCTTTGTGATCCGTGTCGGAAGGGGAAGCCACGCTGTGATGCCCCGGTAG
GTTGCCGATATCGGGTCCCCAGCGTGTGCACTGACAGTCGCTGAGATGTAAC
ACAGGAAAATAGAAACGAGGCCAATGAAAACGGCTGGGTTTCGTGTTCAAA
TTGCAAGCGTTGGAACAAGGATTGTACCTTCAATTGGCTCTCATCCCAACGC
TCCAAGGCAAAAGGGGCTGCACCTAGAGCGAGAACAAAGAAAGCCAGGAC
CGCAACAACCACCAGTGAACCATCAACTTCAGCTGCAACAATCCCTACACC
GGAAAGTGACAATCACGATGCGCCTCCAGTCATAAACTCTCACGACGCGCT
CCCGAGCTGGACTCAGGGGCTACTCTCCCACCCCGGCGACCTTTTCGATTTC
AGCCACTCTGCTATTCCCGCAAATGCAGAAGATGCGGCCAACGTGCAGTCA
GACGCACCTTTTCCGTGGQATCTAGCCATCCCCGGTGATTTCAGCATGGGCC
AACAGCTCGAGAAACCTCTCAGTCCGCTCAGTTTTCAAGCAGTCCTTCTTCC
GCCCCATAGCCCGAACACGGATGACCTCATTCGCGAGCTGGAAGAGCAGAC
TACGGATCCGGACTCGGTTACCGATACTAATAGTGTACAACAGGTCGCTCA
AGATGGATCGCTATGGTCTGATCGGCAGTCGCCGCTACTGCCTGAGAACAG
TCTGTGCATGGCCTCAGACAGCACAGCACGGCGATATGCCCGTTCCACAAT
GACGAAGAATCTGATGCGAATCTACCACGATAGTATGGAGAATGCACTGTC
CTGCTGGCTGACAGAGCACAATTGTCCATACTCCGACCAGATCAGCTACCTG
CCGCCCAAGCAGCGGGCGGAATGGGGCCCGAACTGGTCAAACAGGATGTGC
ATCCGGGTGTGCCGGGTAGATCGCGTATCTACCTCATTACGCGGGCGCGCCC
TGAGTGCGGAAGAGGACAAAGCCGCAGCCCGAGCCCTGCATCTGGCGATCG
TAGCTTTTGCGTCGCAATGGACGCAGCATGCGCAGAGGGGGGCTGGGCTAA
ATGTTCCTGCAGACATAGCCGCCGATGAGAGGTCCATCCGGAGGAACGCCT
GGAATGAAGCACGCCATGCCTTGCAGCACACGACAGGGATTCCATCATTCC
GGGTTATATTTGCGAATATCATCTTTTCTCTCACGCAGAGTGTGCTGGATGA
TGATGAGCAGCACGGTATGGGTGCACGTCTAGACAAGCTAGTCGAAAATGA
CGGTGCGCCCGTGTTCCTGGAAACCGCGAACCGTCAGCTTTATACATTCCGA
CATAAGTTTGCACGAATGCAACGCCGCGGTAAGGCTTTCAACAGGCTCCCG
GGAGGATCTGTCGCATCGACATTCGCCGGTATTTTCGAGACACCGACGCCGT
CGTCTGAAAGCCCACAGCTTGACCCGGTTGTGGCCAGTGAGGAGCATCGCA
GTACATTAAGCCTTATGTTCTGGCTAGGGATCATGFTCGATACACTAAGCGC
TGCAATGTACCAGCGACCACTCGTGGTGTCAGATGAGGATAGCCAGATATC
ATCGGCATCTCCACCAAGGCGCGGCGCTGAAACGCCGATCAACCTAGACTG
CTGGGAGCCCCCGAGACAGGTCCCGAGCAATCAAGAAAAGAGCGACGTAT
GGGGCGACCTCTTCCTCCGCACCTCGGACTCTCTCCCAGATCACGAATCCCA
CACACAAATCTCTCAGCCAGCGGCTCGATGGCCCTGCACCTACGAACAGGC
CGCCGCCGCTCTCTCCTCTGCAACGCCCGTCAAAGTCCTCCTCTACCGCCGC
GTCACGCAGCTCCAAACCCTCCTCTATCGCGGCGCCAGCCCTGCCCGCCTTG
AAGCGGCCATCCAGAGAACGCTGTACGTTTATAATCACTGGACAGCGAAGT
ACCAACCATTTATGCAGGACTGCGTTGCTAACCACGAGCTCCTCCCTTCGCG
CATCCAGTCTGGTACGTCATTTCTAGACGGTCACTGGCATCTAGCCGCGATG
TTGCTAGCGGACGTTTTGGAGAGCATCGACCGCGATTCGTACTCTGATATCA
ACCACATCGACCTTGTAACAAAGCTAAGGCTCGATAATGCACTAGCAGTTA
GTGCCCTTGCGCGCTCTTCACTCCGAGGCCAGGAGCTGGACCCGGGCAAAG
CATCTCCGATGTATCGCCATTTCCATGATTCTCTGACCGAGGTGGCATTCCT
GGTAGAACCGTGGACCGTCGTTCTTATTCACTCGTTTGCCAAAGCTGCGTAT
ATCTTGCTGGACTGTTTAGATCTGGACGGCCAAGGAAATGCACTAGCGGGG
TACCTGCAGCTGCGGCAAAATTGCAACTACTGCATTCGGGCGCTGCAATTTC
TGGGCAGGAAGTCGGATATGCGTTTGTTAAGGGC
SEQ ID NO 104 Consensus Amino acid motif 1 CDPCRKGKXCD
SEQ ID NO 105 Consensus Amino acid motif 2 CXNCKXWXKXCXF
SEQ ID NO 106 Consensus Amino acid motif 3 NALSCWLTEHINCPY
SEQ ID NO 107 Consensus Amino acid motif 4 WSNMRCI(X)₀₋₁RVCXLDR
SEQ ID NO 108 Consensus Amino acid motif 5 RXRALS(X)₂ED
SEQ ID NO 109 Consensus Amino acid motif 6 FASQWTQHAQ
SEQ ID NO 110 Consensus Amino acid motif 7 RHA(X)₄TXIPSFR
SEQ ID NO 111 Consensus Amino acid motif 8 FANLIIFSLTQS
SEQ ID NO 112 Consensus Amino acid motif 9 FLE(X)₂NR(X)₄FRHKF
SEQ ID NO 113 Consensus Amino acid motif 10 MFDTLS
SEQ ID NO 114 Consensus Amino acid motif 11 AMYQRPLVVSDEDSQI
SEQ ID NO 115 Conscnsus Amino acid motif 12 DVWG(X)₂FL
SEQ ID NO 116 Consensus Amino acid motif 13 ATPVKVLLYRR
SEQ ID NO 117 Consensus Amino acid motif 14 LDGHWHL
SEQ ID NO 118 Consensus Amino acid motif 15 NALAVXALAR
SEQ ID NO 119 Consensus Amino acid motif 16 EVAFXVEPW(X)₂VL
SEQ ID NO 120 Consensus Amino acid motif 17 LXRKSDM
SEQ ID NO 121 A. nidulans alcR nucleic acid sequence
ATGGCAGATACGCGCCGACGCCAGAATCATAGCTGCGATCCCTGTCGCAAG
GGCAAGCGACGCTGTGATGCCCCGGAAAATAGAAACGAGGCCAATGAAAA
CGGCTGGGTTTCGTGTTCAAATTGCAAGCGTTGGAACAAGGATTGTACCTTC
AATTGGCTGTCATCCCAACGCTCCAAGGCAAAAGGGGCTGCACGTAGAGCG
AGAACAAAGAAAGCCAGGACCGCAACAACCACCAGTGAACCATCAACTTC
AGCTGCAACAATCCCTACACCGGAAAGTGACAATCACGATGCGCGTCCAGT
CATAAACTCTCACGACGCGCTCCCGAGGTGGACTCACGGGCTACTCTCCCAC
CCCGGCGACCTTTTCGATTTCAGCCACTCTGCTATTCCCGCAAATGCAGAAG
ATGCGGCCAACGTGCAGTCAGACGCACCTTTTCCGTGGGATCTAGCCATCCC
CGGTGATTTCAGCATGGGCCAACAGCTCGAGAAACCTCTCAGTCCGCTCAGT
TTTCAAGCAGTCCTTCTTCCGCCCCATAGCCCGAACACGGATGACCTCATTC
GCGAGCTGGAAGAGCAGACTACGGATCCGGACTCGGTTACCGATACTAATA
GTGTACAACAGGTCGCTCAAGATGGATCGCTATGGTCTGATCGGCAGTCGC
CGCTACTGCGTGAGAACAGTCTGTGCATGGCGTCAGACAGCACAGCACGGC
GATATGCCCGTTCCACAATGACGAAGAATCTGATGCGAATCTACCACGATA
GTATGGAGAATGCACTGTCCTGCTGGCTGACAGAGCACAATTGTCCATACTC
CGACCAGATCAGCTACCTGCCGCCCAAGCAGCGGGCGGAATGGGGCCCGAA
CTGGTCAAACAGGATGTGCATCCGGGTGTGCCGGCTAGATCGCGTATCTACC
TCATTACGCGGGCGCGCCCTGAGTGCGGAAGAGGACAAAGCCGCAGCCCGA
GCCCTGCATCTGGCGATCGTAGCTTTTTGCGTCGCAATGGACGCAGCATGCGC
AGAGGGGGGCTGGGCTAAATGTTCCTGCAGACATAGCCGCCGATGAGAGGT
CCATCCGGAGGAACGCCTGGAATGAAGCACGCCATGCCTTGCAGCACACGA
CAGGGATTCCATCATTCCGGGTTATATTTGCGAATATCATCTTTTTCTCTCACG
CAGAGTGTGCTGGATGATGATGAGCAGCACGGTATGGGTGCACGTCTAGAC
AAGCTACTCGAAAATGACGGTGCGCCCGTGTTCCTGGAAACCGCGAACCGT
CAGCTTTATACATTCCGACATAAGTTTGCACGAATGCAACGCCGCGGTAAG
GCTTFCAACAGGCTCCCGGGAGGATCTGTCGCATCGACATTCGCCGGTATTT
TCGAGACACCGACGCCGTCGTCTGAAACGCCCACAGCTTGACCCGGTTGTGG
CCAGTGAGGAGCATCGCAGTACATTAAGCCTTATGTTCTTGGCTAGGGATCAT
GTTCGATACACTAAGCGCTGCAATGTACCAGCGACCACTCGTGGTGTCAGAT
GAGGATAGCCAGATATCATCGGCATCTCCACCAAGGCGCGGCGCTGAAACG
CCGATCAACCTAGACTGCTGGGAGCCCCCGAGACAGGTCCCGAGCAATCAA
GAAAAGAGCGACGTATGGGGCGACCTCTTCCTCCGCACCTCGGACTCTCTCC
CAGATCACGAATCCCACACACAAATCTCTCAGCCAGCGGCTCGATGGCCCT
GCACCTACGAACAGGCCGCCGCCGCTCTGTCCTCTGCAACGCCCGTCAAAGT
CCTCCTCTACCGCCGCGTCACGCAGCTCCAAACCGTCCTCTATCGCGGCGCC
AGCCCIGCCCGCCTTGAAGCGGCCATCCAGAGAACGCTCTACGTTTATAATC
ACTGGACAGCGAAGTACCAACCATTTATGCAGGACTGCGTTGCTAACCACG
AGCCCTCCCTTCGCGCATCCAGTCTTGGTACGTCATTCTAGACGGTCACTG
GCATCTAGCCGCGATGTTGCTAGCGGACGTTTTGGAGAGCATCGACCGCGA
TTCGTACTCTGATATCAACCAACATCGACCTTGTAACAAAGCTAAGGCTCGAT
AATGCACTAGCAGTTAGTGCCCTTGCGCGCTCTTCACTCCGAGGCCAGGAGC
TGGACCCGGGCAAAGCATCTCCGATGTATCGCCATTTCCATGATTCTCTGAC
CGAGGTGGCATTCCTGGTAGAACCGTGGACCGTCGTTCTTATTCACTCGTTT
GCCAAAGCTGCGTATATCTTGCTGGACTGTTTAGATCTGGACGGCCAAGGA
AATGCACTAGCGGGGTACCTGCAGCTGCGGCAAAATTGCAACTACTGCATT
CGGGCGCTGCAATTTCTGGGCAGGAAGTCGCATATGGCGGCGCTGGYTGCG
AAGGATTTAGAGAGAGGTTTGAATGGGAAAGTTGACAGCTTTTTG
SEQ ID NO 122 A. nidulans AlcR polypeptide sequence
MADTRRRQNTTSCDPCRKGKRRCDAPENRNEANBNGWVSCSNCKRWNKDCTF
NWLSSQRSKAXGAAPRARTKKARTATTTSEPSTSAATTFTPESDNHDAPPVINSH
DALPSWTQGLLSHFGDLFDFSHSAIPANAEDAANVQSDAPFPWDLAIPGDFSMG
QQLEKPLSPLSFQAVLLPPHSPNTDDLIRELEEQTTDPDSVTDTNSVQQVAQDGS
LWSDRQSPLLPENSLCMASDSTARRYARSTMTKNLMRIYHDSMENALSCWLTE
HNCPYSDQISYLPPKQRAEWGPNWSNRMCIRVCRLDRVSTSLRGRALSAEEDK
AAARALHLAIVAFASQWTQHAQRGAGLNVPADIAADERSIRRNAWNEARHAL
QHTTTGIPSFRVIFANIIFSLTQSVLDDDEQHGMGARLDKLLENDGAPVFLETANR
QLYTFRHKFARMQRRGKAFNRLPGGSVASWAGIFETPTPSSESPQLDPVVASEE
HRSTLSIMFWLGIMFDTLSAAMYQRPLVVSDEDSQISSASPPRRGAETPINLDC
WEPPRQVPSNQEKSDVWGDLFLRTSDSLPDHESETQISQPAARWPCTYEQAAA
ALSSATPVKVILYRRVTQLQTLLYRGASPARLEAAIQRTLYVYNHWTAKYQPF
MQDCVANHELLPSRIQSWYVILDGHWHLAAMLLADVLESIDRDSYSDINHIDLV
TKLRLDNALAVSALARSSLRGQELDPGKASPMYRHFHDSLTEVAFLVEPWTVV
LIHSFAKAAYILILCLDLDGQGNALAGYLQLRQNCNYCIRALQFLGRKSDMAAL
VAKDLERGLNGKVDSFL
SEQ ID NO 123 Consensus amino acid sequence of the AlcR orthologues.
CXASDXTA(X)₄R(X)₂M(X)₂NLXRIY(X)₃MXNALSCWLTEHNCPYXD(X)₄₋₆
L(X)₄RXEWGPXWSNRMCI(X)₀₋₁VCXLDRXS(X)₀₋₁S(X)₁₋₂RXRALS(X)₂ED(X)₄₋₁₃
ASQWTQHAQXG(X)₂L(X)₂P(X)₂I(X)₃ER(X)₆W(X)₃RHA(X)₄TXIPSFR(X)₂₋₄
FANIIFSLTQS(X)₂D(X)₉₋₁₃LL(X)₄APXFLE(X)₂NR(X)₄FRHKF(X)₃QR(X)₄₋₃₃
SP(X)₂DP(X)₆HRXTLXLXFWXGXMFDTLSXAMYQRPLVVSDEDSQI
SEQ ID NO 124 Amino acid sequence of A. nidulans var. dendatus AlcR protein:
MADTRRRQNHSCDPCRXGKRRGDAPENRNEANENGWVSCSNCKRWNXDCTF
NWLSSQRSKAKGAAPRARTKKARTAYTTSEPSTSAATTPTPESDNIWAPPWNSH
DALPSWTQGLLSHPGDLFDFSHSAIPANABDAANYQSXAPFPWDLAWGDFSMG
QQLEKPLSPLSFQAVLLPPHSPNTDDLIRELEEQTTDPDSGTDTNSVQQVAQNGS
LWSDRQSPLLPENSLCMASDSTARRYARSPMTKNLMRIYPDSMBNALSCWLTE
HNCPYSDQISYLPPKQRAEWGPNWSNRMCIRVCRLDRVSTSLRGRALSAEEDK
AAARALAIVAFASQWTQHAQRGAGLNVPADLAADERSLRRNAWNEARHAL
QHTTGIPSFRVIFANIIFSLTQSVLDDDEQHGMGARLDKLLENDGAPVFLETANR
QLYTFRHKFARMQRRGKAFNRLSGGSVASTFAGIFETPTPSSESPQLDPVVASEE
HRSThSLMFWLGIMFDTLSAAMYQRPLVVSDEDSQISSASPPRRGAETPINLDC
WEPPRQVPSNQEKSDVWGDLFLRTXDSLPDHESHTQISQPAARWPCTYEQAAA
ALSXATPVKVLLYRRVTQLQTLLYRGASPARLEAAIPENALLPPKQRAEWGPNT
FYNHWTAKYQPFMQDCVANUELLPSRIQSWYVILDGIIWIPAAMIIADVLESI
DRDSYSDINHIDLVTKLRIDNALAVSALARSSLRGQELDPGKASPMYRHFHDSL
TEVAFLVEPWTVVUHSFAKAAYIXLDCLDLDGQGNALAGYLQLRQXCNYCIR
ALQFLGRKSDXXLVAKDLERGLNGKVDSFL
SEQ ID NO 125 Amino acid sequence of A. nidulans var. acristatus AlcR protein
MADTXRRQNHSCDPCRKGKRRCDAPENRNEANENGWVSCSNCKRWNKDCTP
NWLSSQRSKAKGAAPRARTKKARTATTTSEPSTSAATIPTPESDNHDAPPVINA
HDALPSWTQGILSHIPGDLFDFSHSAIFANAEDAANYQSDAPFPWDLAIPGDFSM
GQQLEKPLSPLSFQTVLFPPHSPNTDDLIRELEEQTTDPDSVTDTKSVQQVAQDG
SIWSDRQSPLLPENSLCMASDSTARRYARSSMTKNLMRIYHDSMENALSCWLT
EHNCPYSDQISYLPPKQRAEWGPNWSNRMCIRVCRLDRVSTSLRGRALSAEED
RAAARALHLAIVAFASQWTQHAQRGAGLSVPADIAADERAIRRNAWNBARHA
LQHTTGIPSFRVIFANIIFSLTQSVLDDNEQQGVGARLDKLLENDGAPVFLETANR
XLYTFRHKFARMQRRGKAFNRLPGGSVASTFAGIFETPTPSSESPQXDPVVASEE
HRSTLSLMFWLGXMFDTLSAAMYQRPLVVSDEDSQISSASPSTRGSETPINLDC
WEPPRQVPSNQEKSDVWGDLFLRTSDSLPDHESHTQISQPAARWPCTYEQAAA
ALSSATPVXVLLYRRVTQLQTLLYRGASPARLEAAIQRTLHVYNHWTAKYQPF
MQDCVANHELLPSRIQSWYVILDGHHLAAIMLLADVLESIDRDSYSDINHIDLV
TKLRLDNALAVSALARSSLRGQELDPGKASPMYRHFHDSLTEVAFLVEPWTVV
LIHSFAKAAYILLDCLDLDGQGNALAGYLQLRQNCNYCIRAILQFLGRKSDMAAL
VAKDLETGLNGKVDSFL
SEQ ID NO 126 Amino acid sequence of A. nidulans var. vuimellin AlcR protein:
CDPCRKGKRRCDAPENRNEANENGWVSCSNCKRWNKIDCTFNWLSSQRSKAK
GAAPRARTKKARTATTTSEPSTSAATTPTPESDNHDAPPVLNSIIDALPSWTQGLL
SHPGDLFDFSHSAIPANABDAANVQSDAPFPWDLAIPGDFSMGQQLEKPISPLSF
QAVLLPPHSPNTDDLIRELEEQTTDPDSVTDTNSVQQVAQDGSLWSDRQSPLLP
ENSLCMASDSTARRYARSTMTKNLMRIYHDSMENALSCWLTEHNCPYSDQISY
LPPKQRAEWGPNWSNRMCIRVCRIDRVSTSLRGRALSAEEDKAAARALHLAIV
AFASQWTQHAQRGAGLNVPADIAADERSIRRNAWNEARHALQHTTGIPSFRVIF
ANIIFSLTQSVLDDDEQHGMGARLDKLLENDGAPVFLETANRQLYTFRHKFAR
MQRRGKAFNRLPGGSVASTFAGIFETPTPSSESPQLDPVVASEEHRSThSLMFWL
GIMFDTLSAAMYQRPLVVSDEDSQISSASPPRRGAETPTILDCWEPPRQVPSNQE
KSDVWGDLFLRTSDSLPDHESHTQISQPAARWPCTYEQAAAALSSATPVKVLLY
RRVTQLQTLLYRGASPARLEAAIQRTLYVYNHWTAKYQPFMQDCVANHELLPS
RIQSWYVILDGHLAAMLLADVESIDRDSYSDINHIDLVTKLRLDNALAVSM
LARSSLRGQELDPGKASPMYRHFEDSLTEVAFLVEPWTVVLIHSFAKAAYILLD
CLDLDGQGNALAGYLQLRQNCNYCIRALQFLGRKSDMRLLR
SEQ ID NO 127 Amino acid sequence of A. faveolatus AlcR protein:
CDPCRKGKRRCDAPENRNEANENGWVSCSNCKRWNXDCTTNWLSSQRSKPK
GAAPRARTKKSRTATTTSEPATSAAAIPTPESDNHDAPPVINAHDALPSWTQGL
LSHPGDLFDFSHSAIPANAEDAANVQSDAPFPWDLAVPGDFSMVQQLRKPLSPL
SFQAVLPPHSPNTDDLIRELEEQTTDPDSVTDTNSLQQVAQDGSLWSDRQSPLL
PENSLGMASDSTARRYARSSMTKNLMRIYHDSMENALSCWLTEHNCPYSDQIS
YLPPKQRABWGPNWSNRMCIRVCRLDRVSTSLRGRALSAEEDRAAARALHLAI
VAFASQWTQHAQRGAGLSVPADIAADERAIRRNAWNBARHALQHTTGIPSFRV
IFANIIFSLTQSYMDDNEQQGVGARLDKLLENDGAPVFLETANRQLYTFRHKFT
RMQRRGKAFNRLPGGSVASTFADIFETPTTSSESPQLDPVVASEEHRSTLSLMFW
LGIMFDTLSAAMYQRFLVVSDEDSQISSASPSTRGSETPINLDCWEPPRQVPSNH
ENSDVWGDLFLRTSGSLQEHIESITTQISQPAARWPCTYEQAAAALSSATVKVLL
YRRVTQLQTLLYRGASPARLEAAIQRTLHVYNHWTAKYQPFMQDCVANHELL
PSRIQSWYVILDGHWHLAAMLLADVLESIDRDSYSDTNHIDLVTKLRLDNALAV
SALARSSLRGQELDPGKASPMYRHFHDSLTEVAFLVBPWTVVUHSFAKAAYIL
LDCLDLDGQGNALAGYLQLRQNCNYCIRALQFLGRKSDMRLL
SEQ ID NO 128 Amino acid sequence of A. corrugatus AlcR protein:
MDDTRRRQNHSCDPCRKGKRRCDAPENRNEANENGWVSCSNCKRWNXDCTF
NWLSSQRSKPKGAAPRARTKKARTMTTSEPSTSAAAIPTPESDNHDAPPVINA
HDPLPSWTQGLLSHIGDLFDFSQSSIPANAEDAANYQSDAPFLWDLAIPGDFSIG
QQLEKPLSPLSFQAVLLPPHSPNTDDLIRELEEQTTDPDSVTDTNSLQQVAQDGS
RWSDRQSQLLPENSLCMASDSTARRYARTSMTKNLMRIYHDSMENALSCWLT
EHNCPYSDQISYLPPKQRABWGPNWSNRMCIRVCRLDRVSTSLRGRALSAFED
RAAARALHLAIVAFASQWTQHAQRGAGLSVPADIAGDERAIRRNAWNEARHA
LQHTTGIPSFRVIFANIIFSLTQSVLDDTEQQNVGARLDRLLENDGAPVFLETANR
QLYTFRHKFARMQRRGKAFNRLPVESVASTFADTFETPTPPSESPQLDPVVASEE
HRSTLSLMFWLGIMFDTLSAAMYQRPLVVSDEDSQISSAYPSTRGSETPINLDC
WEPPRQAPSNQEKSDVWGDLFLRTSDSLQGHESHTQISQPAARWPCTYEQAAA
ALSSATPVKVLLYRRVTQLQTLLYRGASPARLEAAIQRTLHYYNHWTAKYQPF
MQDCVANHELLPSRIQSWYVILDGHWHLAAMLLADVLESIDRDAYSDINHIDLV
TKLRLDNALAVSALARSSLRGQELDPGKASPMYRHFHDSLTEVAFLVEPWTVV
LIHSFAKAAYILLDCLDLDGQGNALAGYLQLRQNCNYCVRALQFLGRKSDMAA
LVAKDLERGLNGKVDSFL
SEQ ID NO 129 Amino acid sequence of A. cleistominutus AlcR protein
CDPCRKGKRRCDAPENRNEANENSWVSCSNCKRWNKDCTFNWLSSQRSKPKG
AAPRARTKKARAATTTSEPSTSAAAFPTPESDNHDAPPVINAHDALPSWTQGLL
SHPSDLFDFSQSSIPANVEDAAANVQSDAPFPWDLAIIGDFSMGQQLEKPLSPLS
FQAVLLPPHSPNTDDLIRBLEEQTTDPDSVTDTNSLQQAAQHGSLWSDRHSPLLP
ENSLCMASDSTARRYARSSMTKNLMRIYHDSMENALSCWLTEHNCPYSDQISY
LPPKQRAEWGPNWSNRMCIRVCRLDRVSTSLRGRALSAEEDRAAARALHLAIV
AFASQWTQHAQRGAELSVPADIAADERAIRRNAWNEARHALQHTTGIPSFRVIF
ANIIFSLTQSVLDDTEQQGVGARLDRLLENDGAPVFLETANRQLYTFRHKFARM
QRRGKAFNRLPGGSVASTFADIFETPTPSSESPQLDPVVASEEHRSTLSLMFWLGI
MFDTLSAAMYQRFLVVSDEDSQISSASPSTRGSETPLNLDCWEPPRQVPSNQDKS
DVWGDLFRASDSLQDHESHTQISQPAARWPCBQAAASATPVKVLLYR
RVTQLQTLLYRGASPARELEAAIQRTLHVYNHWTAKYQPFMQDCVTNHELLPSR
IQSWYVILDGHWHLAAMLLADVLESIDRDSYSDINHIDLVTKLRLDNALAVSAL
ARSSLRGQELDPGKASPMYRHFHDSLTEVAFLVEPWTVVLIHSFAKAAYILLDC
LNLDSQGNALAGYLQLRQNCHCCIRALQFLGRKSDMRLLR
SEQ ID NO 130 Amino acid sequence of A navahoensis AlcR protein:
CDPCRKGKRRCDAPENRETNBNGWASCSNCKRWNKDCTFNWLSSQRSKPKG
AAPRARMKKARTAAATAEPSNSATAMPTPBSGHQDTPPIINAYDALPSWSQGL
VSHPGDLFDFSQSSIPMHTDDAVNVQSEVPFPWDLAPGDFSSMGQQLENPLSPL
SFQAVILPPHSPNTDDLIHELEEQSTDSTKFAGLRRDTPDGSLWSSRASPLAPQNS
LCIASDKTAQQYARSSMTKNLMIUYHDSMBNALSCWLTEHNCPYSDQTSYLPP
KQRAEWGPNWSNRMCIRVCRLDRVSTSLRGRALSAEEDRAAVRALNLAIVAFA
SQWTQHAQKGAGLSIPTDIAGDERAIRRNTWNEARHALQRSTGIPSFRVIFANIIF
SLTQSVLDDSEQQGAGTRLDKLIENDRAPLFLETANRQLCTFHKFARMQRRR
STADQLRRVSAASALADWETPTPSPGSPHLDPILANEEHRSTLSLMFWLGIMFDT
LSAAMYQRPLVVSDEDSQISSASPSTQOSETPINLDCWEPPRQIPNDRAKSDVW
GDLFLRDSDSPQHDKSRAQISQPAARWPCTYEQAAAVLSSATPVKVLLYRRVT
QLQTLLYRGASPARIEAAIQKTIHVYQHWTEKYQPFMQDCVANHELLPSRIQS
WYVILDGHWHLAAMLLADVLESIDRDTYSDIDHTDLVTKLRLDNALAVSALAR
SSLRDQEQCPDKASQMYRHFHDSLTEVAFLVEPWTVVLIHSFAKAAYILLDCLD
VDGQRSTLAGYLQLQQNCNYCIRALQYLGRKSDMRLLR
SEQ ID NO 131 Amino acid sequence of A. heterothallicus AlcR protein
CDPCRKGKRGCDAFELVGVQTFLTMIQEIRSGDGYTCSNCKRWKKKCTFNIWS
SRRADARSVAANSRAKAKPTSTPVVATTASVATSVVAPPTPDSGNFPAMLNMGI
NTSEYNALLDEGLRSSQLDPARFGDMFEFMSPSNFAAEVLHAQSAIGGVNETLA
WTMGVPGSWPMGMMPQSFSLSSLQSQELFISNEDANPYDVIQQLEDDFEDPA
TSVSKRDEDVRKFQWELCIASDKTANKVGRSTMNGNLIRIYHDSMENALSCWL
TEHNCPYADPMSAMLPFNQRKEWGPSWSNRMCIRVCRLDRASSSIRGRALSVE
EDRTAARALHLAIVAFASQWTQHAQKGTGLSVPAGIAYDERSTRKNIWNEARH
ALQHSTGIPSFRVVFANFSLTQSPLDETRPAKLAQLLDNDGAPVFLENANRQLY
TFRHKFARLQREAPPPAATDLRRGSISSTLTEVLEIPTPESPQLDPILASQDHRSTL
SLLFWLGIMFDTLSSAMYQRPLVVSDEDSQIGSASPTASADHRVNLNYWEIPDN
DLPAKNDVWGEFFLQPAARQEPTSTHPQLQPQQPRWPCSYEEAASVLSEATPV
KVLLYRRTTQLQTLIYRGSSPARLEEVIQKTLLVYHIHWTCTYQSFMLDCVANHE
SLPHRIQSWYVILDGHWHLAAMLLADVLESIDRSYLGMESERBSRIASDLIATLRI
DNALAVGALARASLHGQNSTMIIRYFHDSLNEVAFLVEPWTVVLIHSFAKAAYI
SLDCLGQGQGGALAECFRQNCEYCICALKYLGRKSDMRL
SEQ ID NO 132 Amino acid sequence of A. spectabilis AlcR protein:
CDPCRKGKRGCDAPENRTEILFSSCSNCKKWKKEGTFNWLSTNPTLKAKGNQE
KKRRKTKAKPCTVAADTSTDTATPDDSVGIPSIGSDVGISVGDGSYGGFIDDGLQ
SAQWFPVNPGDGDVFALPGTGLLDLPSSSLLFSEAGIGGNDTSDPYAQSLVSWNI
GFPDSSQLDAVPGKSFTRLDSLPTDSLDYRFDVIQQLEEELAQDSRTFPSGFCMA
SDNTAKAYARSTMTHNLLRIYNDGMENALSCWLTEHNCPYTDSIGDLLLPYSQ
RKEWGPDWSNRMCIRVCHLDRASSLIRGRALSAEEDKTAARALHLAIVAFASQ
WTQHAQRGPVLSVPAGIDEDERLIKKDVWNEARHALEHSTRIPSFRVIFANIIFSL
TQSPLDKGDRRDQGLGQLLENDSAPIFLRNANRQLYTFRHKFTKLQRSNRNSPQ
VDPILSSQDIIRSTLNLLFWLGIMFDTLSAAMYQRPLVVSDEDSQTTSISPPPTPAP
LNSPAQINLDCWDLPSDQPQTTTLTLRQKQDVWGDFFLHPSPSLSHQEPTTQLN
PHPQLEHPKRWPCTYAEPASILSSATPVKVLLYRRVTQLQNUYRGATPSQLELV
IQKTLLVYNHWQQTYAPFMTDCVTNHAILPPRIQSWYVILDGHWHLAAMLLAE
VVEEIDNARLGLDSARETRNISNFVETLRRENALAVGALARASLQGQNPGMEER
YHDSVNBVAFLVEPWTVVLVNCFAKGGYISAERAAGCSSFTGAGVGAGDGIGV
GEVFRLNCGFCICALEYLGRKSDMRLL
SEQ ID NO 133 Amino acid sequence of A. bicolor AlcR protein:
CDPCRKGKRGCDXXBNRTEILFNSCSNCKKWKKECAFNWLATNPTTKGKGNQE
KNRRTKAKPSTAATDTNTAIATPDDSVDIPSVGSDVGISVGDGSYGSCIDDGLQS
AQWFPVNPGNGDVLALPGTGLFDLTSSSLLFPEGGIGGNDTSDPYAQSIISWNM
GGFPDNWQLGAVPGKSFARLDIYTNSLDDTFDIIQPLEEDSSRNSRtFPSGFCIAS
DNTAKAYARSTMTRNLLRIYHGSMDNALSCWLTEHNCPYIDSIGDLLLLYSQRK
EWGPNWSNRMCIVCQLDRASSSIRSRALSAEEDMTMVFASQWTQHAQRGPVL
SVPAGIDENBRSLRKNYWDEIRHAQEHSTRIPSFRVIYAFANTTFSLTQSPLDKGER
RGdGLGQLLENYSAPIFLENTNRQRYPFRHKFTRLQRRNRSSPQVDPILSSQDHR
GTLNLLFWFGIMFDTLSAAMYQRPLVVSDEDSQIASISPPPPTPSPLNPPAQNNLE
CWNFPSDQPQTTTLTIRqKQDVWGYSFLHPTASLSHQEP2TTQLNPHPQPKHRPK
RWPCTYAESASLLSFATPVKVLLYRRVTQLQTUYRGAAPSQLESVIQKFLLVYN
HWQQFYAPFMTDYVTNHAILPPRIHSWCVMLDGHWHLAAMLLAVVVEETDN
AGLGLDSAREARNLSDFVGILRRENALAVGALARAPLQGQNPGMEEHYHNSL
NEVAFPVEPWAAVLVYCFAKGGGGLYIPLERVGYSSFIRDGSGDGVKDGKVFR
LNCELCICVSEYLGRKSDMRLGG
SEQ ID NO 134 Alcvers seq4r: CAAATTQTGCGTCATCGTTG
SEQ ID NO 135 Alcvers seq 5r: GGAAGCGAACATATCATTG
SEQ ID NO 136 Alcvers for: GQTTGCTCGCCATGGATGAC
SEQ ID NO 137 Alcust for: CTCGAATGAAGATGGGAGACTC
SEQ ID NO 138 Alcust rev: TTACACAAGGATATCCGCTGAC
SEQ ID NO 139 Alcflav seq6r: GAAGATCGAAAGTGTGATG
SEQ ID NO 140 Alcflav for: ATGTCTTATCGTCGCCGTCAG
SEQ ID NO 141 Alcflav seq 7r: ACTCTCCACACTCGTGAG
SEQ ID NO 142 Alcflav seq 8r: CCATTGAGAGTCATGTCG
SEQ ID NO 143 Alcfum for: ATGGAGGCTCATCGTCGACGCCAG
SEQ ID NO 144 Alcfum RT: CAAAGCCAGGTGGCGAAGAG
SEQ ID NO 145 ITS: TCC GTA GGT GAA CCT GCG G
SEQ ID NO 146 ITS: TCC TCC GCT TAT TGA TAT G
SEQ ID NO 147, The sequence of the alcA promoter region from Aspergillus nidulans:
TAAGTCCCTTCGTATTTCTCCGCCTGTGTGGAGCTACCATCCAATAACCCCC
AGCTGAAAAAGCTGATTGTCGATAGTTGTGATAGTTCCCACPTGTCCGTCCG
CATCGGCATCCGCAGCTCCGGATAGTTCCGACGTAGGATTGGATGCATGCG
GAACCGCACGAGGGCGGGGCGGAAATTGACACACCACTCCTCTCCACGCAG
CCGTTCAAGAGGTACGCGTATAGAGCCGTATAGAGCAGAGACGGAGCACTT
TCTGGTACTGTCCGCACGGGATGTCCGCACGGAGAGCCACAAACGAGCGGG
GCCCCGTACGTGCTCTCCTACCCCAGGATCGCATCCTCGCATAGCTGAACAT
CTATATAAAGACCCCCAAGGTTCTCAGTCTCACCAACATCATCAACCAACA
ATCAACAGT

1. A polypeptide capable of activating an alc inducible promoter in the presence of a chemical inducer, provided that the polypeptide does not have the amino acid sequence specified in SEQ ID No 121.

2. A polypeptide according to claim 1 comprising at least a first motif having an amino acid sequence selected from the group consisting of SEQ ID Nos. 104, 105, 106, 107, 108, 109, 110, 111, 112, 113, 114, 115, 116, 117, 118, 119, and 120.

3. A polypeptide according to claim 2 at least a second motif having an amino acid selected from the group consisting of: SEQ ID Nos 0.104, 105, 106, 107, 108, 109, 110, 111, 112, 113, 114, 115, 116, 117, 118, 119, and 120; wherein the second motif is not the same as the first motif.

4. A polypeptide according to any one of the preceding claims, which comprises the consensus amino acid sequence specified in SEQ ID No 123.

5. A polypeptide according to any one of the preceding claims, which has an amino acid sequence selected from the group consisting of: SEQ ED Nos. 124, 125, 126, 127, 128, 129, 130, 131, 132, 133, 59, 18, 62, 66.

6. A polypeptide according to any one of the preceding claims, the amino acid sequence of which comprises a plurality of at least doublet repeats of amino acid residues, wherein the plurality of at least doublet repeats comprise greater than 7.5% of the polypeptide.

7. A polypeptide according to any one of claims 1 to 6, the amino acid sequence of which comprises a plurality of at least triplet repeats of amino acid residues, wherein the plurality of at least triplet repeats comprise greater than 1% of the polypeptide.

8. A polypeptide according to claim 6, which comprises at least 6 doublet repeats of leucine, at least 3 doublet repeats of serine and at least 4 doublet repeats of threonine.

9. A polypeptide according to claim 6, which comprises at least 3 doublet repeats of alanine, at least 1 doublet repeat of cysteine, at least 1 doublet repeat of aspartic acid, and at least 1 doublet repeat of proline.

10. A polypeptide according to claim 8 or claim 9, which comprises a plurality of at least triplet repeats of amino acid residues, wherein the plurality of at least triplet repeats comprise greater than 1% of the polypeptide.

11. A nucleic acid encoding a polypeptide according to any one of the preceding claims.

12. A nucleic acid according to claim 11, comprising a sequence selected from the group consisting of: SEQ ID Nos. 102, 101, 103, 76, 74, 75, 78, 77, 38, 61, 46, 65, 79, 73, 58, 17.

13. An expression cassette comprising
(i) a first promoter,
(ii) a first nucleic acid according to claim 11 or claim 12, wherein the polypeptide encoded thereby is capable of activating an alc inducible promoter in the presence of an exogenous chemical inducer, the nucleic acid being under the control of the first promoter,
(iii) a second promoter that is inducible by the polypeptide encoded by the first nucleic acid in the presence of the exogenous chemical inducer, and
(iv) a second nucleic acid, the expression of which is under the control of the second promoter.

14. An expression cassette according to claim 13, which is a plant gene expression cassette.

15. An expression cassette according to claim 13 or claim 14 wherein the first promoter is a constitutive promoter.

16. An expression cassette according to any one of claims 13 to 15, wherein the second promoter is an alcA, aldA, alcB, alcR or alcC promoter of a fungal species, or is a chimeric promoter containing a regulatory sequence derived therefrom.

17. An expression cassette according to claim 16 wherein the second promoter is an alcA promoter from an Aspergillus species.

18. An expression cassette according to claim 17 wherein the second promoter is an alcA promoter of Aspergillus nidulans.

19. An expression cassette according to claim 18 wherein the second promoter comprises SEQ ID NO 147.

20. An expression cassette according to claim 16 wherein the second promoter is an alcR promoter from an Aspergillus species.

21. An expression cassette according to claim 20 wherein the second promoter is an alcR promoter from Aspergillus nidulans, Aspergillus ustus, Aspergillus flavus, or Aspergillus versicolor.

22. An expression cassette according to claim 21 wherein the second promoter comprises SEQ ID NO 39, SEQ ID NO 47, or SEQ ID NO 60.

23. A cell comprising an expression cassette according to any one of claims 13 to 22.

24. A cell according to claim 23, which is a plant cell.

25. A cell according to claim 23 or claim 24, wherein the expression cassette is stably incorporated into the genome.

26. A plant, or progeny or seeds thereof, comprising cells according to claim 23 or claim 24.

27. A method for controlling gene expression in a cell, comprising transforming a cell with a expression cassette according to any one of claims 13 to 19, and applying an exogenous chemical inducer to the cell in order to induce transcription of the second nucleic acid.

28. A method according to claim 27 wherein the exogenous chemical inducer is an alcohol, ketone, or ester.

29. A method for controlling gene expression in a plant according to claim 26, comprising applying to the plant an exogenous chemical inducer to induce transcription of the second nucleic acid.

30. A method according to claim 29 wherein the chemical inducer is butan-2-one (ethyl methyl ketone), cylcohexanone, acetone, butan-2-ol, 3-oxobutyric acid, propan-2-ol, ethanol or a compound of formula (I) 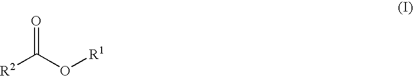
in which R¹ is a lower alkyl, lower alkenyl or lower alkynyl group, and R² is an organic group such that R²COOH is an agriculturally acceptable acid.

31. An alcR promoter sequence obtainable from Aspergillus ustus, Aspergillus flavus, Aspergillus versicolor or Aspergillus fumigatus, which acts as an inducible promoter in the presence of an AlcR regulator protein and an exogenous chemical inducer.

32. An alcR promoter sequence according to claim 31 which comprises SEQ ID NO 39 (ustus), SEQ ID NO 47 (versicolor), SEQ ID NO 60 (flavus) or SEQ BD NO ?? (nidulans).

33. A chimeric alcR promoter which acts as an inducible promoter in the presence of an AlcR regulator protein and an exogenous chemical inducer, the promoter comprising at least part of an alcR promoter sequence according to claim 31 or claim 32 and a heterologous promoter region.
